(19) United States
(12) Patent Application Publication
(10) Pub. No.: US 2005/0153357 A1
(43) Pub. Date: Jul. 14, 2005

(54) Bioactive sensors

(76) Inventors: Jutta Eichler, Dossenheim (DE); Michael Frank, Heidelberg (DE); Holger Ottleben, Heidelberg (DE); Harald Rau, Dossenheim (DE); Renate Sekul, Ladenburg (DE); Dirk Vetter, Heidelberg (DE)

Correspondence Address: KAGAN BINDER, PLLC, SUITE 200, MAPLE ISLAND BUILDING, 221 MAIN STREET NORTH, STILLWATER, MN, 55082, US

(21) Appl. No.: 11/077,907

(22) Filed: Mar. 10, 2005

Related U.S. Application Data
(63) Continuation of application No. 09/995,491, filed on Nov. 27, 2001.

(30) Foreign Application Priority Data
May 26, 2000 (WO) PCT/DE00/01705
May 28, 1999 (DE) DE 199 24 606.8

(51) Int. Cl. C12Q 1/68; G01N 33/53; C12M 1/34

(52) U.S. Cl. 435/6; 435/7.1; 435/287.2

(57) ABSTRACT

The present invention relates to sensors for the detection of molecular interactions between immobilized ligands and non-immobilized interaction partners (receptors). These surfaces use novel ligand-anchor conjugates which allow highly specific interaction with suitable interaction partners. Furthermore the invention relates to methods of providing the sensing surface and in particular methods of synthesising the ligand-anchor conjugates (LAC).

### PRIORITY CLAIM

The present Continuation patent application claims the benefit of U.S. National Stage patent application Ser. No. 09/995,491, filed Nov. 27, 2001, entitled BIOACTIVE SENSORS, which claims the benefit of International Application No. PCT/DE00/01705, filed on May 26, 2000, and having a PCT Publication No. WO 00/73796, entitled BIOACTIVE SENSORS, and also claims priority from DE 199 24 606.8, which was filed on May 28, 1999, wherein all prior patent applications are commonly owned by the owner of the present patent application and wherein the entireties for all purposes of said applications and prior patent applications are incorporated herein by reference.

### FIELD OF THE INVENTION

The present invention relates to sensors for detecting molecular interactions between immobilized ligands and non-immobilized interaction partners (receptors). The surfaces of these sensors exhibit novel ligand-anchor conjugates (LACs) which allow highly specific interaction with suitable interaction partners. Moreover, the invention relates to methods for providing the sensor surface and in particular for synthesising the ligand anchor conjugates.

### BRIEF DESCRIPTION OF THE DRAWINGS

FIG. 1 is a schematic view of a design of an anchor molecule according to the invention.

FIG. 2 is a schematic view of another design of an anchor molecule according to the invention.

FIG. 3 is a schematic view of another design of an anchor molecule according to the invention.

FIG. 4 is a schematic view of an LAC structure according to the invention.

FIG. 5 is a synthesis scheme for synthesis of LAC based on anchor 1.

FIG. 6 is a synthesis scheme for synthesis of LAC based on anchor 2.

FIG. 7 is a synthesis scheme for synthesis of LAC based on anchor 3.

FIG. 8 is a synthesis scheme for the combinatorial synthesis on anchor 1.

FIG. 9 is another synthesis scheme for the combinatorial synthesis on anchor 1.

FIG. 10 is a synthesis scheme for synthesis of LAC based on anchor 7.

FIG. 11 is a structural representation of anchor 8.

FIG. 12 is a schematic presentation of the synthesis of Fmoc-Std-OH.

FIG. 13 is a structural representation of anchor 9.

FIG. 14 is a synthesis scheme for synthesis of LAC based on anchor 11.

FIG. 15 is a scheme for the synthesis of anchor 13.

FIG. 16 shows a synthesis scheme of the present invention.

FIG. 17 is a synthesis scheme for synthesis of LAC based on anchor 15.

FIG. 18 is a structural representation of LAC 1.

FIG. 19 is a structural representation of an anchor molecule which has an acetyl group (non-ligand) at its amino N atom.

FIG. 20a is a structural representation of anchor 1.

FIG. 20b is a structural representation of anchor 2.

FIG. 20c is a structural representation of anchor 3.

FIG. 20d is a structural representation of anchor 4.

FIG. 20e is a structural representation of anchor 5.

FIG. 20f is a structural representation of anchor 6.

FIG. 20g is a structural representation of anchor 7.

FIG. 20h is a structural representation of anchor 8.

FIG. 20i is a structural representation of anchor 9-1,9-2, 9-3, and 9-4.

FIG. 20j is a structural representation of anchor 10.

FIG. 20k is a structural representation of anchor 11.

FIG. 20l is a structural representation of anchor 12.

FIG. 20m is a structural representation of anchor 13.

FIG. 20n is a structural representation of anchor 14.

FIG. 20o is a structural representation of anchor 15.

FIG. 20p is a structural representation of anchor 16.

FIG. 20q is a structural representation of anchor 17.

FIG. 20r is a structural representation of anchor 18.

FIG. 21 is a schematic cross-sectional view of a carrier plate of the present invention.

FIG. 22 is a schematic cross-sectional view of another carrier plate of the present invention.

FIG. 23 is a schematic cross-sectional view of another carrier plate of the present invention.

FIG. 24 is a CCD picture of four fields of a gold-coated carrier plate as a sensor surface.

FIG. 25 is a CCD picture of fields during a luminescence reaction.

FIG. 26 illustrates a parallel luminescence measurement by means of a CCD camera at varying concentrations after a plate had been treated.

### DESCRIPTION

The modification of organic or inorganic surfaces is not only used for purifying biomolecules (such as the adsorption of nucleic acids on carriers as disclosed by Qiagen, Hilden, Germany in WO 95/01359 or the cross-linkage of a dextran polymer matrix for affinity chromatography or gel filtration, Sephadex® of Pharmacia, Uppsala, Sweden) but also for biomolecular interaction analysis.

Biomolecular interactions are studied by means of known methods of interaction analysis in receptor-ligand systems, wherein the receptor is usually a biomacromolecule (such as a protein or a single-strand DNA) and the ligand a “probe,” a predominantly low molecular weight molecule of biological or synthetic origin (peptides, oligonucleotides or so-called small organic molecules). Such ligands exhibit very specific structural features which may interact with the receptor if the latter possesses corresponding structural units. Bonds with the receptor may be developed by one or more ligands. In the pharmaceutical and agrichemical industries, interaction analysis is used for drug discovery programs. In particular in such programs, a maximum number of different samples is to be analyzed in a minimum time period (high-throughput screening, HTS). Moreover, interaction analysis is used for studying genomes (polymorphism (SNP) or expression pattern analysis) or for food analysis.

It is useful in practice to covalently bind or adsorb one of the potentially binding partners, receptor or ligand, to an organic or inorganic surface. By generating a specific boundary layer on the surface (immobilizing ligand or receptor), such a boundary layer is provided with bioactivity. Immobilizing a binding partner facilitates processing, such as the implementation of washing steps, and, in combination with a suitable, most often optical detection method (such as fluorescence dtion), indicates the presence and extent of interactions between receptor and ligand on a molecular level.

Bioactive surfaces can usually be generated in several steps. Films of organic monolayers (Bain and Whitesides, Angew. Chem. 101 (1989) 522-8; Zhong and Porter, Anal. Chem. (1995) 709A-715A) have particular advantages (physicochemical stability, structural unity). In a first step, thiols are chemisorbed on gold. Thus, long-chain alkyl thiols are packed in the form of a self-assembled monolayer (SAM) onto the solid phase, the gold atoms being complexed by the sulphur functional groups. Such SAMs are known from the literature and have been characterized by means of various physical methods. Poirier and Pylant, Science, 272 (1996) 1145-8 disclose scanning tunnel microscopic images of such monolayers on gold.

If the SAM-alkane chain ends are, for example, provided with a hydroxy group (“omega-functionalized”), so-called hydrophilic spacer moieties (such as dextran) may be attached in subsequent reactions. Here, the dextran acts as a protein adsorption-resistant hydrogel and reduces the unspecific binding (passive adsorption) of the biomolecules to be analyzed. The modification (carboxymethylation) or oxidation of the dextran leads to randomly distributed carboxyl groups which are suitable for bioconjugation reactions. The carboxylates are subsequently chemically activated by formation of so-called active esters. In a second step, which is also called “conjugation step”, these active esters are covalently bound to a ligand or receptor containing a primary amino group (Biacore® method). The latter step yields a synthetic, bioactive surface. Surfaces on which the activation and conjugation steps are not implemented or on which conjugates are formed by binding so-called “non-ligands” which cannot be expected to show any bioactivity usually serve as so-called negative controls in interaction studies. Usually, very small organic groups, such as acetyl, methyl or aminoethyl groups, are applied as non-ligands.

Other methods for generating bioactive surfaces use the following molecular layer structures:
silanization of glass or silicon with reactive epoxide- or amino group-containing silanes, subsequent acylation of the amino groups, for example with nucleoside derivatives (Maskos and Southern, Nucl. Acids Res. 20 (1992) 1679-84);
passive adsorption of polylysine on glass, subsequent DNA deposition by non-covalent electrostatic bonds (Schena et al., Science, 270 (1995) 467-70);
passive adsorption of protein on polystyrene (conventional ELISA technique);
passive adsorption of vesicles or micelles on SAMs or hydrophobic silane layers (Sackmann, Science, 271 (1996) 43-47);
passive adsorption of spread-out lipids on glass (Langmuir-Blodgett technique);
passive adsorption of proteins or peptides on cellulose nitrate or other membrane materials (conventional “dot blot” technique).

On the basis of these methods, relatively complex multilayer systems can be realized. An amino polysiloxane, for example, can be biotinylated on glass or an SAM on gold. Avidin may be applied thereon which is capable of binding biotinylated ligands or receptors (Müller et al., Science, 262 (1993) 1706-8). A further example consists in the so-called “His-tag” or nickel-NTA surface which binds ligands or receptors carrying a histidine oligomer motif by metal complexing.

Any of the aforementioned methods entails the tremendous disadvantage that the bioactive component (receptor or ligand) is introduced in a final, late step in a multi-stage surface modification process. Thus, a complex structure is formed on the surface which is hard to characterize and whose properties, such as coverage density and constitution, are difficult to determine. An important issue in the design of bioactive surfaces is to obtain detailed information on the molecular constituents of the surfaces. The detected activities can be correlated with the responsible chemical or biological structures only with difficulty without this information. A detailed chemical analysis of surfaces modified in multi-step methods is, however, quite difficult on account of the naturally very small amounts of substances available. So far, it could only be shown that the presence of receptors non-covalently attached to bioactive surfaces is detectable by means of laser-desorption mass spectrometry (Nelson et al., Anal. Chem., 69 (1997) 4363-8). For comparing binding phenomena on bioactive boundary layers, exact knowledge of the chemical substances constituting these layers is indispensable since it is known that even small structural differences may have drastic effects on the molecular interaction. The techniques which are presently available for a direct physicochemical characterization of monolayers (such as XPS, FT-IR) are not capable of contributing to a detailed structural analysis. The high stability of the SAM or silane films preclude non-destructive desorption and analysis with high-resolution methods, such as MS.

The presently best characterized bioactive layers are obtained as described above by contacting dissolved alkyl thiols with a gold surface. The self-assembled monolayers (SAMs) thus obtained have been characterized in detail by numerous physical detection methods, and the structural properties of these surfaces are well-known. However, this advantage is counterbalanced by the deposition of macromolecular layers on an SAM as used in the Biacore® method. New chemical bonds to a heterogeneous dextran matrix with a non-uniform structure are practically established by “trial and error” and may only be examined by indirect detection methods. This is not only unfavourable for the optimization of the reaction parameters but also considerably disadvantageous in view of the immobilization of a multitude of samples. A “control” of the surface structures on a molecular level is no longer given.

In the aforementioned Biacore® system (Biacore AB, Uppsala, Sweden), the bioactive surface is used in form of a sensor chip with a thin gold surface and a monolayer of organic molecules which is immobilized thereon and which, in turn, is bound to an organic matrix, in particular a dextran matrix. Such a structure is described in WO 90/05303. Usually, the sensor chip is inserted into the sensing device and subsequently “activated”, i.e. chemically reactive groups which enable a further functionalization of the surface by means of ligands are inserted into the matrix. The ligands to be immobilized are subsequently contacted with the surface, which leads to their covalent binding to the matrix. Then excessive reactive groups are saturated with another, preferably low-molecular substance which is not capable of interacting with the test substances. Only then is the sensor chip in principle prepared for detecting substances interacting with the ligand immobilized on the surface. The actual measurements are carried out by means of surface plasmon resonance (SPR).

This method, however, is disadvantageous in that the sensor surface, i.e. the binding matrix, has first to be activated or prepared as explained above in one or more steps which are carried out in a flow system within the sensing device. Furthermore, the branched organic matrix forms a gel-like layer on top of the gold surface and, after preparation of the binding matrix and immobilisation of the ligands, contains the ligands randomly distributed not only on the surface, but also within the matrix. The reaction conditions cannot be controlled during the preparation of the sensor surface in such a way that an exactly defined surface structure is formed. Thus, diffusion effects of the analyte into the strongly hydrated organic matrix frequently become relevant so that diffusion limitation of the interaction between analyte and immobilized ligand may occur. In such cases, reliable statements on the kinetic or thermodynamic parameters of the interaction can no longer be made. Schuck and Minton have already addressed this problem which is due to an undefined surface (Schuck & Minton, Trends Biochem. Sci. (1996) 21 (12): 458-460).

In particular during the immobilisation of lipids on a modified gold surface, which is, for example, frequently carried out by use of micellar solutions or lipid vesicles containing membrane proteins, the additional coverage of the chip surface (or the binding matrix) and the layer thickness of the yielded lipid layer is no longer exactly defined. This is due to the fact that the process of membrane formation and fusion and synthesis, e.g. spreading out a monolayer by using a film balance, cannot be accurately controlled since many measurements, such as an exact determination of the surface coverage density and the layer thickness, cannot be applied any more to a sensor chip exhibiting an immobilized dextran or lipid layer and the biomolecules optionally bound thereon. Since SPR-based methods are as a rule used in cycles, i.e. several series of measurements in succession are carried out on the same sensor surface (or the same sensor chip), there are also accumulation and abrasion effects at the surface which additionally obstruct measurement and evaluation.

EP-A-0 574 000 describes a method of producing a binding matrix comprising a carrier material, such as gold or silver, and an “affinity carrier” bound thereon, such as biotin, which is capable of binding to at least one free reactant, such as streptavidin or avidin. This affinity carrier forms an essentially laterally homogeneous binding layer which is diluted by non-interacting groups on the surface of the carrier material. The carrier material is incubated with an aqueous reaction solution comprising the affinity carrier linked to the layer forming part of the molecule via a short-chain spacer molecule and at least one hydrophilic diluting molecule so that a so-called “mixed” self-assembling monolayer is formed on the carrier material.

However, the affinity carrier does not act as a ligand or receptor in this binding matrix but merely serves for forming further layers by non-covalent bonds to avidin or streptavidin, which, in turn, is capable of binding biotinylated ligands or receptors.

This method entails the further disadvantage that relatively large volumes of the conjugates of the affinity carrier and the anchor compounds, which serve for binding to the surface, are synthesized in a homogeneous solution. Comparatively large amounts of the substance have to be used and complicated steps, such as column chromatography or extraction, become necessary. Thus, the use of this method for producing a multitude of different ligands, for example in new drug screening methods (HTS), is particularly problematic. In such screening methods, miniaturizable methods with a high sample throughput are of particular interest as they allow for a parallel measurement of interactions between a multitude of different ligands with one or more biomolecules of interest. Frequently, chemical modifications of a basic ligand structure are necessary or desirable, which can be produced e.g. via methods of combinatorial chemistry, in order to measure the influence of such modifications on affinity or specificity with high efficiency, for example in view of bonding, inhibition or activation of an enzyme. For this application, however, the aforementioned method is unsuitable.

A further example for a known binding matrix, which is also termed binding film, is disclosed in WO 92/10757 which also describes an affinity carrier adsorbed on a carrier material by anchor groups.

WO 98/31839 describes the immobilisation of nucleic acids on surfaces suitable for electron transfer measurements, a complexing agent being used.

The mode of function of “Biacore® ” has already been discussed above. It uses the SPR measuring principle, which has hardly been used until recently and detects changes in the layer thickness at surfaces and is therefore mass-sensitive. SPR allows for real-time observation of the biomolecular association without any chemical, radiochemical or immunochemical labelling and with a very low substance consumption.

In this method, the light reflected from a thin gold layer is detected. At a suitable resonance condition (angle of incidence and wavelength of the light and thickness of the gold layer), the intensity of the reflected light decreases. The light energy is then transformed into charge density waves of the electron gas in the gold layer. These charge density waves are called plasmons. For observing resonance, either monochromatic light is used and the intensity of the reflected light as a function of the angle of incidence is recorded or the angle of incidence is kept constant and the wavelength of the light is varied. The resonance condition may be varied by coating the side of the gold layer facing away from the incident light. The receptor or ligand is immobilized on the gold surface. Upon addition of the ligand or receptor, the resonance condition is changed if these molecules attach.

In 1989, Pharmacia (Uppsala, Sweden) launched the first biosensor based on SPR measurements.

The SPR method is advantageous because of its high accuracy when determining the refractive index and layer thickness of thin dielectric layers. The application of SPR spectroscopy in biochemical analysis has therefore increased in the past years since it allows for direct examination of biomolecular interactions. For this purpose, a reactant (ligand) is immobilized on the carboxydextran SAM gold surface and the other reactant (analyte, receptor) is dissolved and contacted with the sensor surface, e.g. in a flow system. The interaction is directly detectable as an increase in the layer thickness.

The SPR measuring method has turned out to be very effective in various fields and is considered an established technique. Therefore, it should be possible to explore new areas of application for SPR sensors, such as high-throughput screening (HTS).

The following methods number among alternative biosensor methods which do not require labelling of the target molecule with fluorescent dyes, groups showing high affinity (biotin) or radioactive elements which are careful and economical regarding the often very precious biomacromolecules:
quartz micro balances and
reflectometric interference spectroscopy (RIFS).

In biosensors based on quartz micro balances, the bonds between receptors and ligands are measured by means of the weight increase affecting the frequency of oscillation of the quartz crystal (Ebara and Okahata, JACS 116 (1994) 11209-12). This sensing method is still being developed and the respective sensing devices are not commercially available. Their use for bioanalytical purposes is hardly documented.

Reflectometric interference microscopy is capable of using the partial reflection of light at interfaces for detecting changes in the layer thickness. The attachment of biomolecules to binding partners (ligands) causes a shift in the intensity profile as a function of the wavelength. The shift of the detected curves is proportional to the change in the layer thickness. However, gold/SAM surfaces cannot be used in RIFS.

It is the object of the present invention to provide sensors on the basis of exactly defined SAM forming molecule structures which are in particular applicable in HTS. In order to avoid the aforementioned disadvantages of the prior art, the structural motifs (ligands) relevant for the bioactivity are to be combined in preceding steps with an SAM forming anchor and can then be completely analytically characterized. Only after a complete synthesis are these conjugates of ligands and anchors (ligand-anchor conjugates, LACs) immobilized on a suitable surface, thus forming a biospecific boundary layer in form of a monolayer of bioactive LACs. Methods of solid-phase synthesis known in the art have turned out to be advantageous for the LAC synthesis. In such methods, the target structure is prepared starting from a solid surface. Thus, first the anchor and then the ligand bound thereto can be synthesized in several individual steps. Optionally, a presynthesized ligand may also be bound in a single step to the anchor. Such a synthesis method allows for the provision of ligand-anchor conjugates whose structure is optimized for use in screening methods in form of SAMs. The advantages of combining the principles of combinatorial or highly parallel synthesis and of SAM formation have so far not been disclosed in the prior art.

In the present invention, ligands mean structural elements which may specifically interact with test substances or their subunits on account of their structural features. By means of ligands, receptors with compatible structural units may be immobilized on a sensor surface for example during screening tests. With the ligand structure being known, conclusions on the structure of the receptors may thus be drawn.

The terms “ligand” and “receptor” are often not consistently used in the literature. Therefore, it should be noted that the present invention uses the term “ligand” for molecules whose terminals are, preferably covalently, bound to the anchor. For interaction analysis, ligands are immobilized by means of such anchors on the respective sensor surfaces, thus providing a biospecific boundary. Examples of such ligands are peptides, oligonucleotides or small organic molecules.

“Receptors” are molecules, preferably biomolecules, which are present in the medium to be analyzed. The interaction analysis is based on their capability of interacting with the aforementioned boundary layer or the ligands thereon.

The receptor molecules are preferably bound by the ligands in the course of the measurement on account of specific, corresponding steric or electronic structures in both molecules. Ionic or polar, van der Waals' or other hydrophobic interactions or hydrogen bonds are to be considered in this respect. Covalent binding of the receptor to the ligand is rather disadvantageous due to the generally considerable activation energy and the resultant decrease in specificity of the interaction.

According to the invention, the ligands are immobilized by means of anchors on the sensing surface of the sensor. An anchor molecule according to the invention comprises at least two functional moieties at opposite ends of the anchor which enable attachment to the sensor surface on the one hand and binding of the ligand on the other hand. Moreover, if solid-phase synthesis is applied, it should be possible to link the basic units of the anchor to the solid phase used for synthesis and to break this bond after successful LAC synthesis under mild conditions. Mild conditions are conditions which do not affect the properties of the LAC which are essential for providing the biospecific boundary layer. The bond between anchor and solid phase during the LAC synthesis is preferably covalent.

It is a specific object of the invention to use synthesis methods, e.g. of combinatorial chemistry, for generating bioactive surfaces on the basis of organic monolayers. However, for the reasons mentioned above, the synthesis is not to be carried out directly on the monolayer. Combinatorial chemistry offers various techniques which are capable of producing a multitude of different substances (so-called substance libraries) in few, often automated reaction sequences (cf. e.g. M. A. Gallop et al., J. Med. Chem. 37 (1994) 1233-1251, E. M. Gorden et al., J. Med. Chem. 37 (1994) 1385-1401). Here, the reactions are preferably also carried out on the solid phase for practical reasons. The carrier materials are usually cross-linked polymers in form of particles (so-called polystyrene or polyethylene glycol/polystyrene resin beads). Based on a functionalized surface, the desired structures are prepared in several synthesis steps. L. A. Thompson and J. A. Ellman, Chem. Rev. 96 (1996) 555-600 give an overview of the synthesis of substance libraries on solids as well as in liquid phase. After termination of the combinatorial solid-phase synthesis, the products are generally cleaved from the solid phase, i.e. they are released by cleavage of an unstable bond between end product and carrier resin. Subsequently, they are purified for the purpose of HTS or directly transferred in biological assay media. It has also been attempted to leave the products on the beads and carry out the biomolecular interaction studies directly on the carrier material. This, however, entails considerable disadvantages, since the substrate materials suitable for the organochemical synthesis are not suitable for interaction analyses on account of their high unspecific binding capacities.

The synthesis of a library of LACs which differ only in their ligands is considerably facilitated by the use of a prefabricated solid phase which already comprises the anchor. In this case, the particles pre-modified for the anchor-conjugate synthesis are already provided with all molecular elements necessary for the stepwise formation or attachment of the ligands, including the entire anchor molecule. The anchor is coupled to the solid phase via a linker which permits LAC release, after synthesis, preferably under mild conditions.

In simple coupling reactions, aliquots of the pre-modified solid phase may then be provided with a multitude of different ligands. During the cleavage from the carrier material, the conjugated coating structures (LACs) are released and the monolayers assemble automatically when the LACs are applied to the surface of the sensor (contacting).

Consequently, the present invention provides a binding matrix with a defined surface by means of simple chemical synthesis, which offers high flexibility as regards the selection and possibilities of chemical modifications of the immobilized ligands and may suitably be used in methods with high sample throughput (HTS).

Metals, noble metals or metal oxides or composite materials onto whose surfaces noble metals, metals such as copper or metal oxides are applied are preferred as carrier materials onto which the anchor-ligand conjugates or mixtures thereof are applied for providing the sensor. Particularly preferred are noble metals, such as silver, gold, palladium or platinum and most preferred is gold.

According to a further embodiment of the invention, biospecific boundary layers may also be provided on plastics. The surfaces of commercially available polymer materials, such as polyalkylenes (such as PP or PE), PTFE, PMMA or polycarbonates may be used as well as polymer mixtures comprising one or more of these polymers. Furthermore, copolymers of such monomers forming the aforementioned plastics materials may be applied.

The anchors immobilized on the substrate in the ready-for-use measuring arrangement should have structural subunits fulfilling the following tasks:
a) immobilisation of the anchor on the sensor surface;
b) binding of the ligand L or its generation on the anchor or the part of the anchor opposite the sensor surface (in ω position);
c) formation of a monolayer (self-assembled monolayer, SAM) if the LACs are contacted with the sensor surface.

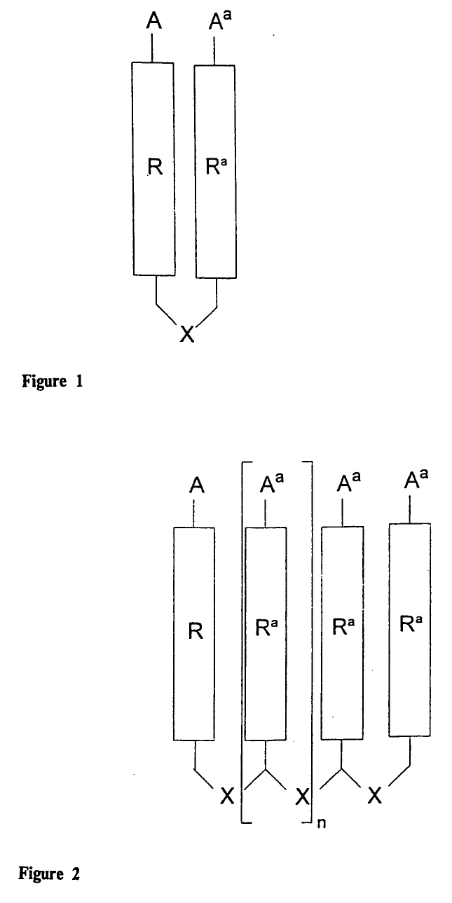

FIG. 1 represents a schematic view of the design of an anchor molecule according to the invention:

The structural component X permits immobilisation of the ready-to-use LACs on the sensor surface, the groups R and R^{a} represent residues allowing for an SAM formation. The terminal groups A and A^{a} serve for binding ligands or non-ligands. R^{a} and A^{a} combined or A^{a} alone may optionally be replaced by a hydrogen atom. The shown structural subunits of the anchor are covalently linked, either directly or via short-chain bivalent coupling groups, such as C₁-C₄ alkylene, in particular methylene or ethylene (symbolized by lines in the schematic view). The anchor may additionally comprise a structural unit Y which originates from the linker for attachment to the solid phase during the synthesis.

The group X serves for immobilizing the LACs on the sensor surface and preferably comprises an element of any of main group V or VI of the periodic table, including combinations of identical or different elements. Combinations of such elements, e.g. —S—Se— or —Se—Se— are advantageous. Depending on the surface quality, the use of groups that are ionized at neutral pH, such as sulfonate, is advantageous. Sulphur is preferably present, e.g. in form of the disulfide function (—S—S—), the thiol function (—SH) or the sulfide function (—S—). The elements used are characterized by either a high affinity for metals, in particular noble metals (gold, silver etc.) and thus allow an immobilisation of the ligand-anchor conjugates, for example on a gold, silver or platinum surface, or their capability for the attachment to a metal oxide surface, such as Al₂O₃, if an ionic group is chosen.

It is known that, beside thiols, sulfides are also particularly useful for forming SAMs (Troughton et al., Langmuir 4 (1988) 365-85; Schierbaum et al., Science 265 (1994) 1413-5; Huismann et al., JACS 118 (1996) 3523-4). As regards stability, sulfides have advantages over thiols or disulfides in chemical synthesis, in particular solid-phase synthesis.

A particularly preferred embodiment of the present invention therefore consists in anchor molecules which are immobilized on the sensor surface on the basis of a sulfide group. On an c position of the chain facing away from the sulphur, the “sulfide anchors” may be provided with molecular structures of different functionality. It is of particular advantage to carry out the binding of the structures before the adsorption, thus allowing complete analytical characterization of the obtained conjugate before immobilisation and the examination of its structural integrity. Since sulphur-gold complexation is one of the few methods of non-covalent surface modification, the assumption is justified that the chemical structure of the conjugates is not changed by the adsorption process. By using such functionalized conjugates, any further surface modification which cannot be detected in chemical analysis is avoided. Thus, only one single coating step is necessary for providing surfaces with biospecific boundary layers, even with complex structures.

The chemical nature of the used group X simultaneously determines the basic chemical structure of the anchor. By using thiols, LACs are produced which have only one single chain that may optionally be branched. In contrast, the use of sulfides and disulfides makes anchors available that are exemplarily shown in FIG. 1; they comprise two chain structures separated from each other by the group X.

If the LACs according to the invention are applied to plastics surfaces, the aforementioned groups X most of all serve for structuring the anchor molecule, whereas group R or groups R and R^{a} allow for attractive interactions with the polymer surface.

R and R^{a} may be the same or different and represent a branched or unbranched, optionally substituted, saturated or unsaturated hydrocarbon chain which may be interrupted by heteroatoms, aromatic and heterocyclic subunits and comprises 2-2000 atoms, including heteroatoms. If X-type structural units are e.g. linked with each other by the use of polyvalent residues R or R^{a}, oligomeric LACs may also be prepared; the latter are schematically shown in FIG. 2, below, wherein n is an integer ≧0. R^{a} and A^{a} are defined as above and can both also be the same or different.

The anchors according to the invention are functionalized in ω position relative to the group X in order to permit binding of the ligands. Functional groups A or A^{a} which may be used for this purpose are i.a.: acetals, ketals, acylals, acid halides, alcohols (hydroxy groups), aldehydes, alkenes, halides, alkines, allenes, amides, amidines, aminals, amines, anhydrides, azides, azines, aziridines, azo compounds, boranes, carbamates, carbodiimides, carboxylic acids, carbonic esters, cyanamides, cyanates, diazo compounds, diazonium salts, epoxides, ethers, hydrazides, hydrazines, hydrazones, hydroxamic acids, hydroxamic esters, hydroxyl amines, imides, imines, inorganic esters, isocyanates, isocyanides, isothiocyanates, ketenes, ketones, nitriles, nitro compounds, nitroso compounds, oximes, phenols, phosphines, phosphonates, ammonium salts, phosphonium salts, sulfonamides, sulfones, sulfonic acids, sulfone esters, sulfonium salts, sulfonyl azides, sulfonyl halides, sulfoxides, thioamides, thiocarbamates, thiocyanates, triazenes, ureas or isoureas. The residues A and A^{a} may be the same or different and A^{a} may be replaced by a hydrogen atom. They should preferably comprise less than 10, more preferably less than 4 C atoms. Functional groups A and A^{a} are most preferably hydroxyl groups, primary or secondary amines, preferably C₁-C₄ N-alkylated, and carboxylic acids directly connected with R or R^{a} as substituents. They may optionally be activated (e.g. as active esters) in order to facilitate binding of the ligands.

Reactions for binding the ligand to the anchor may e.g. be substitution or addition reactions, elimination reactions (addition elimination reactions, such as condensation reactions), reactions for establishing double bonds, such as the Wittig reaction, or a C—C single bond, such as the Aldol, Heck or Suzuki reaction, or electrocyclic reactions. This list is not exhaustive or restrictive and may easily be completed by a skilled person.

The method according to the invention is advantageous in that for binding the ligand to the anchor and for attachment to the solid phase (SP) the same functional group may be used, since the latter is regioselectively “blocked” by the solid phase when the ligand is bound.

The anchor structure may comprise the following functional groups (Y) on account of its binding to a solid phase during the synthesis even after separation of the LACs from the linker: acetals, ketals, acylals, acid halides, alcohols, aldehydes, alkenes, halides, alkines, allenes, amides, amidines, aminals, amines, anhydrides, azides, azines, aziridines, azo compounds, boranes, carbamates, carbodiimides, carboxylic acids, carbonic esters, cyanamides, cyanates, diazo compounds, diazonium salts, epoxides, ethers, hydrazides, hydrazines, hydrazones, hydroxamic acids, hydroxamic esters, hydroxylamines, imides, imines, inorganic esters, isocyanates, isocyanides, isothiocyanates, ketenes, ketones, nitriles, nitro compounds, nitroso compounds, oximes, phenols, phosphines, phosphonates, ammonium salts, phosphonium salts, sulfonamides, sulfones, sulfonic acids, sulfone esters, sulfonium salts, sulfonyl azides, sulfonyl halides, sulfoxides, thioamides, thiocarbamates, thiocyanates, triazenes, ureas or isoureas. These residues are preferably directly linked with the residue of the anchor or via a side chain optionally contained in Y, which side chain comprises 1-8, preferably 1-4 C atoms. It may be interrupted by further functional units, in particular —O— or —CONH—. Residue Y as a whole comprises ≦20, preferably ≦10, more preferably ≦5 C atoms.

If specific linker compounds are used for attaching the anchor to the solid phase (“traceless linker”), the LAC may also be cleaved without a functional group remaining on the anchor structure. In this case, the linker is replaced by a hydrogen atom during cleavage. Surprisingly, it has been found that despite the presence of a functional group Y in one arm of the anchor, the formation of SAMS is not or not significantly affected. Therefore, conventional and often less expensive linkers may be used that leave such groups.

Linkers which may advantageously be used in solid-phase synthesis and groups remaining on the target molecule after separation of the latter from the solid phase, are described e.g. in Novabiochem® Combinatorial Chemistry Catalog & Solid Phase Organic Chemistry Handbook, March 98, Callbiochem-Novobiochem AG, Switzerland. Despite the use of identical linkers, the structure of the resulting group Y may vary depending on the used cleavage reagent. Preferred groups which remain on the anchor after separation of the LACs from the solid phase are listed in the following Table:

| | Y after separation from the solid phase |
|---|---|
| Y bound to SP | (SP) |
| —CO— | —COOH |
| | —COOR′ |
| | —CHO |
| | —CH₂OH |
| | —CO—NR′₂ |
| | —CO—NH—OH |
| | —CO—NH—NH₂ |
| | -cyclo[CO—NH—CH(R′)—CO—NH—CH] |
| —O— | —OH |

wherein each of the residues R′ may independently be a hydrogen atom or an alkyl group, preferably a hydrogen atom or a C₁-C₄ alkyl group. If there is a group Y, —CONR′₂, —COOH or —OH are particularly preferred.

The above preferred and particularly preferred groups Y can additionally comprise a coupling group at the free valency; by means of this coupling group, they are linked to the residue of the anchor structure, preferably via R or R^{a}. The coupling group is preferably an at least bivalent organic residue that may be unbranched or branched and preferably comprises 1-8, particularly preferably 1-4 carbon atoms. It may be interrupted by additional functional groups, in particular —O—, —CONR′—, wherein R′ is defined as mentioned above. C1-C4 alkylene groups, such as methylene, ethylene or propylene, are particularly preferred.

In a further preferred embodiment, the aforementioned free valency is directly connected to R or R^{a}.

Preferably, the anchor comprises structures which make difficult or avoid a passive adsorption of the receptor, both at the anchor structure and the sensor surface. Moreover, it is advantageous that the anchor comprises a spacer group which enables the adaptation of the length of the entire chain and the LAC flexibility.

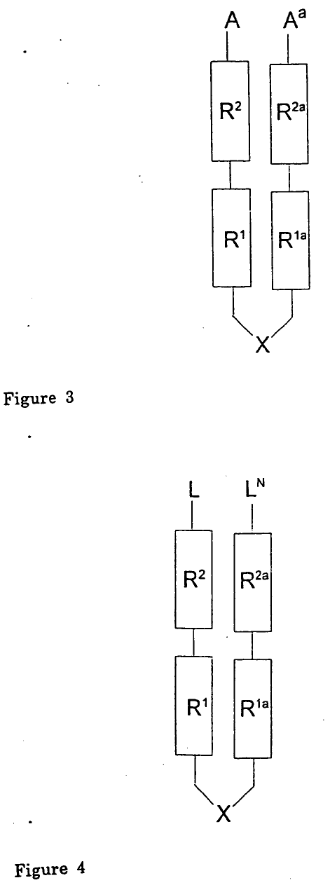

Therefore, a preferred embodiment of the anchor according to the invention is the structure schematically shown in FIG. 3 wherein A, A^{a} and X are defined as above, and R¹ and R² or R^{1a} and R^{2a}, respectively, form the residue R or R^{a}. The residue Y, if present, can preferably be bound to R¹ or R^{1a} or to R¹ or R^{2a} as a side chain. In a particularly preferred embodiment, it is therefore near or at the point of linkage between R¹ or R^{1a} and R¹ or R^{2a}, respectively.

Preferably, R¹ and R^{1a} serve to generate an SAM and should be largely hydrophobic for this purpose. They independently comprise a branched or unbranched hydrocarbon chain with 1 to 50 C atoms which may be completely saturated or partly unsaturated and interrupted by aromatic or heterocyclic subunits or heteroatoms, a hydrocarbon chain without heteroatoms being preferred. Preferably, it has the general formula —(CH₂)ₙ—, n being an integer between 1 and 50, preferably 3 and 25, more preferably 4 and 18 and most preferably 8 to 12.

For the introduction of R¹ and/or R^{1a} in a particularly preferred form, commercially available compounds may be used, in particular functionalized alkanes bearing functional groups, such as e.g. hydroxyl groups, halogen atoms, carbonic acid groups or mercapto groups, at both terminal groups. These terminal functional groups e.g. facilitate binding to the adjacent structural groups during anchor synthesis. Optionally, they assist in introducing necessary anchor components, such as X. Exemplarily, 11-bromo-1-undecanol, 1,10-decandiol or 11-mercaptoundecanoic acid are listed. The latter simultaneously guarantees the introduction of the sulphur function as (X).

R² and R^{2a} are preferably spacers enabling the adaptation of the entire chain length and the flexibility of the ligand-anchor conjugate. Preferably, they independently represent hydrocarbon chains which are interrupted by heteroatoms and are therefore hydrophilic and preferably make difficult a passive adsorption of the receptor. The chain comprises 2 to 1000 atoms, including heteroatoms. Chain lengths of 5 to 500 are preferred and chain lengths of 10 to 100 atoms are particularly preferred.

In a preferred embodiment, R² and/or R^{2a} are/is an oligoether of the general formula —(OAlk)_{y}—, wherein y is an integer and Alk represents an alkylene group. Preferred is a structure in which y is between 1 and 100, preferably between 1 and 20 and most preferably between 2 and 10. The residue Alk has preferably 1-20, particularly preferably 2-10, and more preferably 2-5 C atoms. —(OC₂H₄)_{y}— is most preferred.

In a second preferred embodiment, R² and/or R^{2a} are/is an oligoamide of dicarboxylic acids and diamines and/or amino acids, wherein the amines comprise independently preferably between 1 and 20, particularly preferably 1 to 10 carbon atoms and may also be interrupted by further heteroatoms, in particular oxygen atoms. The carboxylic acid monomers comprise independently preferably 1 to 20, particularly preferably 1 to 10 carbon atoms and may also be interrupted by further heteroatoms, in particular oxygen atoms.

In a particularly preferred embodiment, commercially available compounds, such as in particular glycol ether, such as e.g. triethylene glycol, triethylene glycol monomethyl ether, tetraethylene glycol, as well as dicarboxylic acids, such as succinic acid, 1,13-diamino-4,7,10-trioxatridecane, 3,6,9-trioxaundecanediacid, 8-amino-3,6-dioxaoctanoic acid or 4-aminobenzoic acid as well as their derivatives or combinations of identical structural elements (such as e.g. in 8-amino-3,6-dioxaoctanoic acid or 4-aminobenzoic acid) or combinations of different structural units (such as e.g. 1,13-diamino-4,7,10-trioxatridecane and 3,6,9-trioxaundecanediacid in alternating sequence) are used to generate R² and/or R^{2a}. One advantage of using 4-aminobenzoic acid is its good spectroscopic detectability, e.g. by means of ultraviolet spectroscopy.

Whereas R¹ and R² must be present in the structural formula of FIG. 3, one or two optional structural units, selected from R^{1a}, R^{2a} and F^{a} may optionally be missing. Optionally, the combination of R^{1a}, R^{3a} and A^{a} may also be completely replaced by a hydrogen atom.

In a particularly preferred embodiment of the present invention, the anchor structures 1 to 16 are provided as illustrated below.

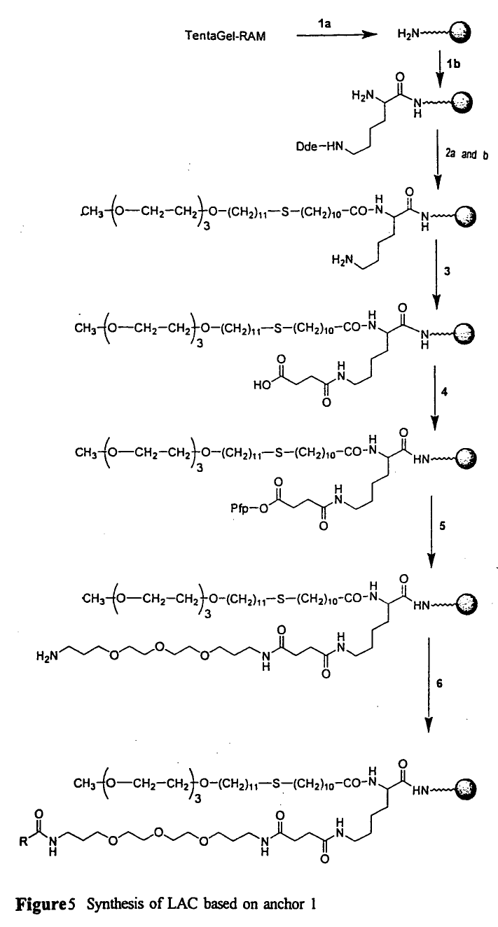

In a preferred anchor form, which comprises two arms, both of the terminal groups A and A^{a} are provided. For preparing the LACs, they may both be occupied by ligands (L) which are capable of interacting with the receptor which may be attached to both A and A^{a}. Preferably, only one terminal group is occupied by a ligand whereas the other one is capped with a low molecular weight compound (L^{N}) which is not capable of such interactions (non-ligand). FIG. 4 shows the corresponding structure.

Such SAM-forming LAC substructures, which, equipped with an L^{N} group, are no longer available for detection purposes, allow for an accurate structuring of potentially interacting ligands on the sensor surface, while simultaneously a passive, i.e. unspecific adsorption of the receptor in the formed gaps is avoided. They are termed “diluting components.” For intensifying this effect, anchor structures which exclusively bear L^{N} groups may optionally additionally be used during the provision of the sensor surface.

The ligand L serves for providing specific structural features in the formed boundary layer which is thus available for interacting with the receptor. In the course of solid-phase synthesis, ligand L may be bound to the anchor in a single step or prepared in several synthesis steps on the anchor. The latter method is particularly advantageous if combinatorial synthesis methods are applied in which a large number of structurally diverse ligands can be produced in few steps, using mixtures of two or more synthetic structural units. Ligands which are typically used for providing the sensor surface with bioactivity are: proteins, peptides, oligonucleotides, carbohydrates (glycosides), isoprenoids, enzymes, lipid structures as well as organic molecules which have a molecular weight of ≧50 g/mol and have characteristic spatial or electronic structures, such as e.g. an aminoacid, a nucleoside, a heterocyclic compound, an alycyclic compound, an aromatic compound, a terpene, an organophosphorus compound, a chelate complex, a neurotransmitter, a substituted amine, an alcohol, an ester, an ether or a carboxylic acid and its derivatives. They can be synthesized by using reactions known from the literature (cf. e.g. J. S. Früchtel, G. Jung, Angew. Chem. Int. Ed. 35 (1996) 17-42). This list is neither exhaustive nor restrictive and may easily be supplemented by the skilled person.

WO-A2-8903041 und WO-A1-8903042 describe molecules having molecular weights of up to 7000 g/mol as small molecules. Usually, however, the molecular weights are stated to be between 50 and 3000 g/mol, more commonly between 75 and 2000 g/mol and most usually within the range of from 100 to 1000 g/mol. Such small molecules are, e.g., disclosed in WO-A1-8602736, WO-A1-9731269, U.S. Pat. No. 5,928,868, U.S. Pat. No. 5,242,902, U.S. Pat. No. 5,468,651, U.S. Pat. No. 5,547,853, U.S. Pat. No. 5,616,562, U.S. Pat. No. 5,641,690, U.S. Pat. No. 4,956,303 and U.S. Pat. No. 5,928,643.

Within the scope of the present invention, the molecular weight of a ligand/small molecule (without anchor) is to be between 50 and 500 g/mol, preferably between 75 and 1500 g/mol. The following compounds are examples of small molecules which can be used as ligands within the scope of the present invention:

Propargylamine, cyclopropylamine, propylamine, ethylenediamine, ethanolamine, imidazole, 3-aminopropionitrile, pyrrolidine, glyoxylic acid monohydrate, acetic hydrazide, I-glycine, glycolic acid, pyridine, 1-methylimidazol, cyanoacetic acid, cyclopropanecarboxylic acid, (s)-(+)-3-methyl-2-butylamine, pyruvic acid, n,n-dimethylethylenediamine, n,n′-dimethylethylenediamine, 1-alanine, beta-1-alanine, d-alanine, beta-alanine, sarcosine, (r)-2-amino-1-butanol, 2-amino-1,3-propanediol, aniline, 3-aminopyridine, 4-pentynoic acid, 4-pentenoic acid, alpha-beta-dehyro-2-aminobutyric acid, aminocyclopropylcarboxylic acid, 3-amino-1-propanol vinyl ether, (r)-(−)-tetrahydrofurfurylamine, (s)-(+)-prolinol, (r)-3,3-dimethyl-2-butylamine, 1,5-diaminopentane, gamma-aminobutyric acid, 2-aminobutyric acid, 2-aminoisobutyric acid, 3-amino-2,2-dimethyl-1-propanol, thiomorpholine, 1-2,3-diaminopropionic acid, d-serine, 1-serine, 2-(2-aminoethoxy)ethanol, (methylthio)acetic acid, benzylamine, 3-chloropropionic acid, 4-aminophenol, histamine, quinuclidine, exo-2-aminonorbornane, cyclopentanecarboxylic acid, trans-1,4-diaminocyclohexane, 1-proline, d-proline, 1-allylglycine, 1-amino-1-cyclopentanemethanol, tetrahydro-2-furoic acid, 3,3-dimethylbutyric acid, succinamic acid, 1-valine, 1-leucinol, hydantoic acid, 1-threonine, d-threonine, (s)-(−)-alpha-methylbenzylamine, 2-(2-aminoethyl)pyridine, 5-amino-o-cresol, p-anisidine, pyrazinecarboxylic acid, 1-(3-aminopropyl)imidazole, tropane, cyclooctylamine, 1-alpha-aminocaprolactam, 5-oxo-1-proline, isonipecotic acid, 1-pipecolic acid, 1,4,7-triazacyclononane, octylamine, dibutylamine, 4-methyl-2-oxovaleric acid, 1-aspartic acid, 1-asparagine, 1-leucine, 6-aminohexanoic acid, 1-isoleucine, 1-alpha-t-butylglycine, d-leucine, z-beta-alanine, 1-asparagine, 1-ornithine, 5-aminoindole, 1-aspartic acid, d-aspartic acid, 1-thiazolidine-4-carboxylic acid, 4-aminobenzoic acid, 3-(2-furyl)acrylic acid, 3-thiopheneacetic acid, cycloheptanecarboxylic acid, 3,5-difluorobenzylamine, 1,4-dioxa-8-azaspiro[4,5]-decane, n-cyclohexylethanolamine, caprylic acid, 1-glutamine, d-glutamine, 1-lysine, d-glutamic acid, 1-glutamic acid, 4-cyanobenzoic acid, (s)-1,2,3,4-tetrahydro-1-naphthylamine, 2,2,3,3,3-pentafluoropropylamine, (1s,2r)-(−)-cis-1-amino-2-indanol, 1-methionine, d-methionine, 4-carboxybenzaldehyde, 3-phenylpropionic acid, 4′-aminoacetanilide, piperonylamine, 1-phenylglycine, d-phenylglycine, 4-(aminomethyl)benzoic acid, 1-adamantanamine, 4-(hydroxymethyl)benzoic acid, (−)-cis-myrtanylamine, (1r,2r,3r,5s)-(−)-isopinocampheylamine, (r)-(+)-bornylamine, 1,3,3-trimethyl-6-azabicyclo[3,2,1]octane, 3,5-dihydroxybenzoic acid, 2-norbornaneacetic acid, 1-2-furylalanine, 1-histidine, d-histidine, 1-cyclohexylglycine, ethyl pipecolinate, 5-amino-1-naphthol, tryptamine, 4-aminobutyraldehyde diethyl acetal, 2-benzofurancarboxylic acid, 1-indoline-2-carboxylic acid, d-phenylalanine, 1-phenylalanine, 4-dimethylaminobenzoic acid, 1-methionine-sulfoxide, 3-(4-hydroxyphenyl)-propionic acid, dl-atrolactic acid hemihydrate, 4-sulfamoylbutyric acid, vanillic acid, 4-aminobiphenyl, (r)-(+)-citronellic acid, 4-chlorophenylacetic acid, 1-3-thienylalanine, 1-cyclohexylalanine, d-cyclohexylalanine, (s)-(−)-1-(1-naphthyl)-ethylamine, 2-chloro-6-methylnicotinic acid, 1-arginine, d-arginine, 14-thiazolylalanine, 3-pyridylacetic acid hydrochloride, 3-indolylacetic acid, 7-amino-4-methylcoumarin, 1-citrulline, 4-benzylpiperidine, 2,4-dichlorobenzylamine, 4-amino-n-methylphthalimide, (−)-cotinine, 1-tetrahydroisoquinolinecarboxylic acid, 4-acetamidobenzoic acid, (r)-(−)-2-benzylamino-1-butanol, 4-pentyloxyaniline, o-acetylsalicylic acid, 4-nitrophenylacetic acid, 2-nitrophenylacetic acid, 2-methyl-6-nitrobenzoic acid, 1-tyrosine, d-tyrosine, 1-methionine(o2), 3-(diethylamino)propionic acid hydrochloride, 4-nitroanthranilic acid, 2,6-dimethoxybenzoic acid, 3,5-dimethoxybenzoic acid, 3,4-dihydroxyhydrocinnamic acid, 2-(4-hydroxyphenoxy)propionic acid, 2-methoxyphenoxyacetic acid, 4-hydroxy-3-methoxyphenylacetic acid, 4-(ethylthio)benzoic acid, s-benzylthioglycolic acid, 4-(methylthio)phenylacetic acid, 2-chlorocinnamic acid, 3-chlorocinnamic acid, gamma-maleimidobutyric acid, 2,6-dimethoxynicotinic acid, 1-4-fluorophenylalanine, 1-2-fluorophenylalanine, (r)-(−)-epinephrine, cyclododecylamine, trans-2,5-difluorocinnamic acid, dl-3,4-dihydroxymandelic acid, thymine-1-acetic acid, cis-pinonic acid, 1,2-bis(4-pyridyl)ethane, 4-tert-butylcyclohexanecarboxylic acid, n,n-diethylnipecotamide, 3,4-difluorohydrocinnamic acid, 2-naphthylacetic acid, 3-carboxy-proxyl, 4-chloro-o-anisic acid, 4-chlorophenoxyacetic acid, 3-chloro-4-hydroxyphenylacetic acid, 5-chloro-2-methoxybenzoic acid, 4-chloro-dl-mandelic acid, 4-(pyrrol-1-yl)benzoic acid, 4-(difluoromethoxy)benzoic acid, gallic acid monohydrate, 2,4,6-trihydroxybenzoic acid monohydrate, 6-hydroxy-2-naphthoic acid, suberic acid monomethyl ester, 2-hydroxydecanoic acid, 2-chloro-6-fluorophenylacetic acid, alpha-cyano-3-hydroxycinnamic acid, indole-3-glyoxylic acid, 8-hydroxyquinoline-2-carboxylic acid, 2-methyl-3-indoleacetic acid, 4-(trifluoromethyl)benzoic acid, coumarin-3-carboxylic acid, 3-hydroxy-2-quinoxalinecarboxylic acid, 4-fluoro-1-naphthoic acid, 1-phenyl-1-cyclopentanecarboxylic acid, p-toluenesulonyl chloride, 5-bromo-2-furoic acid, 2,5-dichlorobenzoic acid, 3,4-dichlorobenzoic acid, 5-methoxyindole-2-carboxylic acid, isoquinoline-3-carboxylic acid hydrate, 1-styrylalanine, 4-(dimethylamino)cinnamic acid, 4-oxo-2-thioxo-3-thiazolidinylacetic acid, 1-ethyl-3-(3-dimethylaminopropyl)carbodiimide hydrochloride, 5,6-dichloronicotinic acid, 2,6-dichloronicotinic acid, 2,6-dichloropyridine-4-carboxylic acid, trimellitic anhydride, d-(−)-quinic acid, trans-3,4-methylenedioxycinnamic acid, 7-methoxybenzofuran-2-carboxylic acid, trans-5-acetoxy-1,3-oxathiolane-2-carboxylic acid, 4-benzoylbutyric acid, 4-pentylbenzoic acid, 6-phenylhexanoic acid, 2-chloro-4,5-difluorobenzoic acid, 4-chloro-2,5-difluorobenzoic acid, 5-fluoroindole-3-acetic acid, n-formyl-dl-phenylalanine, 4-diethylaminobenzoic acid, 2-aminoanthracene, d-glucuronic acid, trans-ferulic acid, (s)-(+)-o-acetylmandelic acid, 4-aminohippuric acid, 1-adamantaneacetic acid, 6-bromohexanoic acid, alpha-hydroxyhippuric acid, n-[3-(2-furylacryloyl)]-glycine, 1-methyl 2-aminoterephthalate, 1-serine(bzl), 3,3,3-trifluoro-2-(trifluoromethyl)propionic acid, diethylphosphonoacetic acid, d-gluconic acid, 3-(4-fluorobenzoyl)propionic acid, 2,5-dimethoxyphenylacetic acid, mono-methyl cis-5-norbornene-endo-2,3-dicarboxylate, 4-hydroxy-3-nitrophenylacetic acid, 3-methoxy-4-nitrobenzoic acid, 5-methoxy-2-nitrobenzoic acid, 3,4,5-trimethoxybenzylamine, dl-4-hydroxy-3-methoxymandelic acid, (−)-camphanic acid, (1r)-(+)-camphanic acid, 2-methoxy-4-(methylthio)benzoic acid, cis-5-dodecenoic acid, 4-amino-5-carboxy-2-ethyl-mercaptopyrimidine, 4-aminocinnamic acid hydrochloride, dl-3-(4-hydroxyphenyl)lactic acid hydrate, 4-(methylsulfonyl)benzoic acid, 4-carboxy-2,2,6,6-tetramethylpiperidine 1-oxyl, 2-butyloctanoic acid, trans-2-chloro-6-fluorocinnamic acid, 4-chloro-o-tolyloxyacetic acid, 2-bromobenzoic acid, 4-carboxybenzenesulfonamide, 2-(2-aminothiazole-4-yl)-2-methoxyiminoacetic acid, 1-(n-t-amino)-cyclopropanecarboxylic acid, 2-chloro-3-nitrobenzoic acid, 4-chloro-3-nitrobenzoic acid, 2-chloro-4-nitrobenzoic acid, 4-chloro-2-nitrobenzoic acid, 4-amino-5-chloro-2-methoxybenzoic acid, 5-bromonicotinic acid, 6-bromopicolinic acid, 2-methyl-5-phenylfuran-3-carboxylic acid, tributyl phosphine, 2-chloro-5-(methylthio)benzoic acid, 4,5-difluoro-2-nitrobenzoic acid, 2-hydroxy-5-(pyrrol-1-yl)benzoic acid, indole-3-butyric acid, 2-(trifluoromethyl)phenylacetic acid, 3-(trifluoromethyl)phenylacetic acid, 4-(trifluoromethyl)phenylacetic acid, 3,7-dihydroxy-2-naphthoic acid, 6-methylchromone-2-carboxylic acid, 1-tryptophan, d-tryptophan, 2,6-dichlorophenylacetic acid, 3,4-dichlorophenylacetic acid, 3-(trifluoromethyl)anthranilic acid, alpha-acetamidocinnamic acid, 5-methoxyindole-3-acetic acid, dl-indole-3-lactic acid, (1s,2s)-(−)-2-benzyloxycyclohexylamine, 3,5-dichloroanthranilic acid, chloramben, s-(+)-ibuprofen, dl-thioctic acid, 3,5-dichloro-4-hydroxybenzoic acid, 5-bromothiophene-2-carboxylic acid, 2,3,5,6-tetrafluoro-p-toluic acid, 2-fluoro-3-(trifluoromethyl)benzoic acid, 3-fluoro-4-(trifluoromethyl)benzoic acid, 5-azido-2-nitrobenzoic acid, trans-2,3-dimethoxycinnamic acid, n-(4-aminobenzoyl)-beta-alanine, 4-butoxyphenylacetic acid, 2-(2-aminophenyl)indole, 2-amino-3,4,5,6-tetrafluorobenzoic acid, 2-nitrophenylpyruvic acid, z-glycine, 4-(4-nitrophenyl)butyric acid, s-(−)-2-[(phenylamino)carbonyloxy]propionic acid, 1-threonine(bzl), 2,6-dichloro-5-fluoro-3-pyridinecarboxylic acid, trimesic acid, (4-formyl-3-methoxy-phenoxy)acetic acid, (e)-5-(2-carboxyvinyl)-2,4-dimethoxypyrimidine, 1-phenylalanine(4-no2), 2-oxo-6-pentyl-2h-pyran-3-carboxylic acid, n,n-bis(2-hydroxyethyl)-isonicotinamide, (+/−)-jasmonic acid, epsilon-maleimidocaproic acid, (s)-(−)-n-benzyl-1-phenylethylamine, 2,4-dinitrobenzoic acid, 2,4,5-trimethoxybenzoic acid, 3,4,5-trimethoxybenzoic acid, s-(thiobenzoyl)thioglycolic acid, 4-iodobutyric acid, 3-phenoxybenzoic acid, 4-(4-hydroxyphenyl)benzoic acid, d-desthiobiotin, (−)-menthoxyacetic acid, 2-(o-chlorophenoxy)-2-methyl-propionic acid, 4-bromophenylacetic acid, 3-bromo-4-methylbenzoic acid, 3-bromophenylacetic acid, [1 r-(1alpha,2beta,3alpha)]-(+)-3-methyl-2-(nitromethyl)-5-oxocyclopentaneacetic acid, 1-aspartic acid(ochx), 1-1-naphthylalanine, 2-(trifluoromethyl)cinnamic acid, monomethyl sebacate, 5-aminovaleric acid, o-carboxyphenyl phosphate, 4-(trifluoromethyl)hydrocinnamic acid, mono-ethyl(r)-3-acetoxyglutarate, beta-(naphthylmercapto)acetic acid, 3-bromo-4-fluorobenzoic acid, 3-phthalimido-propionic acid, 1-arginine(no2), cis-(1s,2r)-(−)-2-benzylaminocyclohexanemethanol, 7-hydroxycoumarin-4-acetic acid, 2-sulfobenzoic acid hydrate, 5-methoxy-1-indanone-3-acetic acid, 4,7,10-trioxa-1,13-tridecanediamine, 2,4-dichlorophenoxyacetic acid, (s)-(+)-2-oxo-4-phenyl-3-oxazolidineacetic acid, (s)-(−)-n-(1-phenylethyl)succinamic acid, 3-(trifluoromethylthio)benzoic acid, 5-(4-chlorophenyl)-2-furoic acid, 8-bromooctanoic acid, 1-aspartic acid(obzl), n-acetyl-1-tyrosine, 2-nitro-5-thiocyanatobenzoic acid, 9-fluorenone-4-carboxylic acid, fluorene-9-acetic acid, 2-chloro-5-(trifluoromethyl)benzoic acid, 1-(4-chlorophenyl)-1-cyclopentanecarboxylic acid, 3,5-diaminobenzoic acid dihydrochloride, n-acetyl-4-fluoro-dl-phenylalanine, 2,4,6-trichlorobenzoic acid, 2,3,4,5,6-pentafluorophenylacetic acid, 2,4-dinitrophenylacetic acid, 3,4,5-trimethoxyphenylacetic acid, xanthene-9-carboxylic acid, (r)-(+)-3-hydroxy-5-oxo-1-cyclopentene-1-heptanoic acid, 2-bibenzylcarboxylic acid, 2,2-diphenylpropionic acid, 4-bromocinnamic acid, 4-carboxybenzenesulfonazide, 3-benzoyl-2-pyridinecarboxylic acid, trans-4-chloro-3-nitrocinnamic acid, 2,3,5,6-tetrafluoro-4-hydroxybenzoic acid hydrate, 3,5-dinitrosalicylic acid, (z)-(2-(formamido)thiazol-4-yl)(methoxyimino)acetic acid, 1-glutamic acid gamma-cyclohexyl ester, mono-2-(methacryloyloxy)ethyl succinate, naproxen, 1-lysine(alloc)-oh, 4-bromomandelic acid, 2-bromo-5-methoxybenzoic acid, 1-hydroxyproline, 6-(amino)-hexanoic acid, n-tert-butoxycarbonyl-1-leucine, 4-bromo-3,5-dihydroxybenzoic acid, n-(4-carboxy-3-hydroxyphenyl)maleimide, 5-(2-nitrophenyl)-2-furoic acid, 5-(3-nitrophenyl)-2-furoic acid, n-phthaloyl-dl-alpha-aminobutyric acid, 1-thiazolidine-4-carboxylic acid, (s)-(−)-alpha-methoxy-alpha-(trifluoromethyl)phenylacetic acid, 7-carboxymethoxy-4-methylcoumarin, 3,5-di-tert-butylbenzoic acid, 2-(2-chloroacetamido)4-thiazoleacetic acid, 5-bromoorotic acid, 2-nitro-alpha,alpha,alpha-trifluoro-p-toluic acid, benzoyl-dl-leucine, 1-glutamic acid(obzl), n,n′-dibenzylethylenediamine, 1-biphenylalanine, diphenic acid, 1-4-bromophenylalanine, pindolol, 1-leucine-4-nitroanilide, alpha, alpha-diphenyl-1-prolinol, 1-pentafluorophenylalanine, 1-phosphotyrosine, 4-iodophenylacetic acid, 1-benzoylphenylalanine, methyl red, 1-tyrosine(bzl), pentafluorophenyl trifluoroacetate, 1-lysine(z), r-(+)-1,1′-binaphtyl-2,2′-diamine, (+)-dehydroabietylamine, n-(4-amino-2-methylphenyl)-4-chlorophthalimide, 1-pyrenebutyric acid, atropin, 1-phenylalanine(4-i), 4-(2,4-di-tert-amylphenoxy)butylamine, 1-diaminopropionic acid(ivdde), 1-lysine(dde), 1-lysine(2-cl-z)-oh, 1-tyrosine(2,6-cl2-bzl), 4,4′-(9-fluorenylidene)-dianiline, 1-hydroxyproline, 4′-carboxy-benzo-18-crown-6, cholic acid as well as compounds having the following structure: 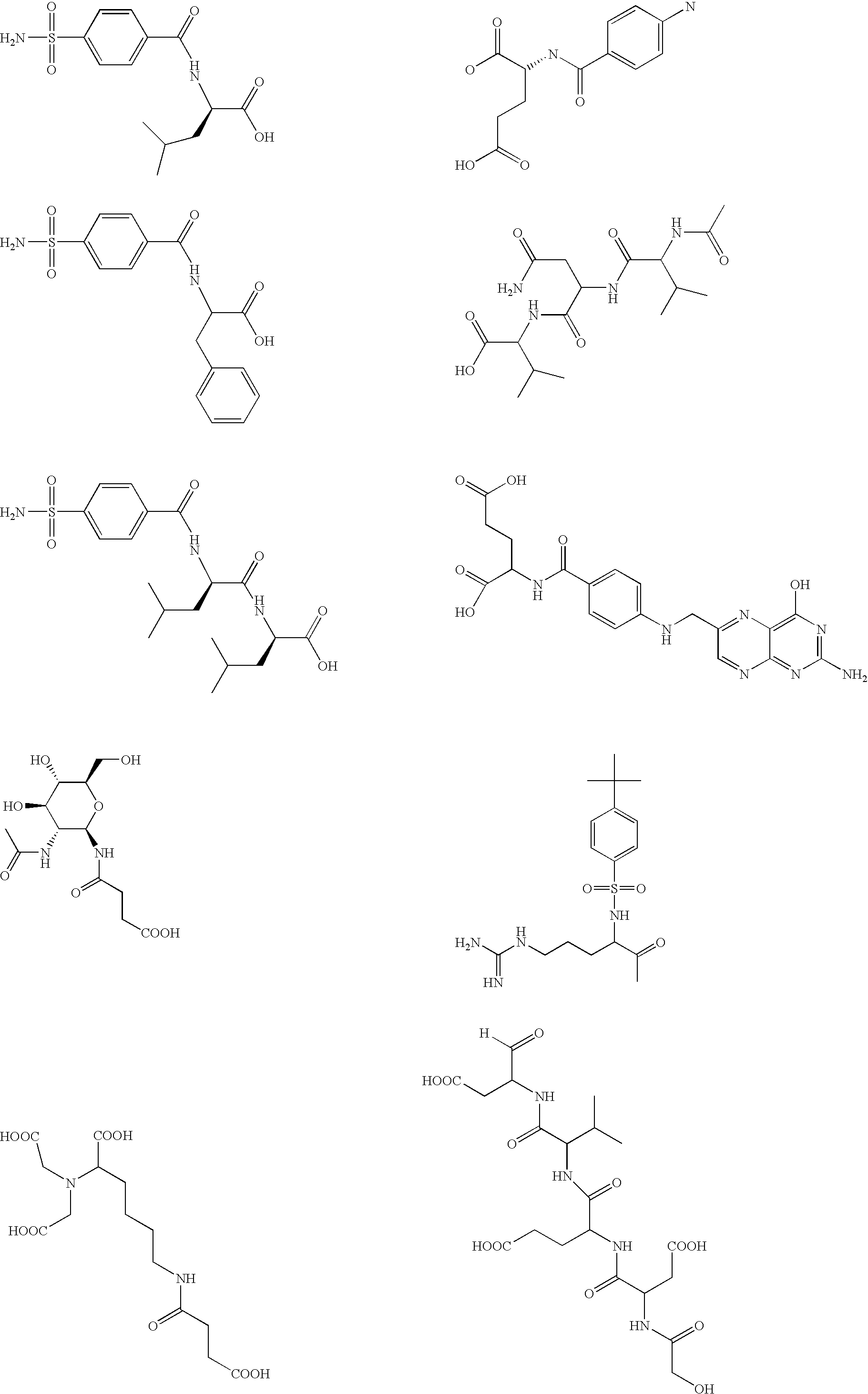 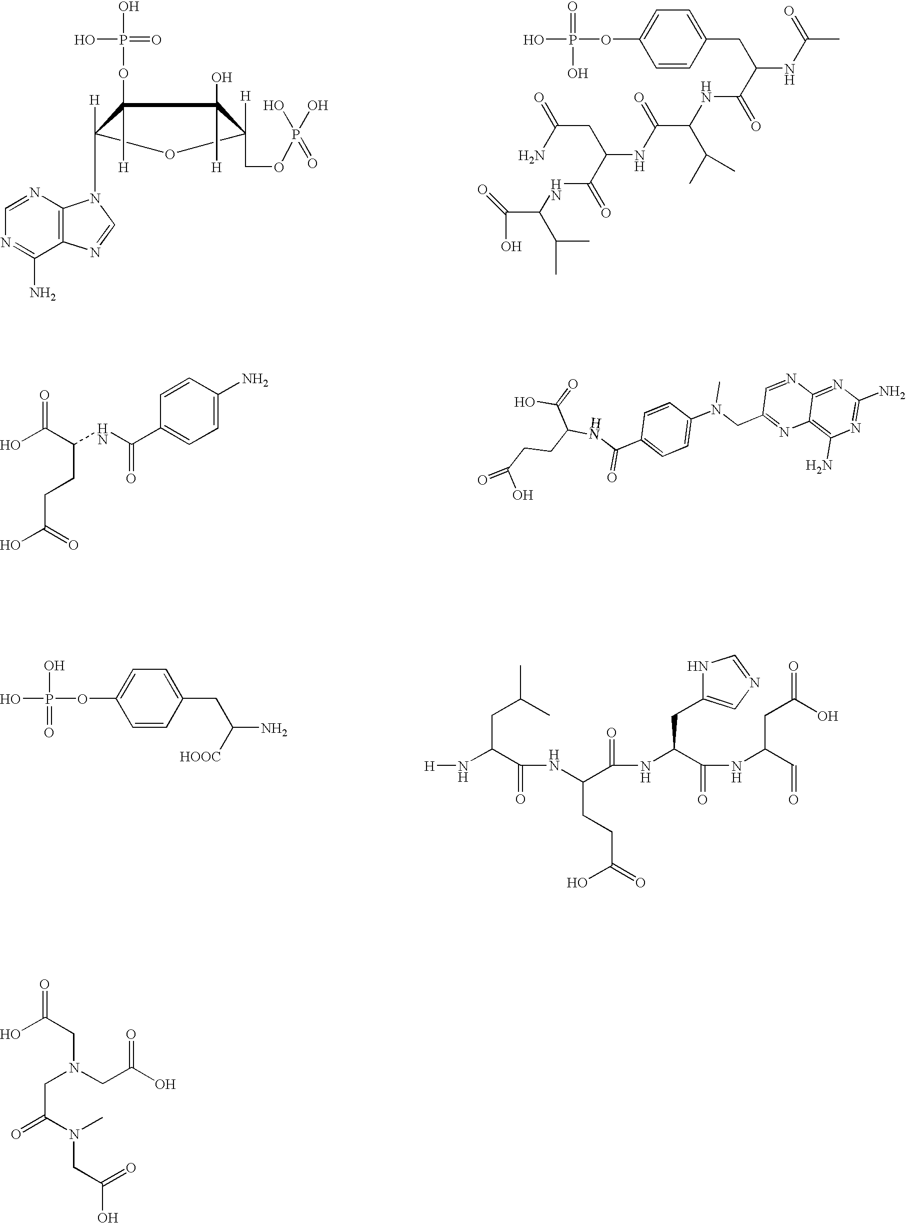 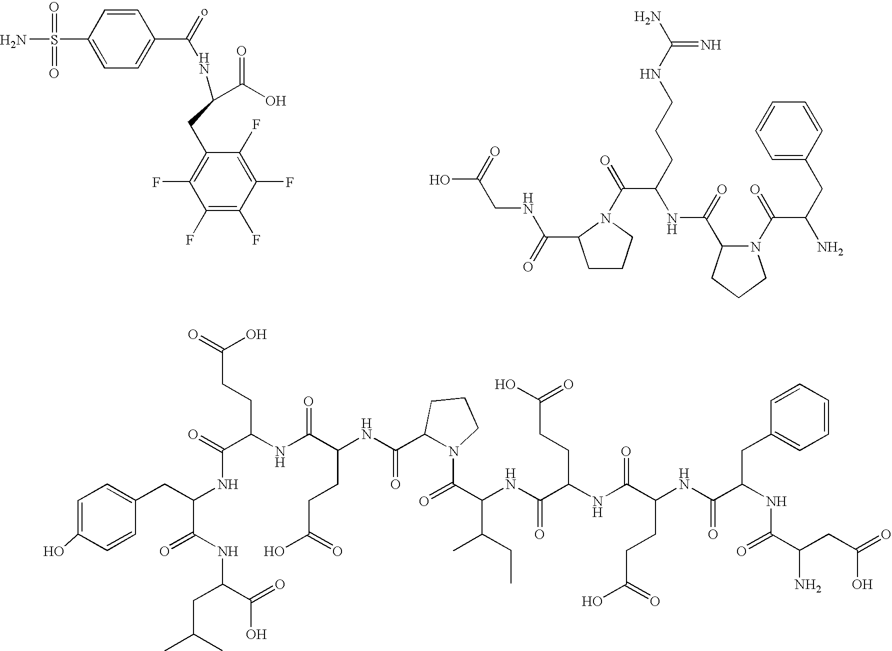
(BZL=benzyl, OBZL=benzyloxy, 2-CL-Z=2-chlorobenzyloxycarbonyl, 2,6-CL2-BZL=2,6-dichlorobenzyloxycarbonyl, DDE=1-(4,4-dimethyl-2,6-dioxocyclohexylidene)ethyl, ivDDE=1-(4,4-dimethyl-2,6-dioxocyclohexylidene)3-methylbutyl.)

This list is neither exhaustive nor limiting and can easily be completed by the person skilled in the art.

If the LAC has an L group, it is represented by a lower molecular compound having a molecular weight of <75, preferably <50 g/mol, such as a C1-C4 alcoxy group, preferably methoxy, or acyloxy, preferably acetyloxy, or amino ethyl. An interaction between the receptor and such a non-ligand L^{N} cannot be excluded; however, the possibility of such an interaction is low on account of the simple structure of such groups. If a two-arm anchor is used which bears both an L and an L^{N} group, the arm ending with the ligand is preferably longer than the functionally unsaturated arm. Particularly good results are obtained at a ratio of approx. 2:1.

The method according to the invention for providing ligand-anchor conjugates comprises the following steps:
a) attachment or synthesis of an anchor to/on a solid phase SP suitable for chemical synthesis;
b) binding or synthesis of a ligand L to/on an anchor generating a ligand-anchor conjugate on the solid phase SP;
c) cleavage of the ligand-anchor conjugate from the solid phase SP.

After cleavage, the LAC may be immobilized on a suitable substrate so as to obtain a sensor surface which is ready for use.

According to the method of the invention, a compound, which is as a rule part of the anchor and carries a functional group, is attached by means of this group to a solid phase which is suitable for solid-phase synthesis. For binding the anchor, solid-phase synthesis is based on compounds which comprise for example the following functional groups: acetals, ketals, acylals, acid halides, alcohols, aldehydes, alkenes, halides, alkines, allenes, amides, amidines, aminals, amines, anhydrides, azides, azines, aziridines, azo compounds, boranes, carbamates, carbodiimides, carboxylic acids, carbonic esters, cyanamides, cyanates, diazo compounds, diazonium salts, epoxides, ethers, hydrazides, hydrazines, hydrazones, hydroxamic acids, hydroxamic esters, hydroxylamines, imides, imines, inorganic esters, isocyanates, isocyanides, isothiocyanates, ketenes, ketones, nitriles, nitro compounds, nitroso compounds, oximes, phenols, phosphines, phosphonates, ammonium salts, phosphonium salts, sulfonamides, sulfones, sulfonic acids, sulfone esters, sulfonium salts, sulfonyl azides, sulfonyl halides, sulfoxides, thioamides, thiocarbamates, thiocyanates, triazenes, ureas or isoureas.

The formation of amide or ester bonds between the solid phase and the anchor is preferred. Here, amino, hydroxyl or carboxyl functions may be present on the solid phase. The anchor or a part of the anchor then comprises a complementary functional group.

During cleavage, these functional groups at the LAC may be rendered in modified or unmodified form (e.g. by reaction with the cleavage reagent, the solvent, by activation, by blockage or in that parts of the linker remain on the anchor). Likewise, these groups or parts of the anchor may remain on the linker and thus on the solid phase.

The solid phase can be a synthetic resin, a synthetic polymer film or a silicon or silicate surface suitable for synthesis. If synthetic resins are used, different types of resins may in principle be used. Particulate, polystyrene- or polystyrene-polyethyleneglycole-based polymeric resins are well established in solid-phase peptide synthesis or combinatorial chemistry and very helpful in carrying out multi-step reaction sequences. Commercially available resins which are used directly or after their modification, preferably by a chemical reaction with glycolic acid, may be used as synthetic resins. For achieving attachment of the anchor, commercially available resins are provided with so-called linkers, i.e. compounds which provide at least two functional groups and are connected both to the solid phase and the anchor during sythesis. In particular, halomethyl resins may be used, such as Merrifield resin. If such resins are used, the functional group on the starting molecule for the LAC synthesis is preferably —COOH or —OH. In case of a carboxyl group (—COOH), the latter is often present as —COOH, —CH₂OH or —COOCH₃ after being cleaved from the resin. In case of a hydroxyl group (—OH), the latter is preferably unmodified after cleavage. In case of an amino group (—NH₂), —NHSO₂— is preferably present. A further advantageous embodiment is based on the use of hydroxy resins, wherein a cyano, carboxyl or hydroxyl group may be present in the aforementioned starting molecule. In case of an isocyano group (—NCO), the cleavage preferably results in a urea derivative —NHCONH—. In case of the carboxyl group, cleavage from the resin preferably results in the following functional groups: —COOH, —CON—, —CH₂OH, —COOCH₃, —CONH₂, —CONH—, —CONHNH₂. Optional free valencies of the above groups are preferably saturated with a C₁-C₄ alkyl group. In case of a hydroxy group, a hydroxy function is preferably formed. In a preferred embodiment, the resin NovaSyne TGA (Calbiochem-Novabiochem AG, Switzerland) is preferably used as solid phase. The use of amino resins has also turned out to be advantageous, a carboxyl function being preferred for bonding which may be present after LAC cleavage from the solid phase as —CONH—, —CHO or —CO—. In a particularly preferred embodiment, Tentagel RAM® (Rapp Polymere, Tübingen) is used as amino resin.

In a further advantageous embodiment, trityl resins are used as solid phase, which may be bound by means of-COOH, —NH₂, —OH, —CONHNH₂. The functional group formed after cleavage from the solid phase is in this case subsequently available in its original form. In a preferred embodiment, 2-chlorotrityl chloride resin (Calbiochem-Novabiochem AG, Switzerland) is used. The use of dihydropyran or carboxy resins as solid phase has also turned out to be advantageous. In a preferred embodiment, a hydroxy group is present in the starting molecule of the LAC synthesis, which hydroxy group remains unmodified after cleavage. In a preferred embodiment, the carboxy resin NovaSyn® TG Carboxy (Calbiochem-Novabiochem AG, Switzerland) or the dihydropyran resin DHP HM-Harz (Calbiochem-Novabiochem AG, Switzerland) is used. Further advantageous embodiments comprise arylsiloxy resins, in which the functional group may be a halogen atom. In this case, after LAC cleavage from the solid phase (the resin), a constituent of the linker or the solid phase is bound to the anchor element since after cleavage the function —Ar—H is preferably present instead of the halogen atom; Ar is an aromatic group originating from the solid phase (the arylsiloxy resin).

As starting molecule for the LAC synthesis and for attachment to the solid phase in a preferred embodiment, commercially available compounds may be used, such as lysin, lysinol or 2,3-diaminopropionic acid as well as their derivatives which are available in twice orthogonally protected form. For binding to the solid, all functionalities present can be used. After cleavage, Y is formed, depending on the cleavage condition, preferably as a methylamide, methylcarboxy or methylhydroxy group.

Cleavage of the ligand-anchor conjugate from the solid phase P may also be induced by intramolecular cyclization. If the linker is a dipeptide, such as Lys-Pro, prolin being bound to the C terminal of the resin, a diketopiperazine [anchor 3, “DKP anchor”] is formed after cleavage from the resin, e.g. by cleavage of a protective group, preferably alpha-tert. butyloxycarbonyl, which induces a spontaneous cyclization. A pyrazolone may also be formed after cleavage from P, if the linker comprises a β-ketocarboxylic acid and phenyl hydrazine is for example used for cleavage. The principle of cleavage from the resin by spontaneous cyclization after deblocking is not restricted to diketopiperazines or pyrazolones.

The list of resins and functions is not exhaustive and can easily be completed by the skilled person. An overview is given in “Novabiochem® Combinatorial Chemistry Catalog & Solid Phase Organic Chemistry Handbook” March 1998, Calbiochem-Novobiochem AG, Switzerland.

With the aforementioned method, combinatorial chemistry may be used for solid-phase synthesis of ligand-anchor conjugates. This entails various advantageous effects, such as the possibility of producing a great number of different conjugates and using them later separately or in combination for drug screening or binding studies.

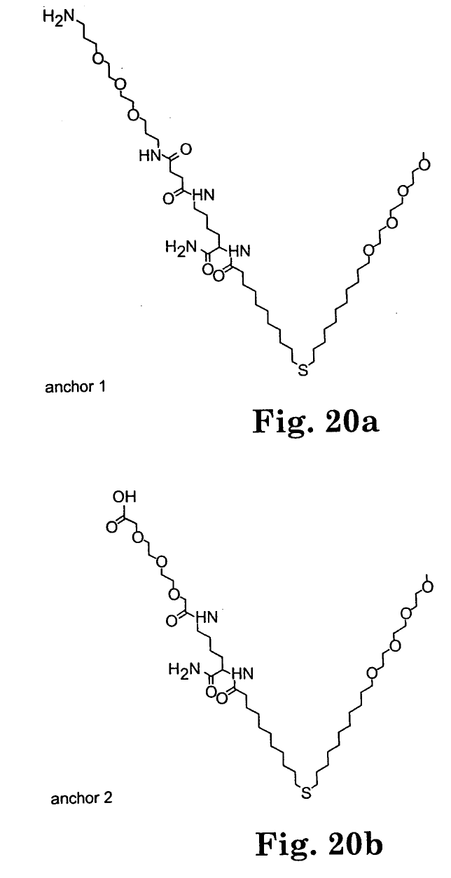

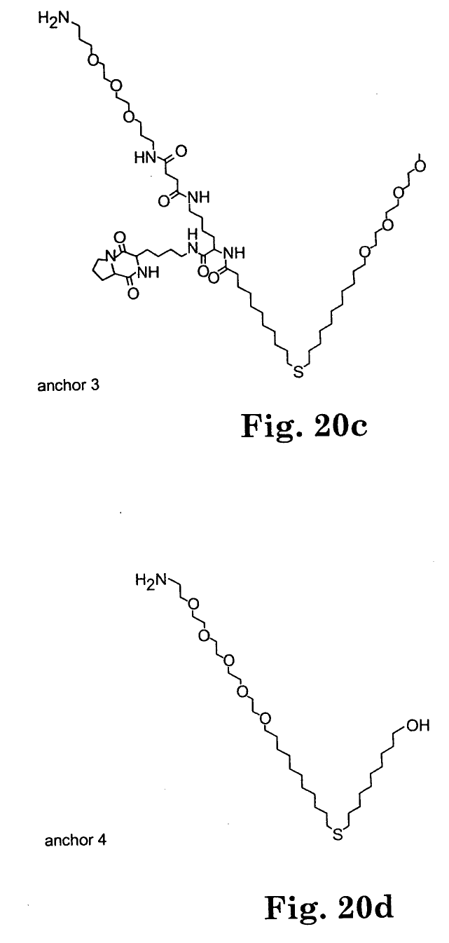

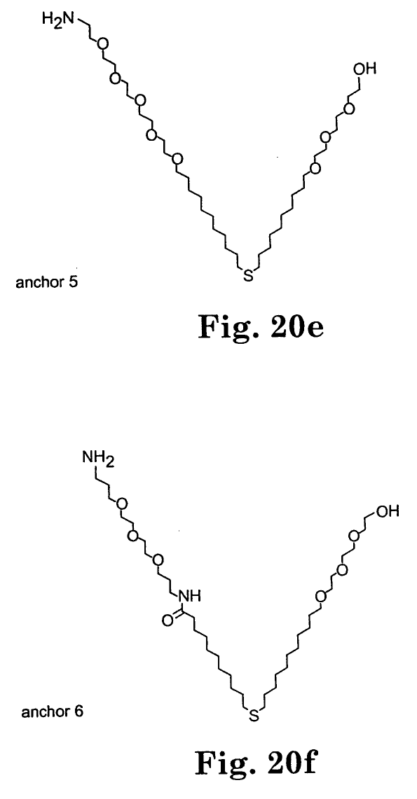

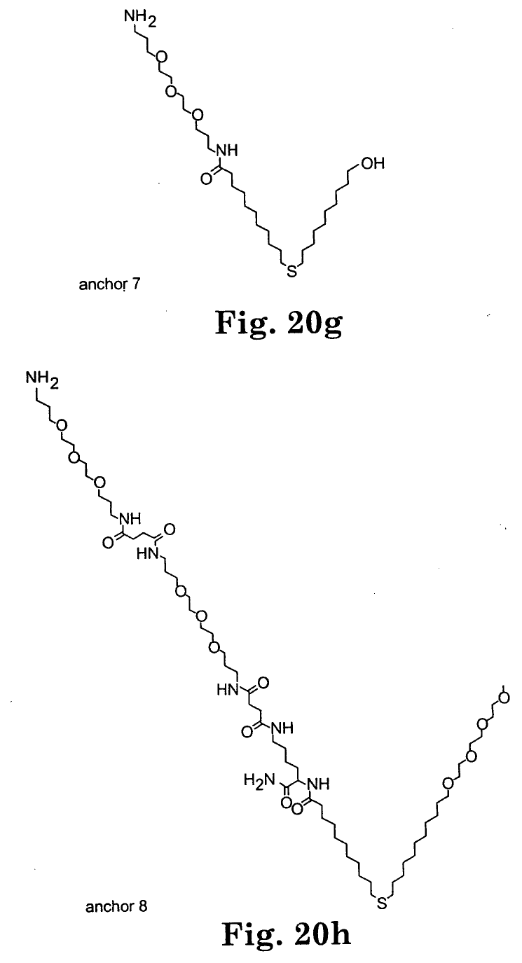

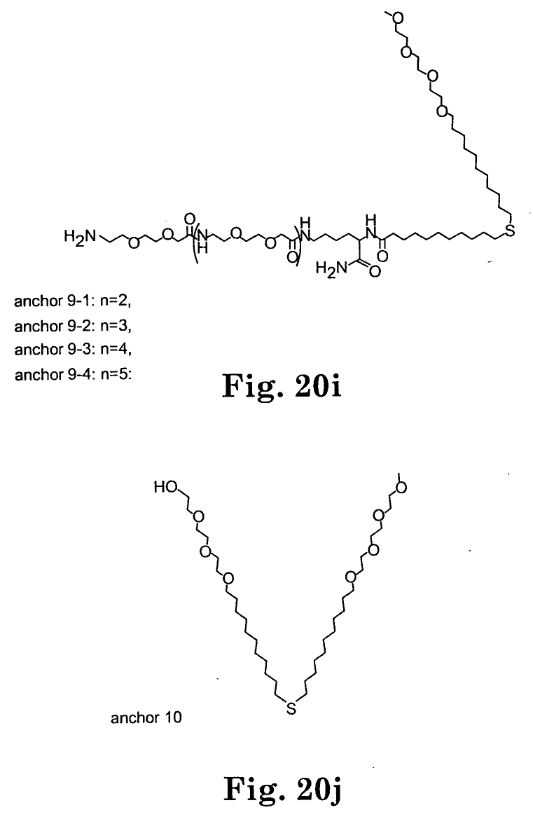

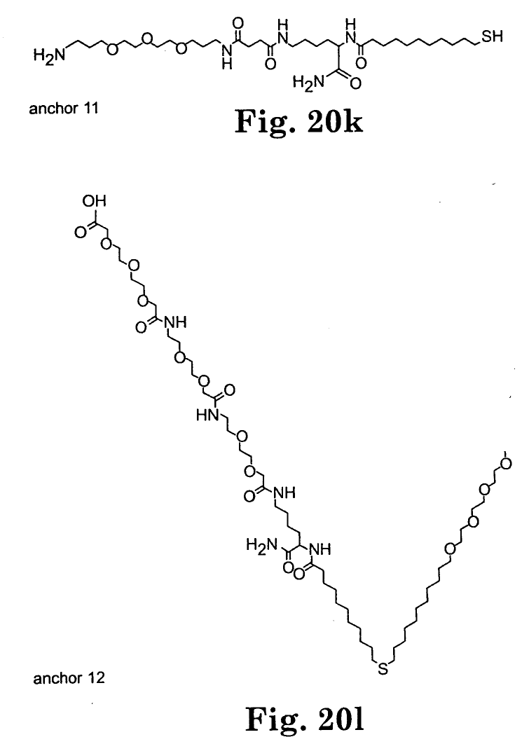

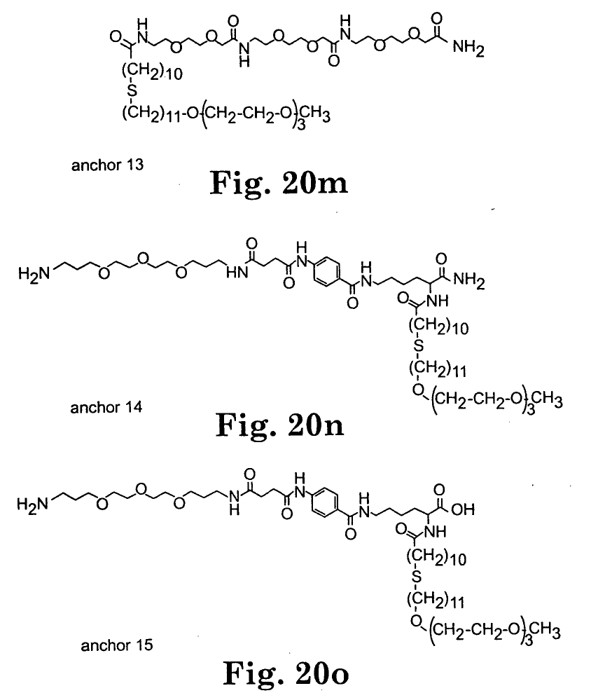

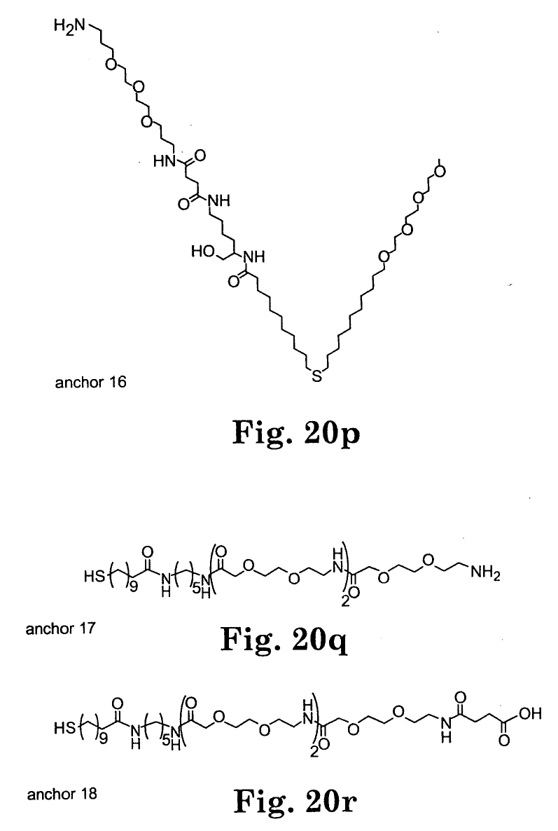

Particularly preferred are combinations of R¹, R^{1a}, R², R^{2a}, X and Y, as evident from the anchor structures 1-3,8-10, 12 and 14-16 in FIG. 20.

A further preferred structure of ligand-anchor conjugates according to FIG. 1 is obtained if, during the synthesis of a two-arm LAC, attachment to the solid phase is carried out at a site which is intended for the ligand or non-ligand. During the LAC cleavage under suitable conditions, the solid phase is subsequently directly replaced by a non-ligand. In this case, the anchor is not synthesized in a convergent but in a linear synthesis (“straight forward”). The above statements as to R¹, R^{1a}, R², R^{2a}, X and Y also apply to the linear anchor synthesis. In this case, combinations of R¹, R^{1a}, R², R^{2a}, X and Y, as can be found in the anchor structures 4-7 (FIG. 20) in the Examples, are particularly preferred.

In connection with the solid-phase synthesis of “diluting components”, i.e. anchors exclusively carrying non-ligands, attachment of the anchor may also be effected to the solid phase at the position of a non-ligand. Thus, this is also a form of linear synthesis. As regards the anchor synthesis, the above statements as to R¹, R^{1a}, R², R^{2a}, X and Y also apply. In this case, combinations of R¹, R^{1a}, R², R^{2a}, X and Y, as can be found in the anchor structures 10 and 13 (FIG. 20), are particularly preferred.

With the method according to the invention, thiol anchors may also be prepared among the structures according to FIG. 1. As regards the anchor synthesis, the above statements as to R¹, R², X and Y are also applicable. In this case, combinations of R¹, R², X and Y, as can be found in anchor structure 11 (FIG. 20), are particularly preferred. Anchors 17 and 18 in this Figure illustrate preferred structures in case Y═H.

The synthesis of a diluent which is not prepared according to the method of the invention is illustrated in Example 2.

If the aforementioned SAM-forming anchors which are provided with a suitable X group are used, it suffices to contact the LACs after cleavage from the solid phase with the substrate to obtain a sensor surface that is ready for use. The substrate may e.g. be incubated in an aqueous LAC solution, or such solutions may be applied to limited portions of carrier surfaces, e.g. by plotting methods. Thus, the parallel use of LACs bearing different ligands is possible. Mixtures of different LACs may alternatively be used.

For generating defined areas on the sensing surface of a sensor enabling the bonding of a receptor, while simultaneously leaving areas of the surface non-active for detection, additional anchors may be applied which do not carry ligands and are exclusively saturated by L^{N} groups. Such so-called “diluting components” may also be used for three-dimensionally isolating the ligands on the surface in order to avoid a passive coverage of immobilized interaction partners if a sterically large receptor is present. As shown in connection with thiols, the ligand density (LAC density) plays an important role in the molecular detection of receptors (B. T. Houseman, M. Mrksich, Angew. Chem. 111 (1999) 876-880). Almost the same applies to sulfides. In order to guarantee an optimum interaction between ligand and receptor, appropriate mixtures of LAC and diluent must be produced and presented on the carrier surface. This may only be reliably achieved if they have been mixed before. This represents a further advantage of the method according to the invention since an synthesis of LAC on the carrier surface cannot guarantee homogeneous dilutions.

In a preferred embodiment, the sensor comprises a carrier plate which exhibits a multitude of regularly arranged, position-addressable fields for immobilizing LAC. If various ligands are combined to form molecular libraries for interaction analysis purposes, LAC of at least one type of ligand can be allocated to each field of the carrier plate. By means of such a measuring arrangement on which different ligands have been immobilized in a well-defined way it becomes possible to present to the analyte a multitude of different ligands in the form of an array. Thus, it is possible to simultaneously subject a large number of (different) biomolecules or receptors to a detection of their biospecific binding properties. Such a parallelization goes hand in hand with the simultaneous minimization of the test set-up and the automation of the analytic process.

In case planar carriers are used, there will be no barrier confining the liquid between the fields. In this case, the liquid droplets or films containing the LAC applied to the gold fields e.g. by means of conventional microplotting methods should be dimensioned in such a way as to prevent the liquid from spilling over. This has to be taken into account even if the carrier has been structured in advance in that, e.g., a gold layer is deposited by means of a sputter technique or by vapor deposition and said layer is subdivided into individual segments by means of photolithography and etching techniques.

U.S. Pat. No. 5,670,322 describes an apparatus in which small compartments which may, e.g., be gold-coated, are produced by means of conventional photolithographic etching techniques. Apparatuses of this type or surface-coated microtiter plates (consisting of PP or PS) on the one hand exhibit the desired liquid barriers, but on the other hand have vertical side walls which are not completely covered with gold when they are coated by means of sputter or vapor deposition techniques. These uncoated spots can then easily be covered unspecifically by analytes (receptors), e.g. proteins/biomolecules. This, however, should be avoided as far as possible when detecting the biospecific binding properties in order to obtain a favorable signal-noise ratio.

Thus, if structured carrier plates are used as sensor surfaces in the present invention, they preferably exhibit a multitude of regularly arranged, position-addressable fields for immobilizing LAC, said fields being localized within cavities of small depth. This provides a liquid barrier while simultaneously keeping the surface as small as possible. Moreover, said fields comprise a layer of the material which enables the immobilisation of the LAC. Preferably, the cavities are of a depth of from 20 to 100 μm and the LAC are immobilized on their bottom which in this case is, e.g., made of a metal or metal oxide, preferably by a noble metal such as gold.

By means of fields of this kind it is possible to avoid or minimize disadvantages as regards unspecific binding as well as spilling over which occurred in the methods which have so far been used. Moreover, such a carrier plate can be prepared at low cost due to the fact that methods and materials used in photolithography and etching techniques as applied in semi-conductor technology are used.

Preferred embodiments of such a carrier plate will be explained in detail in the following with reference to the Figures.

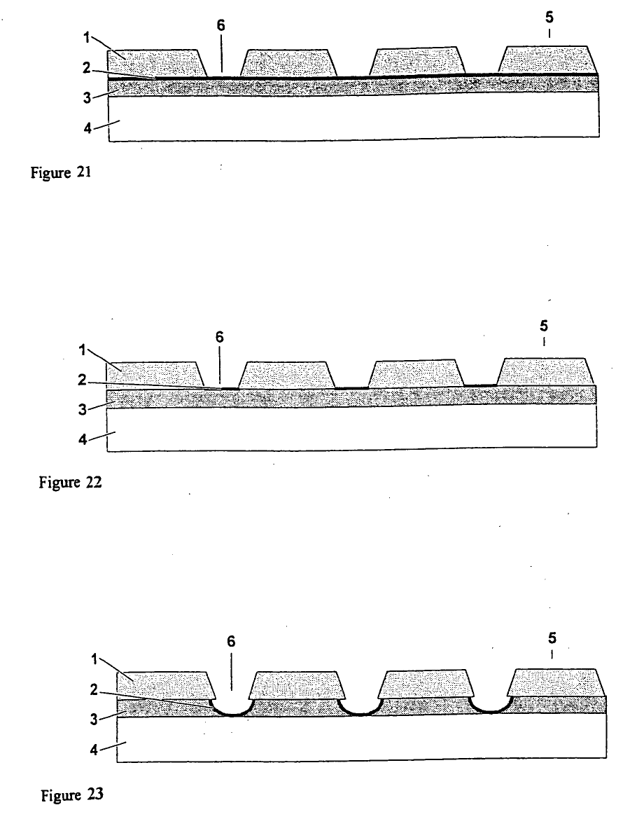

FIG. 21 to 23, respectively, show a schematic section of preferrred carrier plates in cross-section.

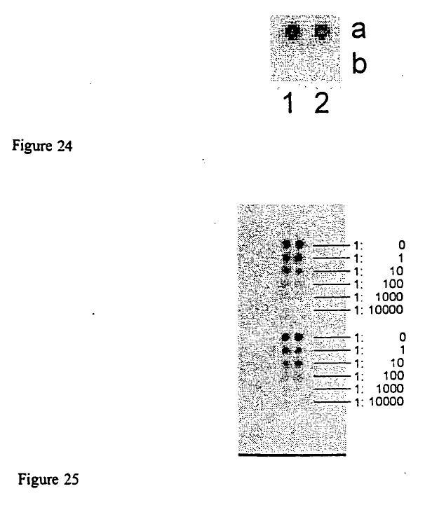

FIG. 24 to 26, respectively, show CCD pictures of luminescence-labelled receptors which have interacted with ligands immobilized on carrier plates.

The preparation of a carrier plate (5) according to FIG. 21 can be started from a copper-clad base material (4) which preferably already has a metal layer (3) such as copper thereon and which is provided with said carrier layer (2) in a galvanic deposition process. The thickness of the carrier layer is a few micrometers only, which exactly corresponds to the thickness required to prepare a continuous coating. Subsequent to the galvanic process the plate is provided with a protective layer (1) which can be exposed to UV. Either photoresists commonly used in semi-conductor production or other protective lacquers which can be exposed to UV light and, thus, can be structured may be used for this purpose.

The lacquer layers used preferably have a thickness of from 20 μm to 100 μm. In an exposure step, an image of a mask is projected onto the protective layer. The mask preferably exhibits round or rectangular/square patterns. Subsequent to a developing step, defined openings will form in the protective layer which expose the carrier layer underneath. Thus, after having been structured, the photo-structurable protective layer will simultaneously form the walls of the cavities (6) and, thus, determine the shape of the cavity (6) and its opening.

If the protective layer (1) is applied and structured prior to the application of the carrier layer (2), a carrier (5) according to FIG. 22 will be obtained.

FIG. 23 illustrates a carrier plate (5) having deeper cavities (6), the proportion of unprotected wall surface not being increased, however. This carrier plate preferably exhibits a base material (4) having a metallic coating (3) provided on the surface thereof which coating in turn is provided with a protective layer (1), at least one cavity (6) being formed in said protective layer (1) and in said metallic layer (3) which is trough-shaped in the area of the metallic layer (3) and is provided with a carrier layer (2) and which, in the area of the protective layer (1) is tapered towards the trough-shaped part, the lower edge of the section of the cavity provided in the protective layer (1) being of a smaller diameter than the upper edge of the section of the cavity formed in the metallic layer (3).

The preparation of a carrier plate as illustrated in FIG. 23 also starts from a coated plate. In this case, however, the thickness of the layer (3) which is already present is preferably from 100 μm to 150 μm. The layer (3) is structured by means of a photoresist (not shown in the Figure) in such a way that it already exhibits recesses. Subsequently, the carrier layer (2) is galvanically deposited on this plate. In a second photolithographic step a protective layer (1) is then also structured in such a way that a structure is formed in the protective layer (1) on top of the cavities (6) etched into layer (3). In this case the depth of the cavity (6) formed is determined by the depth of the etched structure together with the thickness of the protective layer (1).

The cavities are preferably arranged in such a way that a regular, preferably Cartesian grid of columns and lines is produced on the carrier plate. The size and the shape of the carrier plate can be chosen arbitrarily and can easily be adapted to the detection system used. If drop spot robots are used to immobilise the LAC or if the LAC are present on microtiter plates, the distance of the fields from one another will preferably have to be adapted to the microtiter format or drop spot device used, respectively. The number of fields kann also exceed the number of subunits of the microtiter plate, i.e. multiple fields may be used per area. Thus, a square carrier plate having a lateral length of about 12 cm may, e.g., have 9216 fields altogether which may be covered using a pipetting robot from six conventional 1536 microtiter plates.

Another advantage of such a carrier plate is that it can be separated into segments by sawing, cutting or punching.

However, a structured presentation of the same or different LAC can also be achieved by immobilizing the LAC on a spatially separate section of the sensor surface after the selective application of a liquid volume without requiring the physical separation of a carrier into individual compartments. Individual fields containing LAC can also be produced on the surface by selectively applying solutions of LAC, e.g. by means of pipetting methods, drop spot methods, stamping methods or ink jet methods. Techniques described in EP-A-0 872 735 for applying reagent spots onto metallic or metal oxide surfaces can preferably be used analogously.

If the sensor surface or the carrier plate serve to present a molecular library, different types of LAC are preferably immobilized which differ from field to field. Within one field the same LAC as well as mixtures of different LAC can be used. A carrier plate having the above dimensions can, thus, present up to 9216 different ligands or mixtures of ligands to the analyte.

The sensor surface according to the invention is preferably used for electrochemical and spectroscopic measurements of molecular interactions between immobilized ligands and non-immobilized interaction partners, in particular biomolecules. It can thus advantageously be used in medical diagnostics.

On account of their aforementioned advantages, the surfaces according to the invention may, however, also be used in conventional methods of chromatography and purification, such as in affinity chromatography.

Molecules acting as receptors are molecules which are preferably present in biological systems or interact with the latter, such as proteins, DNA, RNA, oligonucleotides, prosthetic groups, vitamins, lipids, mono-, oligo- and polysaccharides, but also synthetic molecules, such as fusion proteins and synthesized primers.

For detecting a receptor binding to the sensor surface, known mass-sensitive and/or optical methods are available. Optical methods, such as for example SPR spectroscopy or chemoluminescence measurements, are preferred.

### SYNTHESIS EXAMPLES

### Example 1

### 24,27,30,33-Tetraoxa-12-thia-tetratriacontanoic Acid

### 1.1 (rac)-Tetrahydro-2-pyranyl-(11-bromo-1-undecyl)ether

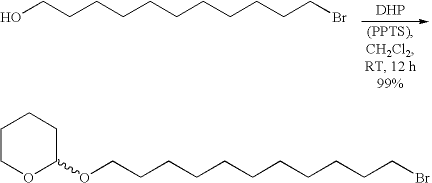

25.1 g (100 mmol) 11-bromo-1-undecanol, 12.6 g (150 mmol) dihydropyran and 2.5 g (10 mmol) pyridinium-p-toluene sulfonate were stirred in 700 ml dichloromethane for 12 hrs at room temperature. Then the mixture was diluted with diethylether and extracted with semiconcentrated sodium chloride solution. After drying over sodium sulfate and removing the solvent, 33.2 g (99 mmol, 99%) TLC-pure product were obtained as yellowish oil.

R_{f}=0.42 (silica gel, c-hexane/ethyl acetate=9:1)

¹H-NMR (500 MHz, CDCl₃, 303 K): δ=4.56 (t, 1H), 3.86 (dt, 1H), 3.72 (dt, 1H), 3.47-3.51 (m, 1H), 3.36-3.40 (m, 3H), 1.79-1.88 (m, 3H), 1.68-1.73 (m, 1H), 1.38-1.64 (m, 6H), 1.39-1.44 (m, 2H), 1.23-1.37 (m, 12H).

### 1.2 (rac)-Tetrahydro-2-pyranyl-(12,15,18,21-tetraoxa-1-docosyl)ether

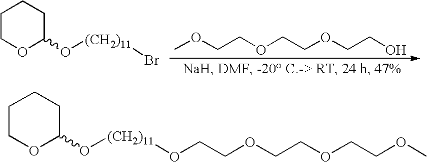

A solution of 12.6 g (76.5 mmol) triethylene glycol monomethyl ether in 50 ml N,N-dimethyl formamide was added dropwise to a suspension of 1.84 g (76.5 mmol) sodium hydride in 150 ml N,N-dimethyl formamide (cooled to −20° C.) under an argon atmosphere. After stirring the mixture for 15 min at −20° C. a solution of 25.2 g (75.0 mmol) (rac)-tetrahydro-2-pyranyl-(11-bromo-1-undecyl)ether in 50 ml N,N-dimethyl formamide was added dropwise within 45 min. The reaction was stirred in a Dewar flask overnight without further cooling and warmed up to room temperature. The solvent was then removed on a rotary evaporator and the residue was dissolved in 500 ml dichloromethane. Insoluble salts were filtered off and the solution was extracted three times with 150 ml water each. After drying over sodium sulfate and removing the solvent on 400 g silica gel with c-hexane/ethyl acetate (4:1→2:1) the solution was subjected to chromatography. 14.7 g (35.0 mmol, 47%) TLC-pure product were isolated as yellowish oil.

R_{f}=0.37 (silica gel, c-hexane/ethyl acetate=1:1)

¹H-NMR (500 MHz, CTLCl₃, 303 K): δ=4.53 (t, 1H), 3.86 (dt, 1H), 3.68 (dt, 1H), 3.59-3.64 (m, 8H), 3.50-3.55 (m, 4H), 3.44-3.48 (m, 1H), 3.41 (t, 2H), 3.35 (dd, 1H), 1.76-1.85 (m, 1H), 1.63-1.70 (m, 1H), 1.45-1.58 (m, 8H), 1.22-1.34 (m, 14H).

### 1.3 12,15,18,21-Tetraoxa-1-docosanole

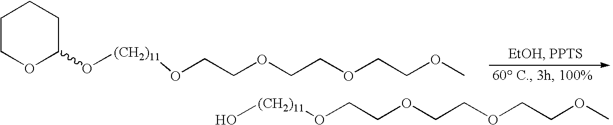

14.7 g (35.0 mmol) (rac)-tetrahydro-2-pyranyl-(112,15,18,21-tetraoxa-1-docosyl)ether were dissolved in 300 ml ethanol, mixed with 1 g (4 mmol) pyridinium-p-toluene sulfonate and stirred at 60° C. for 3 hrs. When the reaction was completed (TLC control), the solvent was removed on a rotary evaporator. The residue was dissolved in 300 ml diethylether, the catalyst that did not dissolve was filtered off and the solvent was removed on a rotary evaporator. The product was obtained in quantitative yields as colorless oil.

R_{f}=0.14 (silica gel, c-hexane/ethyl acetate=1:1)

¹H-NMR (500 MHz, CDCl₃, 303 K): δ=3.55-3.60 (m, 8H), 3.53 (t, 2H), 3.46-3.50 (m, 4H), 3.38 (t, 2H), 3.23 (s, 3H), 2.19 (bs, 1H), 1.44-1.52 (m, 4H), 1.19-1.29 (m, 14H).

### 1.4 12,15,18,21-Tetraoxa-1-docosyl-p-toluenesulfonic Ester

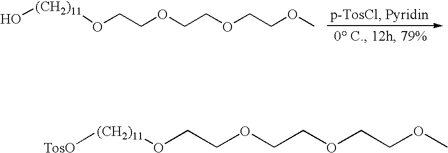

11.7 g (35.0 mmol) 12,15,18,21-tetraoxa-1-docosanole were dissolved in 150 ml pyridine and cooled to 0° C. To the mixture, 14.3 g (75.0 mmol)ₚ-toluene sulfonylchloride were slowly added. The reaction was left to stand overnight at 4° C. Pyridinium chloride was precipitated in the form of long needles. The completion of the reaction was determined via TLC control. The entire mixture was poured under stirring onto 500 g ice and then repeatedly extracted with diethylether. The combined organic phases were washed three times with 1 M hydrochloric acid and three times with cold water. After drying over potassium carbonate/sodium sulfate the solvent was concentrated to about 50 ml and this solution was filtrated with 100 g silica gel using dichloromethane as eluent. After removal of the solvent 13.5 g (27.6 mmol, 79%) product were obtained as colorless oil.

R_{f}=0.39 (silica gel, c-hexane/ethyl acetate=1:1)

¹H-NMR (500 MHz, CDCl₃, 303 K): δ=7.77 (d, 2H), 7.32 (d, 2H), 4.00 (t, 2H), 3.51-3.68 (m, 12H), 3.42 (t, 2H), 3.36 (s, 3H), 2.42 (s, 3H), 1.59-1.65 (m, 2H), 1.51-1.59 (m, 2H), 1.19-1.31 (m, 14H).

### 1.5 24,27,30,33-Tetraoxa-12-thia-tetratriacontanoic acid

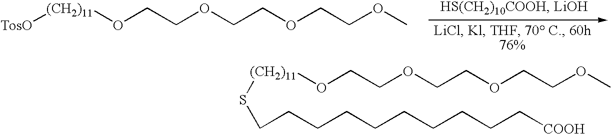

10.7 g (450 mmol) lithium hydroxide and 18.9 g (450 mmol) lithium chloride were suspended in 450 ml tetrahydrofuran and stirred 15 min at room temperature. To the suspension, 17.6 g (80.6 mmol) 11-mercaptoundecanoic acid were first added and after 15 min stirring 18.0 g (110 mmol) potassium iodide. To this mixture a solution of 10.0 g (20.5 mmol) 12,15,18,21-tetraoxa-1-docosyl-p-toluene sulfonic acid ester in 50 ml tetrahydrofuran were added. The reaction was heated under TLC control under reflux until the reaction was completed (about 60 hrs). After cooling the mixture down to room temperature it was acidified with about 40 ml 32% hydrochloric acid to pH=2. Then the solvent was removed on a rotary evaporator, the residue was dissolved in dichloromethane and salts that were not dissolved were filtered off. The crude product obtained was applied on 50 g silica gel and subjected to chromatography with c-hexane/ethyl acetate (1:1) on 500 g silica gel. The product obtained was again recrystallized from n-pentane. 9.09 g (15.5 mmol, 76%) of a white, finely crystalline powder were obtained.

R_{f}=0.26 (silica gel, c-hexane/ethyl acetate=2:3)

¹H-NMR (500 MHz, CDCl₃, 303 K): δ=3.63-3.67 (m, 8H), 3.54-3.59 (m, 4H), 3.45 (t, 2H), 3.38 (s, 3H), 2.50 (t, 4H), 2.34 (t, 2H), 1.55-1.67 (m, 8H), 1.26-1.41 (m, 26H).

### Example 2

### Bis-(12,15,18,21-tetraoxa-1-docosyl)sulfide

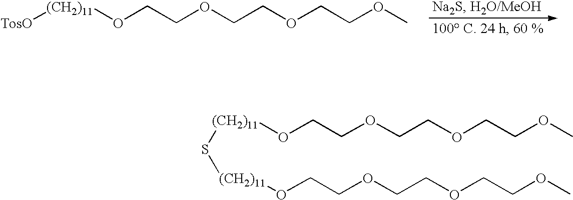

2.8 g (5.0 mmol) 12,15,18,21-tetraoxa-1-docosyl-p-toluene sulfonic acid ester and 680 mg (about 2.80 mmol) sodium sulfide hydrate were heated in a mixture of 40 ml water and 20 ml methanol for 24 hrs under reflux. When the reaction was completed (TLC control) and cooled to room temperature, the solution was extracted with dichloromethane and the combined organic phases were dried over sodium sulfate and the solvent was removed on a rotary evaporator. For purification of the mixture, the crude product was applied on 15 g silica gel and subjected to chromatography on 180 g silica gel with c-hexane/ethyl acetate (2:3) as eluent. The product obtained was again recrystallized from n-pentane at −20° C., whereby 950 mg (1.48 mmol, 60%) purely white, crystalline product were obtained.

R_{f}=0.21 (silica gel, c-hexane/ethyl acetate=2:3)

¹H-NMR (500 MHz, CDCl₃, 303 K): δ=3.62-3.66 (m, 8H), 3.54-3.58 (m, 4H), 3.44 (t, 2H), 3.38 (s, 3H), 2.49 (t, 2H), 1.53-1.60 (m, 4H), 1.26-1.40 (m, 14H).

### Example 3

### Synthesis of Ligand-Anchor Conjugates (LACs) Based on Anchor1

The synthesis of LACs based on anchor1 was carried out in a polypropylene syringe with a polypropylene frit on TentaGel-RAM® resin.

Standard cycle for the coupling of N-Fmoc protected amino acids and of 24, 27, 30, 33-tetraoxa-12-thia-tetratriacontanoic acid of Example 1:

For the cleavage of the Fmoc group 500 mg (0,24 mmol/g) TentaGel-RAM® resin were shaken for 20 min with 5 ml 20% piperidine in DMF. Then the resin was washed five times with DMF. 5 equivalents of the Fmoc amino acid (0.48 mmol) and 5 equivalents (75 mg, 0.48 mmol) 1-hydroxy-1H-benzotriazole (HOBt) were dissolved in 1.5 ml DMF and mixed with 5 equivalents (72 μl, 0.48 mmol) N,N′-diisopropylcarbodiimide (DIC). This solution was added to the resin and the suspension was shaken for 60 min. Then the resin was washed five times with DMF.

Synthesis of Anchor 1 (See FIG. 5):

1a, 1b) Coupling of Fmoc-Lys(Dde)-OH was carried out according to the standard cycle. 2a) Coupling of 24,27,30,33-tetraoxa-12-thia-tetratriacontanoic acid was carried out according to the standard cycle with a coupling time of 6 hrs.

2b) For cleavage of the Dde protecting group, the resin was incubated four times for about 3 min each with 2.5% hydrazine in DMF. Then the resin was washed five times with DMF.

3) The coupling of the succinic acid was carried out by incubating the resin with 2/1/17 (w/v/v) succinic anhydride/pyridine/DMF for 60 min. Then the resin was washed five times with DMF.

4) Pentafluorophenyl ester (Pfp ester) was prepared by incubating the resin with a solution of 200 μl (1,16 mmol) trifluoroacetic acid pentafluorophenyl ester and 100 μl (1,24 mmol) pyridine in 500 μl DMF for 2 hrs. Then the resin was washed five times with DMF.

5) The coupling of 1,13-diamine-4,7,10-trioxatridecane was carried out by incubating the resin with a solution of 500 μl 1,13-diamine-4,7,10-trioxatridecane and 50 mg HOBt in 500 μl DMF for 90 min. Then the resin was washed five times with DMF.

6) Coupling of the ligands

### 6a) Acetyl-anchor1

The amine group of anchor1 was acetylated by incubating 50 mg resin with a solution of 50 μl acetic anhydride and 251 μl pyridine in 300 μl DMF. Then the LAC was removed from the resin.

General protocol for the cleavage of the ligand-anchor conjugate from the TentaGel-RAM resin:

After synthesis, the resin (50 mg) was washed five times with DMF and three times with DCM. Then the resin was incubated for 2 hrs with 1 ml 92/4/4 (v/v/v) TFA/triethylsilane/water and shaken from time to time. The solution was removed from the resin and the resin was washed twice with 250 μl TFA. The pooled solutions were introduced together with nitrogen and the residue was purified by RP-HPLC.

Characterization 6a):

ESI-MS (calculated): (M+H)⁺ 1007.1 (1006.7); (M+2H)²⁺ 504.6 (504.2)

### 6b) Acetyl-O-phospho Tyrosyl-anchor1

Fmoc-Tyr(PO₃H₂)—OH was coupled according to the general protocol by adding 2 equivalents ethyl-diisopropylamine. After cleavage of the Fmoc protecting group the free amine group was acetylated according to 6a). LAC was cleaved according to the general protocol.

Characterization:

ESI-MS (calculated): (M+2H)²⁺ 625.6 (625.8)

### 6c) Acetyl-Gly-Arg-Gly-Asp-Ser-Pro-Lys-anchor1

The coupling of the L-amino acids was carried out according to the standard cycle using the following amino acid derivatives: Fmoc-Gly-OH, Fmoc-Lys(Boc)-OH, Fmoc-Ser(tBu)-OH, Fmoc-Asp(OtBu)-OH, Fmoc-Arg(Pbf)-OH. Acetylation was carried out according to 6a. LAC was cleaved according to the general protocol.

Characterization:

ESI-MS (calculated): (M+2H)²⁺ 853.2 (853.1); (M+3H)³⁺ 569.0 (569.1)

### 6d) Acetyl-(D-Phe)-Pro-Arg-Pro-Gly-anchor1

The coupling of the amino acids was carried out according to the standard cycle. Acetylation was carried out according to 6a. LAC was cleaved according to the general protocol.

Characterization:

ESI-MS (calculated): (M+2H)²⁺ 782.3 (781.6); (M+2H+NH₄)³⁺ 527.1 (527.0)

### 6e) γ-Glu-Cys(StBu)-Gly-anchor1

The coupling of the amino acids was carried out according to the standard cycle using the following amino acid derivatives: Fmoc-Glu(OH)-OtBu, Fmoc-Cys(StBu)-OH and Fmoc-Gly-OH. LAC was cleaved from the resin according to the general protocol.

Characterization:

ESI-MS (calculated): (M+2H)²⁺ 672.0 (672.0)

In a derivative of 6e the StBu-protective group of the cystein was removed before cleavage of the LAC from the resin. For this purpose, the resin (50 mg) was shaken with 1 ml 200 mM dithiothreitol in 3/2 (v/v) sodium phosphate buffer, pH 7.5/acetonitril for 2 hrs under a nitrogen atmosphere. Then the resin was washed five times with water and five times with DMF. LAC was cleaved from the resin according to the general protocol.

Characterization:

ESI-MS (calculated): (M+2H)²⁺ 627.8 (627.9)

### 6f) (2-Trimethylammonium ethyl)-succinyl-anchor1 (Succinic Acid Choline Ester Anchor 1)

The coupling of the succinic acid was carried out according to 3). The choline ester was prepared by shaking the resin (50 mg) with a solution of 100 mg (0.4 mmol) (2-bromoethyl)-trimethylammonium bromide, 20 μl ethyl diisopropylamine in 1.2 ml DMSO for 5 hrs at room temperature. LAC was removed from the resin according to the general protocol.

Characterization:

ESI-MS (calculated): M+1149.9 (1149.8); (M+H)²⁺ 575.8 (575.8)

### Example 4

### Synthesis of Ligand-Anchor Conjugates (LACS) Based on Anchor2

The synthesis of the anchor2-based LACs was performed analogously up to reaction step 2b of Example 3 (FIG. 5).

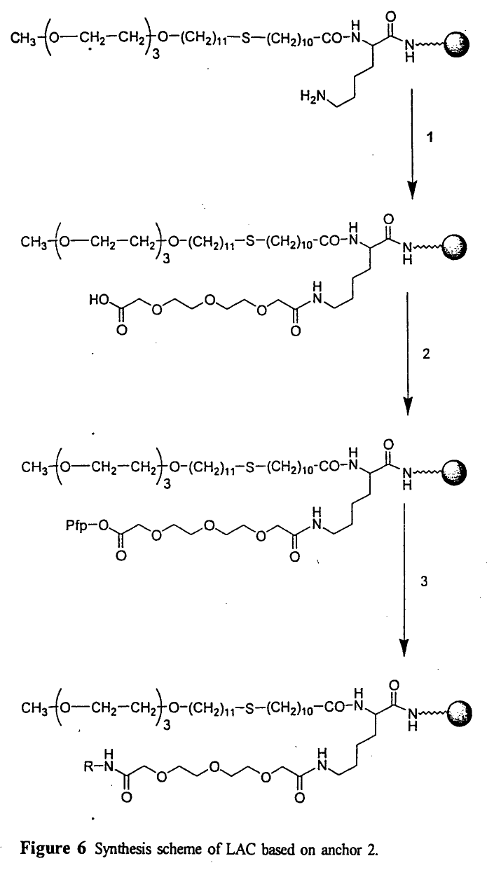

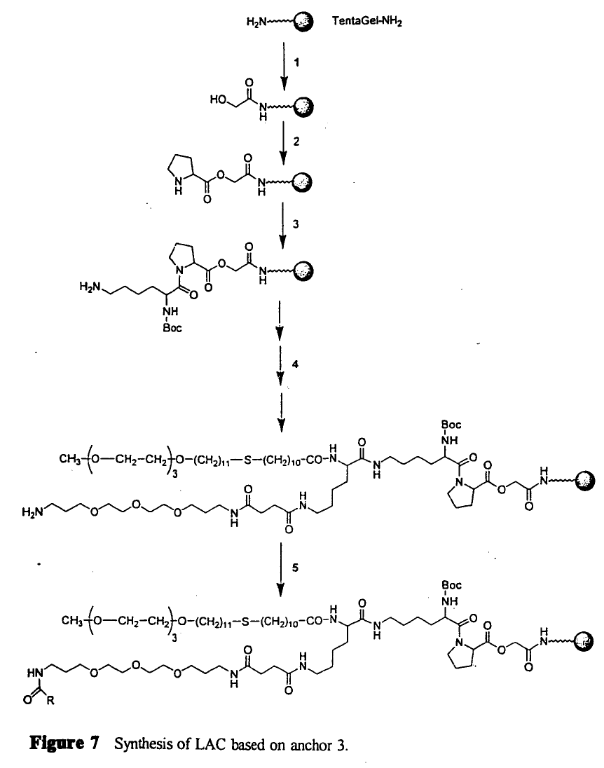

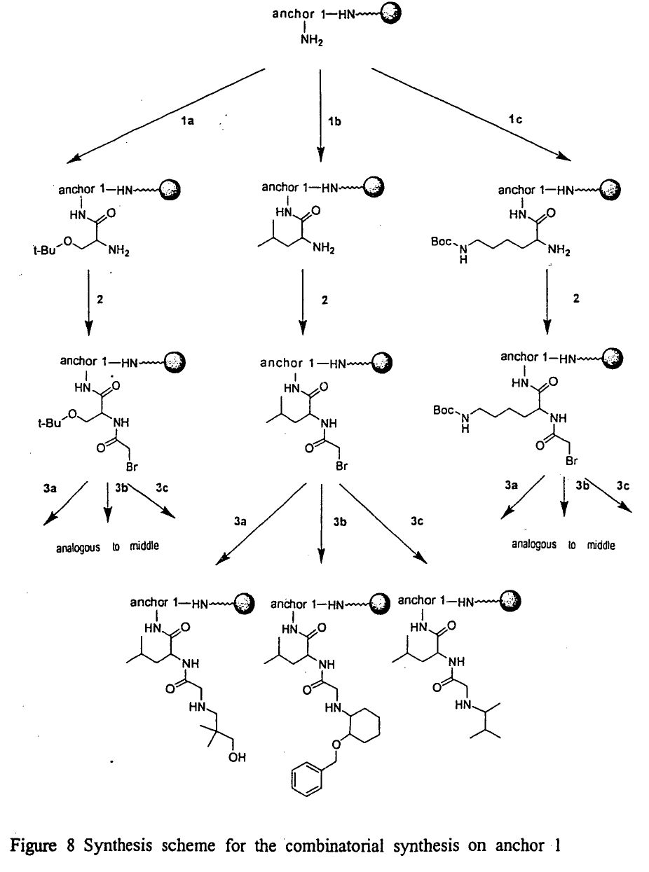

Anchor1. The synthesis scheme is shown in FIG. 6.

1) The coupling of 3,6,9-trioxaundecanediacid was carried out by incubating 500 mg resin with 200 mg (0.9 mmol) trioxaundecanediacid and 134 μl (0.9 mmol) diisopropylcarbodiimide in 2 ml DMF for 2 hrs. Then the resin was washed five times with DMF.

2) The Pfp ester was prepared according to step 4) of Example 3.

3) Coupling of the ligands

### 3a) 2-Acetamido-1-amino-1,2-dideoxy-β-D-glucopyranosyl-anchor2

50 mg resin were shaken for 1 hr with a solution of 20 mg (0.09 mmol) 2-acetamido-1-amino-1,2-dideoxy-β-D-glucopyranose, 14 mg (0.09 mmol) HOBt and 16 μl (0.09 mmol) ethyl-diisopropylamine in 300 μl DMSO. Then the resin was washed three times with DMSO and three times with DMF. LAC was cleaved from the resin according to the general protocol.

Characterization:

ESI-MS (calculated): (M+H)⁺ 1069.0 (1068.7); (M+H+NH₄)²⁺ 543.8 (543.7)

### 3b) Nα,Nα-bis(carboxymethyl)-L-εLys-anchor2

50 mg resin were shaken for 1 hr with a solution of 23 mg (0.06 mmol) Nα,Nα-bis(carboxymethyl)-L-lysine-trifluoroacetic acid salt, 9.36 mg (0.06 mmol) HOBt and 52 μl (0.3 mmol) ethyldiisopropylamine in 500 μl DMSO. Then the resin was washed three times with DMSO and three times with DMF. LAC was cleaved from the resin according to the general protocol.

Characterization:

ESI-MS (calculated): (M+H)⁺ 1110.1 (1110.7); (M+2H)²⁺ 556.2 (556.7)

### 3c) N⁶-aminohexyl-adenine Anchor2

N⁶-aminohexyladenine was prepared according to D. B. Craven et al., Eur. J. Biochem. (1974), 41, 329-333 and purified by RP-HPLC.

The coupling of aminohexyladenine was carried out by incubating the resin (40 mg) with 5 mg (0.014 mmol) aminohexyladenine trifluoroacetic acid salt, 4 mg HOBt and 5 μl DIEA in 200 μl DMF for 2 hrs. Then the resin was washed five times with DMF.

Characterization:

ESI-MS (calculated): (M+2H)²⁺ 542.2 (542.3).

### 3d) N⁶-Aminohexyl-adenosine-5′-monophosphate-anchor2

The synthesis was carried out according to 3c)

Characterization:

ESI-MS (calculated): (M+2H)²⁺ 648.2 (648.3)

### Example 5

### Synthesis of Ligand-Anchor Conjugates (LACs) Based on Anchor3

The synthesis of the LACs based on anchor3 was carried out on TentaGel-NH₂® resin.

1) The coupling of the glycolic acid was carried out by incubating the resin (500 mg, 0.31 mmol/g) with a solution of 80 mg (1.05 mmol) glycolic acid, 164 mg (1.05 mmol) HOBt and 157 μl DIC in 2 ml DMF for 30 min. Then the resin was washed five times with DMF.

2) Esterification of the resin-bound glycolic acid with Fmoc-Pro-OH was carried out by incubating the resin with 210 mg (0.623 mmol) Fmoc-L-Pro-OH, 50 μl (0.63 mmol) N-methylimidazole and 94 μl (0.63 mmol) DIC for 2 hrs. Then the resin was washed five times with DMF, the Fmoc group was removed (see Example 3—standard cycle) and the resin was again washed five times with DMF.

3) The coupling of Boc-L-Lys(Fmoc)-OH was carried out according to the standard cycle.

4) The following steps were carried out according to the synthesis of anchor1, steps 1 to 5.

5a) Acetyl-anchor3

Acetylation was carried out according to step 6a of Example 3.

General protocol for cleavage of the anchor3-based LAC under formation of diketopiperazine:

The resin (50 mg) containing the LACs was washed three times with dichloromethane. For cleavage of the Nα-Boc protecting group the resin was incubated for 30 min with 2 ml 1/1 (v/v) trifluoroacetic acid/dichloromethane. Then the resin was washed five times with dichloromethane and dried in vacuo. Then the resin was washed once with water and then shaken for 12 hrs in 3/2 (v/v) 0.1 M NH₄HCO₃/acetonitril. The solution was removed from the resin and lyophilized. The LAC was dissolved in 1/1 water/acetonitril and acidified with trifluoroacetic acid prior to the HPLC purification.

Characterization:

ESI-MS (calculated): (M+2H)²⁺ 608.4 (608.4)

### 5b) acetyl-O-phosphotyrosyl-anchor3

The synthesis was carried out according to step 6b) of Example 3.

Characterization:

ESI-MS (calculated): (M+2H)²⁺ 730.0 (729.9)

### Example 6

### Combinatorial Synthesis on Anchor1

As an example of a combinatorial synthesis on anchor1, 9 LACs were prepared by combining of three amino acids (Ser, Lys, Leu) with three amines (3-amino-2,2-dimethyl-1-propanol=amine 1; (1S,2S)-2-benzyloxycyclohexylamine=amine2; (S)-3-methyl-2-butylamine=amine3), as shown in FIG. 4.

1) The amino acids a) Fmoc-L-Ser(tBu)-OH, b) Fmoc-L-Leu-OH and c) Fmoc-L-Lys(Boc)-OH were coupled to 90 mg resin each according to the standard cycle and the Fmoc group was each removed.

2) The coupling of the bromoacetic acid was carried out by incubating the three resins with 41.4 mg (0.3 mmol) bromoacetic acid and 45 μl (0.3 mmol) DIC each in 1 ml DMF for 30 min. Then the resin was washed five times with DMF and three times with DMSO.

3) The three resins were separated into three equal portions and the resulting nine resin portions were incubated as shown in FIG. 4 with 300 μl 2 M solutions of the three amines a) 3-amino-2,2-dimethyl-1-propanol=amine1; b) (1S,2S)-2-benzyloxycyclohexylamine=amine2 and c) (S)-3-methyl-2-butylamine=amine3 in DMSO for 2 hrs. Then the resins were washed five times each with DMSO and three times with DMF. The cleavage of the LAC was carried out according to the general protocol as described in step 6a of Example 3.

Characterization: ESI-MS (calculated)
Amine1-acetyl-Ser-anchor1: (M+H)⁺ 1195.1 (1194.8); (M+2H)²⁺ 598.6 (598.4)
Amine2-acetyl-Ser-anchor1: (M+H)⁺ 1296.9 (1296.9); (M+2H)²⁺ 649.7 (649.4)
Amine3-acetyl-Ser-anchor1: (M+H)⁺ 1179.1 (1178.8); (M+2H)²⁺ 590.6 (590.4)
Amine1-acetyl-Leu-anchor1: (M+H)⁺ 1221.2 (1220.9); (M+2H)²⁺ 611.7 (611.4)
Amine2-acetyl-Leu-anchor1: (M+H)⁺ 1322.8 (1322.9); (M+2H)²⁺ 662.7 (662.5)
Amine3-acetyl-Leu-anchor1: (M+H)⁺ 1205.2 (1204.9); (M+2H)²⁺ 603.6 (603.4)
Amine1-acetyl-Lys-anchor1: (M+H)⁺ 1236.2 (1235.9); (M+2H)²⁺ 619.2 (618.9)
Amine2-acetyl-Lys-anchor1: (M+H)⁺ 1337.9 (1337.9); (M+2H)²⁺ 670.3 (670.0)
Amine3-acetyl-Lys-anchor1: (M+H)⁺ 1220.3 (1219.9); (M+2H)²⁺ 611.2 (610.9)

### Example 7

### Synthesis of Ligand-Anchor Conjugates Based on Anchor6

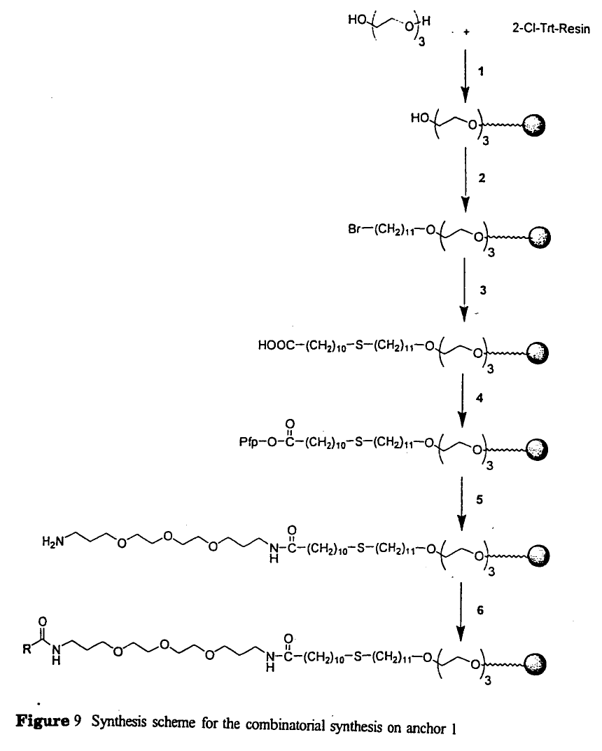

The synthesis of the LACs based on anchor6 was carried out on 2-chlorotrityl-chloride resin (FIG. 9).

1) 500 mg (1.35 mmol/g) of the 2-chlorotrityl-chloride resin were suspended in a round-bottomed flask fitted with a reflux cooler in 500 mg (3.33 mmol) triethylene glycol and 540 μl (6.67 mmol) pyridine in 5 ml tetrahydrofuran and stirred for 6 hrs at 60° C. Then the resin was transferred to a frit and washed five times with tetrahydrofuran.

2) 318 μl (1.35 mmol) 1,1-dibromoundecane were dissolved in 3 ml tetrahydrofuran and 142 mg (0.54 mmol) 18-crown-6 and 30 mg (0.54 mmol) KOH were added. 200 mg resin were added and the suspension was stirred for 16 hrs at room temperature. Then the resin was washed five times with tetrahydrofuran, five times with water, three times with DMF and three times with tetrahydrofuran.

3) 235 mg (1.08 mmol) 11-mercaptoundecanoic acid were dissolved in 4 ml tetrahydrofuran and 78 mg (3.24 mmol) LiOH, 137.4 mg (3.24 mmol) LiCl and 170 mg (1.02 mmol) KI were added. After addition of the resin the suspension was stirred under reflux for 16 hrs. Then the resin was washed five times with tetrahydrofuran, five times with water, three times with DMF, three times with dichloromethane, two times with hexane and dried in vacuo.

4) The activation of the carboxylic acid with simultaneous formation of the pentafluorophenyl ester was carried out according to step 4) of Example 3.

5) The coupling of 1,13-diamine-4,7,10-trioxatridecane was carried out according to step 5) of Example 3.

6) The acetylation was carried out according to step 6a) of Example 3.

General protocol for removing the LAC from the 2-chlorotrityl resin:

The resin was washed five times with dichloromethane and then incubated with 1 ml/(100 mg resin) 46/46/4/4 (v/v/v/v) trifluoroacetic acid/dichloromethane/water/triethylsilane for 20 min. The solution was removed from the resin and the resin was washed two times with the eluent. The pooled solutions were concentrated and the residue was purified by HPLC.

Characterization:

ESI-MS (calculated): (M+H)⁺ 765.8 (765.6)

### Example 8

### Synthesis of Ligand-Anchor Conjugates Based on Anchor7

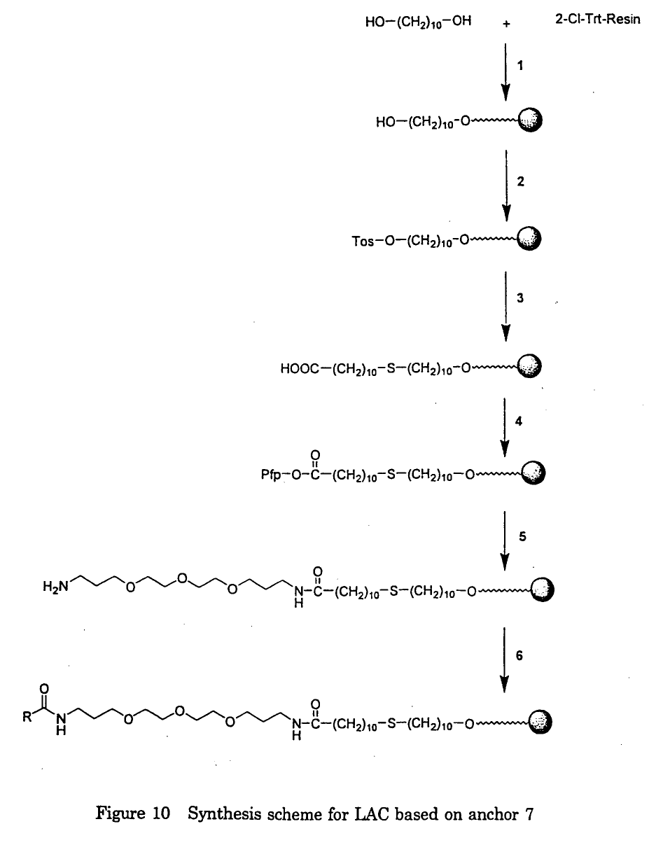

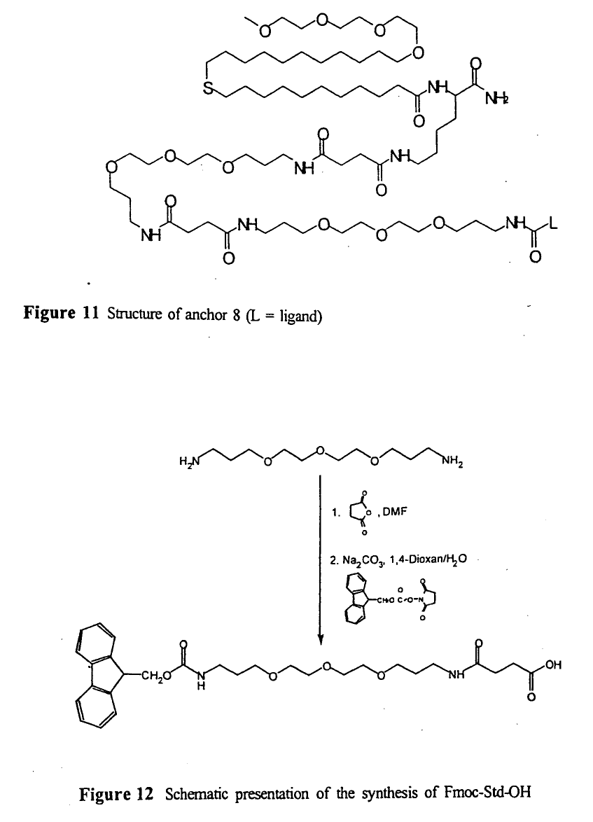

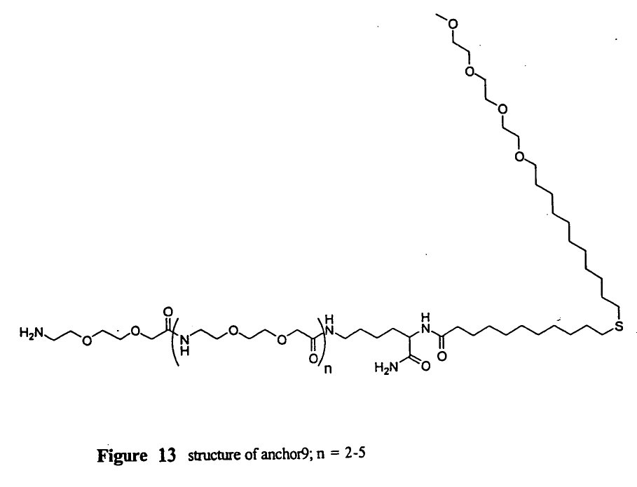

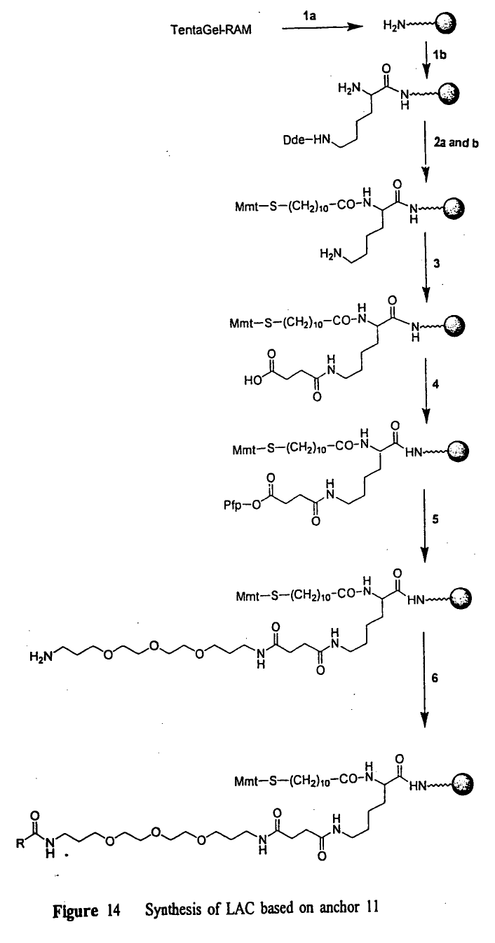

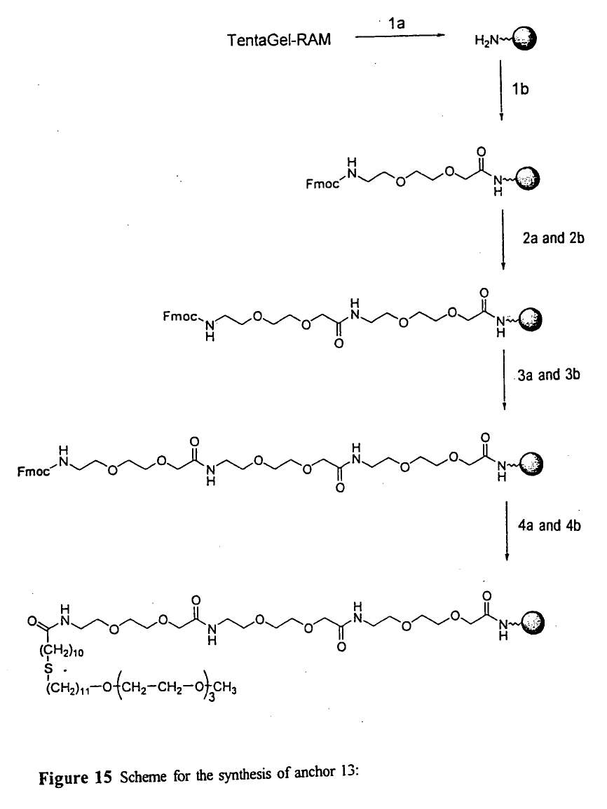

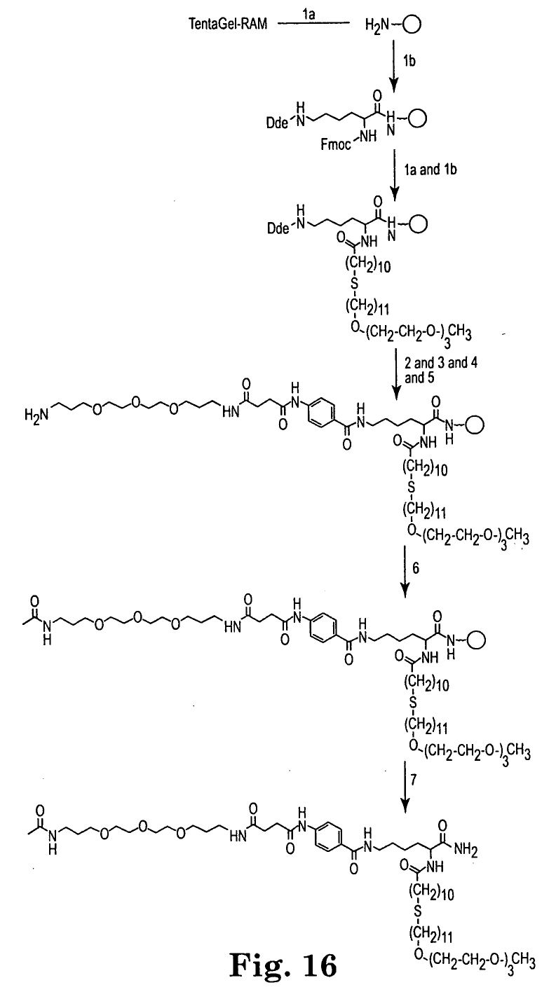

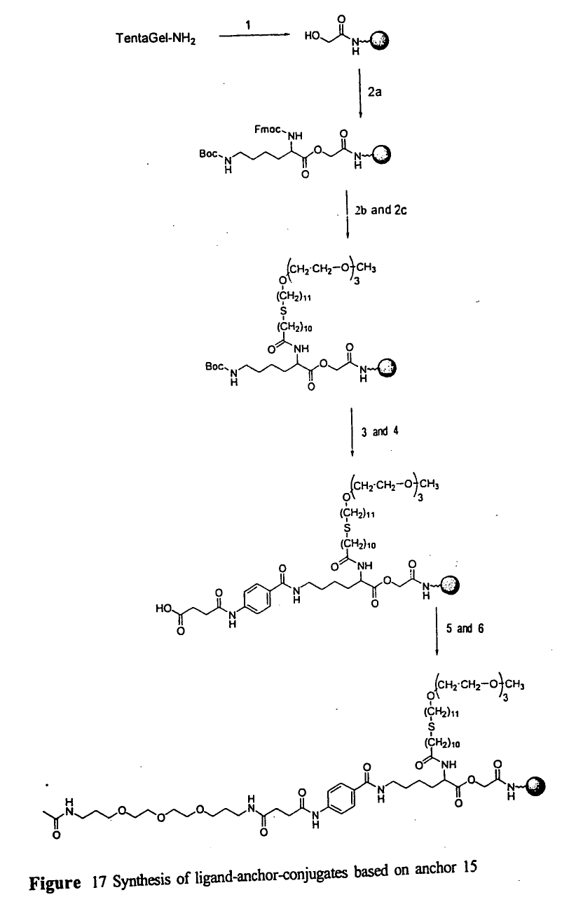

The synthesis scheme for LACs based on anchor7 is shown in FIG. 10.

1a) The 2-chlorotrityl-chloride resin was loaded with 1,10-decane diol according to step 1) of Example 7.

2) The free hydroxyl group was tosylated by incubating 500 mg resin with 515 mg (2.7 mmol) p-toluene sulfonylchloride and 425 μl (5.4 mmol) pyridine in 4 ml DCM. Then the resin was washed five times with dichloromethane and three times with tetrahydrofuran.

The following steps were carried out according to steps 3) to 6) of Example 7.

Characterization:

ESI-MS (calculated): (M+H)⁺ 619.8 (619.5)

### Example 9

### Synthesis of Ligand-Anchor Conjugates Based on Anchor8

The synthesis of the LACs based on anchor8 was carried out on TentaGel-RAM resin® in a polypropylene syringe with a polypropylene frit.

Standard cycle for the coupling of N-Fmoc-protected amino acids and of 24, 27, 30, 33-tetraoxa-12-thia-tetratriacontanoic acid of Example 1.

For cleavage of the Fmoc group 50 mg TentaGel-RAM® (0.24 mmol/g) resin was shaken for 20 min with 0.5 ml 20% piperidine in DMF. Then the resin was washed five times with DMF.

5 equivalents of the Fmoc amino acid (0.06 mmol) and 5 equivalents (9 mg, 0.06 mmol) 1-hydroxy-1H-benzotriazole (HOBt) were dissolved in 0.15 ml DMF and 5 equivalents (11 μl, 0.07 mmol) N,N′-diisopropylcarbodiimide (DIC) were added. This solution was added to the resin and the suspension was shaken for 60 min. Then the resin was washed five times with DMF.

Synthesis of Anchor8 (According to the Synthesis of Anchor1):

Coupling of Fmoc-Lys(Dde)-OH was carried out according to the standard cycle.

After Fmoc deprotection, 24,27,30,33-tetraoxa-12-thia-tetratriacontanoic acid was coupled according to the standard cycle with a coupling time of 6 hrs.

For cleavage of the Dde protecting group the resin was incubated four times for 3 min each with 2.5% hydrazine in DMF. Then the resin was washed five times with DMF.

After Dde cleavage N-fluorenylmethoxycarbonyl-N′succinyl-4,7,10-trioxatridecane-1,13-diamine (Fmoc-Std-OH), which was prepared in solution using succinic anhydride, 4,7,10-trioxa-1,13-diaminetridecane and 9-fluorenylmethyloxycarbonyl-N-hydroxysuccinimide (see Fig. below), were coupled according to the standard cycle for Fmoc amino acids (2 hrs). After Fmoc cleavage this coupling step was repeated.

Coupling of the Ligands

### a) Acetyl-anchor8

The amino group of anchor8 was acetylated by incubating 50 mg resin with a solution of 50 μl acetic anhydride and 25 μl pyridine in 300 μl DMF. Then the LAC was removed from the resin.

Characterization:

ESI-MS (calculated): (M+2H)²⁺ 655.0 (655.6)

### b) Acetyl-O-phospho-tyrosyl-anchor8

Fmoc-Tyr(PO₃H₂)—OH was coupled according to the general protocol by adding 2 equivalents of ethyl-diisopropylamine. After cleavage of the Fmoc protecting group, the free amino group was acetylated according to a). The cleavage of the LAC was carried out according to the general protocol.

Characterization:

ESI-MS (calculated): (M+2H)²⁺ 776.9 (776.5)

### c) Acetyl-Gly-Arg-Gly-Asp-Ser-anchor8

The coupling of the L amino acids was carried out according to the standard cycle using the following amino acid derivatives: Fmoc-Gly-OH, Fmoc-Ser(tBu)-OH, Fmoc-Asp(OtBu)-OH and Fmoc-Arg(Pbf)-OH. The acetylation was carried out according to a). The cleavage of the LAC was carried out according to the general protocol.

Characterization:

ESI-MS (calculated): (M+2H)²⁺ 892.1 (892.1)

### Example 10

### Synthesis of Anchor9-Based Ligand-Anchor Conjugates

Synthesis of Anchor9-1 (n=2) to 9-4 (n=5):

The synthesis of anchor9-1 to 9-4 was carried out as described for anchor8, the only difference being that instead of the two successive Fmoc-Std-OH couplings (n+1) successive couplings of Fmoc-8-amino-3,6-dioxa caprylic acid (Neosystem, Strasbourg) were carried out.

4) Coupling of the Ligands

### a) Acetyl-anchor9-1

The amino group of the anchor9-1 was acetylated by incubating 50 mg resin with a solution of 50 μl acetic anhydride and 25 μl pyridine in 300 μl DMF. Then the LAC was removed from the resin according to the general protocol.

Characterization:

ESI-MS (calculated): (M+2H)²⁺ 570.9 (570.4)

### b) Acetyl-O-phospho-tyrosyl-anchor9-1

Fmoc-Tyr(PO₃H₂)—OH was coupled according to the general protocol using 2 equivalents ethyl-diisopropylamine. After cleavage of the Fmoc protection group the free amino group was acetylated according to a). The cleavage of the LAC was carried out according to the general protocol.

Characterization:

ESI-MS (calculated): (M+2H)²⁺ 692.2 (691.9)

### c) Acetyl-Gly-Arg-Gly-Asp-Ser-anchor9-1

The coupling of the L amino acids was carried out according to the standard cycle using the following amino acid derivatives: Fmoc-Gly-OH, Fmoc-Ser(tBu)-OH, Fmoc-Asp(OtBu)-OH and Fmoc-Arg(Pbf)-OH. The acetylation was carried out according to a). The cleavage of the LAC was carried out according to the general protocol.

Characterization:

ESI-MS (calculated): (M+2H)²⁺ 807.1 (806.5)

### d) Acetyl-anchor9-2

The acetylation was carried out according to a). The cleavage from the resin was carried out according to the general protocol.

Characterization:

ESI-MS (calculated): (M+2H)²⁺ 643.9 (642.9)

### e) Acetyl-anchor9-3

The acetylation was carried out according to a). The cleavage from the resin was carried out according to the general protocol.

Characterization:

ESI-MS (calculated): (M+2H)²⁺ 715.6 (715.5)

### f) Acetyl-anchor9-4

The acetylation was carried out according to a). The cleavage from the resin was carried out according to the general protocol.

Characterization:

ESI-MS (calculated): (M+2H)²⁺ 789.5 (788.0)

### Example 11

### Synthesis of (Thiol Anchor) Ligand-Anchor Conjugates Based on Anchor 11

The synthesis of the LACs based on anchor 11 was carried out until step 1b and from step 2b onwards in the FIG. 14 below according to the synthesis steps 1a to 1b and 2b to 6b in Example 3.

2a) The coupling of S-Mmt-11-mercaptoundecanoic acid was carried out according to the standard cycle of Example 3.

11-Mercaptoundecanoic acid was protected with the Mmt protecting group according to M. Bodanszky, A. Bodanszky, The Practice of Peptide Synthesis, Springer Verlag, Berlin, 2^{nd} edition, 1994, page 68.

### 6a) Acetyl-anchor11

Characterization:

ESI-MS (calculated): (M+H)⁺ 690.7 (690.4).

### 6b) Acetyl-O-phospho-tyrosyl-anchor11

Characterization:

ESI-MS (calculated): (M+H)⁺ 933.8 (933.5).

### Example 12

### Synthesis of Ligand-Anchor Conjugates Based on Anchor12

Synthesis of Anchor12:

The synthesis of anchor12 was carried out as described for anchor10 up to the second coupling of Fmoc-8-amino-3,6-dioxa caprylic acid. After cleavage of the Fmoc protecting group 3,6,9-trioxaundecanediacid was coupled. This was carried out by incubating 100 mg resin with 30 mg 3,6,9-trioxaundecanediacid, 23 μl diisopropylcarbodiimide and 25 μl ethyl-diisopropylamine in 300 μl DMF for 90 min. Then the resin was washed five times with DMF.

### a) 2,4-Diamino-6-(hydroxymethyl)-pteridine—Anchor12

The free carboxylate group was converted to the Pfp ester according to Example 3, 4). The coupling of the ligand was carried out by shaking 50 mg resin with 12 mg 2,4-diamino-6-(hydroxymethyl)-pteridine, 15 μl N-methylimidazole in 250 μl DMF for 2 hrs at room temperature. The cleavage of the ligand-anchor conjugate was carried out according to the general protocol.

Characterization:

LC-MS (expected): [M+2]²⁺ 666.6 (666.4)

### Example 13

### Synthesis of Anchor 13 as Diluting Component

The synthesis of the LAC was carried out in a polypropylene syringe using a polypropylene frit on 50 mg (0.26 mmol/g) TentaGel-RAM® resin.

The cleavage of the Fmoc group and the coupling of Fmoc-8-amino-3,6-dioxa caprylic acid was carried out according to the standard cycle (see Example 3).

The coupling of 24,27,30,33-tetraoxa-12-thia-tetratriacontanoic acid was carried out according to the standard cycle with a coupling time of 90 minutes.

The cleavage of the anchor was carried out according to the general protocol for the cleavage of LAC (see Example 3).

Characterization:

ESI-MS (calculated): (M+H)⁺ 969.8 (968.6)

### Example 14

### Synthesis of Ligand-Anchor Conjugates Based on Anchor 14

The synthesis of the LAC was carried out in a polypropylene syringe with a polypropylene frit on 100 mg (0.26 mmol/g) TentaGel-RAM® resin.

1a) The cleavage of the Fmoc group was carried out according to the standard cycle (see Example 3).

1b) The coupling of Fmoc-Lys(Dde)-OH and of 24,27,30,33-tetraoxa-12-thia-tetratriacontanoic acid was likewise carried out according to the general protocol (see Example 3) with a coupling time of 90 min. For the coupling of Fmoc-Lys(Dde)-OH 5 equivalents 1-hydroxy-1H-benzotriazole were additionally added.

2) For the cleavage of the Dde protecting group the resin was incubated four times for 3 min each with 2% hydrazine in DMF. Then the resin was washed five times with DMF.

3) 6 equivalents of the 4-aminobenzoic acid (0.156 mmol) and 6 equivalents (24 mg, 0.156 mmol) 1-hydroxy-1H-benzotriazole (HOBt) were dissolved in 500 μl DMF and 6 equivalents (25 μl, 0.156 mmol) N,N′-diisopropylcarbodiimide (DIC) were added. This solution was added to the resin and the suspension was shaken for 90 min. Then the resin was washed twice with DMF and the coupling was once repeated. Then the resin was washed five times with DMF.

4) The coupling of the succinic acid was carried out by incubating the resin with 5 equivalents succinic anhydride and with 5 equivalents HOBt in 750 μl DMF overnight. Then the resin was washed five times with DMF.

5) For the coupling of 1,13-diamino-4,7,10-trioxatridecane first the pentafluorophenylester was prepared. This was carried out by incubating the resin with a solution of 200 μl (1.16 mmol) trifluoroacetic acid pentafluorophenylester and 100 μl (1.24 mmol) pyridine in 500 μl DMF for 2 hrs. Then the resin was washed five times with DMF.

The coupling of 1,13-diamino-4,7,10-trioxatridecane was carried out by incubating the resin with a solution of 500 μl 1,13-diamino-4,7,10-trioxatridecane and 50 mg HOBt in 500 μl DMF overnight. Then the resin was washed five times with DMF.

6) The amino group of 1,13-diamino-4,7,10-trioxatridecane was acetylated by incubating the resin with a solution of 50 μl acetic anhydride and 100 μl pyridine in 150 μl DMF.

7) The cleavage of the LAC was carried out according to the general protocol (see Example 3).

Characterization:

ESI-MS (calculated): (M+2H)²⁺ 564.2 (563.8)

### Example 15

### Synthesis of Ligand-Anchor Conjugates Based on Anchor 15

The synthesis of the LACs was carried out in a polypropylene syringe with a polypropylene frit on 200 mg (0.25 mmol/g) TentaGel-NH₂® resin.

1) The coupling of the glycolic acid was carried out by incubating the resin with a solution of 20 mg (0.25 mmol) glycolic acid, 39 mg (0.25 mmol) HOBt and 40 μl DIC in 7501 μl DMF for 2 hrs. Then the resin was washed five times with DMF.

2a) For the coupling of Fmoc-Lys(Boc)-OH 5 equivalents of Fmoc-Lys(Boc)-OH, 5 equivalents 1-hydroxy-1H-benzotriazole (HOBt), 5 equivalents N,N′-diisopropylcarbodiimide (DIC) and 5 equivalents N-methylimidazol (NMI) were dissolved in 750 μl DMF. This solution was added to the resin and the suspension was shaken for 90 min.

2b) The cleavage of the Fmoc group was carried out according to the standard cycle (see Example 3).

2c) The cleavage of the Fmoc group was carried out according to the standard cycle (see Example 3). The coupling of 24,27,30,33-tetraoxa-12-thia-tetratriacontanoic acid was carried out according to the general protocol (see Example 3) with a coupling time of 90 min.

2d) The resin was washed three times with dichloromethane (DCM). For the cleavage of the Boc protecting group the resin was incubated for 30 min with 750 μl 50% TFA in DCM. Then the resin was washed three times with DCM and five times with DMF.

3) 6 equivalents of the 4-aminobenzoic acid (0.156 mmol) and 6 equivalents (24 mg, 0.156 mmol) 1-hydroxy-1H-benzotriazole (HOBt) were dissolved in 750 μl DMF and 6 equivalents (25 μl, 0.156 mmol) N,N′-diisopropylcarbodiimide (DIC) were added. This solution was added to the resin and the suspension was shaken for 90 min. Then the resin was washed twice with DMF and the coupling was once repeated. Then the resin was washed five times with DMF.

4) The coupling of the succinic acid was carried out by incubating the resin with 5 equivalents succinic anhydride and with 5 equivalents HOBt in 750 μl DMF overnight. Then the resin was washed five times with DMF.

5) For the coupling of 1,13-diamino-4,7,10-trioxatridecane first the pentafluorophenylester was prepared. This was carried out by incubating the resin with a solution of 200 μl (1.16 mmol) trifluoroacetic acid pentafluorophenylester and 100 μl (1.24 mmol) pyridine in 500 μl DMF for 2 hrs. Then the resin was washed five times with DMF.

The coupling of 1,13-diamino-4,7,10-trioxatridecane was carried out by incubating the resin with a solution of 500 μl 1,13-diamino-4,7,10-trioxatridecane and 50 mg HOBt in 500 μl DMF overnight. Then the resin was washed five times with DMF.

6) The amino group of 1,13-diamino-4,7,10-trioxatridecane was acetylated by incubating the resin with a solution of 50 μl acetic anhydride and 100 μl pyridine in 150 μl DMF.

7) For cleavage of the LAC from the resin the resin was washed three times with ethanol and then incubated with 1 ml 0.085 M KOH in water for 1 h.

The solution was removed from the resin and the resin was washed with 850 μl 0.1 M HCl in water. The pooled solutions were lyophilized. The LAC was dissolved in 1/1 water/acetonitril and purified by HPLC.

Characterization:

ESI-MS (calculated): (M+2H)²⁺ 564.7 (564.3)

### Example 16

### Synthesis of Anchor-Ligand Conjugates Based on Anchors 17 and 18

1) Immobilisation of N-(N⁵-Fmoc-5-aminopentyl)-11-mercaptoundecane Amide on Chlorotrityl Resin

600 mg (1.15 mmol) of N-(N⁵-Fmoc-aminopentyl)-11-mercaptoundecane amide, obtainable from S-protected 11-mercaptoundecane amide and Fmoc-1,5-diaminopentane hydrochloride, were dissolved in 15 ml DMF and blended with 2 g methoxytritylchloride-resin (1,6 mmol) (Novabiochem). The suspension was carefully shaken for 1 h. Subsequently 500 μl pyridine were added and the suspension was shaken for a further 3 h. The resin was then washed five times with DMF, three times with DCM, two times with hexane and dried in vacuum. The loading of the The loading of the resin with N-(N⁵-Fmoc-5-aminopentyl)-11-mercaptoundecane amide was determined by means of Fmoc-analysis to be 0.35 mmol/g (yield 60% of the theoretical value).

2) General Protocol for the Coupling of Fmoc-8-amino-3,6-dioxa-octanoic Acid (Fmoc-Ado)

For the cleavage of the Fmoc-protecting group, 1 g of the loaded resin (0.35 mmol) was carefully stirred for 20 min in 15 ml ⅓ (v/v) piperidine/DMF and then six times washed with DMF. The coupling of Fmoc-8-amino-3,6-dioxaoctanoic acid was effected by incubating the resin for 4 h with a solution of 270 mg (0.70 mmol) Fmoc-8-amino-3,6-dioxa-octanoic acid, 270 mg (0.71 mmol) HATU and 250 μl (1.44 mmol) ethyldiisopropyl amine in 7 ml DMF. Subsequently the resin was washed five times with DMF, three times with dichloremethane and two times with hexane and dried. 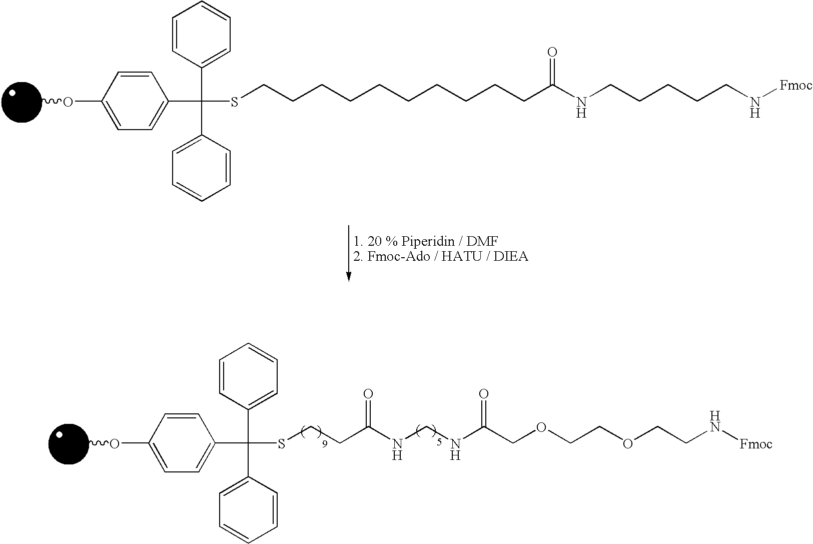
3) Synthesis of a Diluting Component

Fmoc-8-amino-3,6-dioxa-octanoic acid was coupled to 500 mg resin from 2) (0.175 mmol) as described in step 2) and then the Fmoc-protecting group was removed as described in step 2). The free amino groups were then acetylated by incubating the resin for 30 min with 10 ml 1/1/2 (v/v/v) acetic acid anhydride/pyridine/DMF. Then the resin was washed five times with DMF and three times with dichloromethane. The cleavage of the product from the resin was effected with 2/18/1 (v/v/v) trifluoro acetic acid/dichloromethane/triethylsilane. The product was purified by preparative RP-HPLC and analyzed by means of LC/MS.

LC-MS (calc.): [M+H]+635.5 (635.4), [M+Na]⁺ 657.5 (657.4) 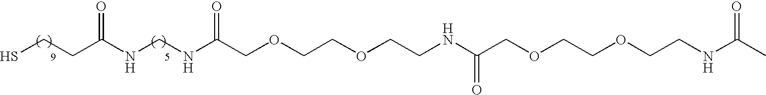
4) Synthesis of the Anchor 17

For the synthesis of the anchor Fmoc-8-amino-3,6-dioxa-octanoic acid was twice coupled to the resin obtained in 2) as described under 2) and then the Fmoc-protecting group was removed. The resin was then washed three times with dichloromethane and two times with hexane and dried in vacuum. 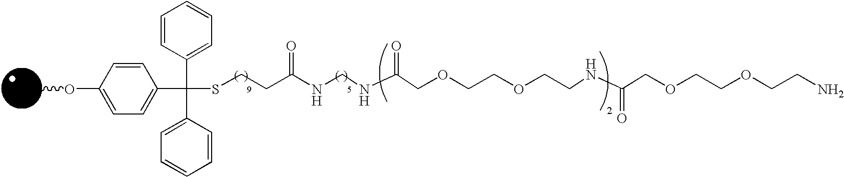
5) General Protocol for the Coupling of Carboxylic Acid Ligands to Anchor 17 Taking p-amino Benzoic Acid as an Example

For coupling a carboxylic acid to the anchor the resin obtained in 4) was incubated for 1 h with a solution of 4 eq. carboxylic acid, 4 eq. diisopropylcarbodiimide and 4 eq. 1-hydroxy-benzotriazole in DMF (c=0.15 M). The resin was then washed five times with DMF and three times with dichloromethane. The cleavage of the product from the resin was effected by incubating the resin for 1 h with 18/1//1 (v/v/v) trifluoro acetic acid/water/triethylsilane. The product was purified by preparative RP-HPLC and analyzed by means of LC/MS.

### Example: p-aminobenzoic Acid

LC-MS (calc.): [M+H]+872.3 (872.2) [(M+2H)/2]²⁺ 436.3 (436.6) 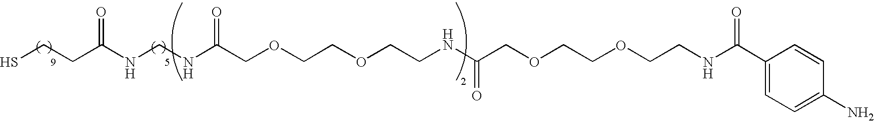
6) Coupling of amines to the anchor taking N 6-(6-aminohexyl)adenosine-2′,5′-diphosphate as an example

Anchor 18 was obtained by incubating the resin obtained in 4) with 1 ml (100 mg resin) 2/1/17 (w/v/v) succinic acid anhydride/pyridine/DMF and coupling for 60 min. The resin was then washed five times with DMF.

The pentafluorophenyl ester of the free carboxylic acid was then prepared by incubating 100 mg resind for 1 h with a solution of 100 μl (0.58 mmol) trifluoro acetic acid pentafluoro phenylester and 50 μl (0.62 mmol) pyridine in 500 μl DMF. The resin was then washed five times with DMF.

The coupling of the amine to the anchor was effected by incubating the resin with 500 μl/(100 mg resin) of a solution of 0.1 M amine, 0.1 M ethyl-diisopropyl amine and 0.1 M 1-hydroxy-benzotriazole in DMSO.

The cleavage of the product from the resin was effected by incubating the resin for 1 h with 18/1//1 (v/v/v) trifluoro acetic acid/water/triethylsilane. The product was purified by preparative RP-HPLC and analyzed by means of LC/MS.

### Example: N⁶-(6-aminohexyl)adenosine-2′,5′-diphosphate

LC-MS (calc.): [(M+2H)/2]²⁺ 674.0 (674.2)

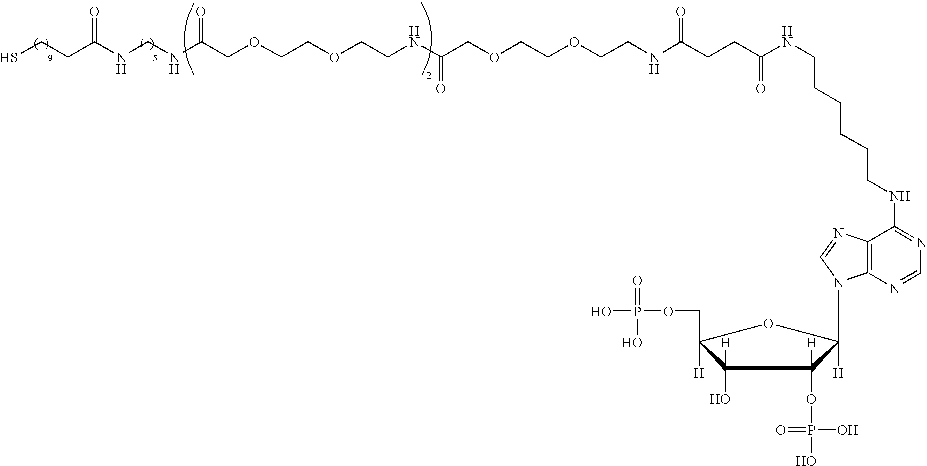

### Examples of Use

### Example 17

FIG. 24 illustrates the CCD picture of a section of four fields of a gold-coated carrier plate as a sensor surface as schematically shown in FIG. 21. The carrier plate altogether comprises 9216 fields. A chemical luminescence reaction is used to detect fields on which a specific ligand receptor binding has taken place. The overcoat layer (1) cannot be seen in this Fig.

Two fields 500×500 μm in size (field 1a and 2a in FIG. 24) of the carrier plate (5) are each covered with 0.1 μl of a solution of N-acetylphosphotyrosine which is covalently coupled to a sulfide anchor to form an amide binding (LAK 1, FIG. 18).

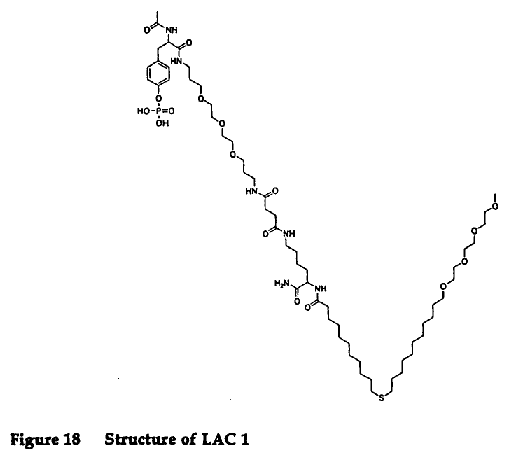

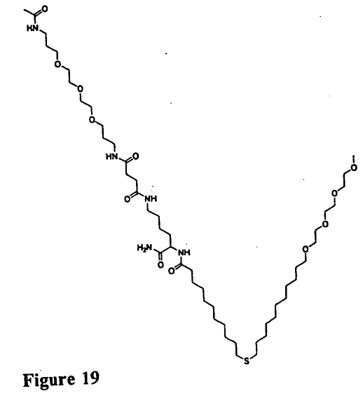

0.1 μl of a solution of an identical anchor molecule which has an acetyl group (non-ligand) at its amino N atom and, thus, should not bind to proteins are placed on two further fields (1b and 2b) (diluent 1, FIG. 19).

The concentration of the anchor-ligand conjugate or of the anchor molecule is 1 mM in 20% HBS, 30% ethylene glycol, 50% acetonitrile pH 7.2. The solutions dry up on the gold field. The plate is then immersed into a solution of 150 mM NaCl, 5 mg/ml BSA 0.5% (w/v) Tween-20 and 50 mM Tris/HCl pH 7.3 to saturate gold areas which might not have been covered yet and incubated for 10 h at 4° C.

The plate is then immersed into a solution of 8.6 nM anti-phosphotyrosine (Sigma) antibody in 0.5% (w/v) Tween-20 and 50 mM Tris/HCl pH 7.3 and incubated for 4 h at 4° C. The anti-phosphotyrosine antibody serves as the receptor in the sense of the invention. After a subsequent short washing step in 0.5% (w/v) Tween-20 and 50 mM Tris/HCl pH 7.3 the plate is placed into a solution of 0.04 U/ml anti-mouse-Fab-fragment-alkaline phosphatase conjugate (Boehringer Manheim) and incubated for another 4 h at 4° C. The plate is then washed in TBS and for the detection of the binding the plate is placed into the ELISA-substrate BM Chemiluminescence Elisa Substrate AP and the luminescence reaction which occurs on the individual sensing fields is observed by means of a Lumi-Imager (Boehringer Mannheim) based on CCD. FIG. 24 shows that the gold fields which are coated with an anchor bearing a ligand immobilise anti-phosphotyrosine antibodies. The specificity of the reaction is evident from the fact that fields which had only be coated with acetylated anchor molecules do not immobilise any antibodies.

### Example 18

Mixtures of phosphotyrosine anchor conjugate and acetylated anchor (cf. Example 17) are applied onto twelve fields of a carrier plate as used in Example 17 in varying ratios. The total concentration of molecules bearing anchors was kept at 1 mM. FIG. 25 depicts the CCD picture of these fields during the luminescence reaction. The ratio of anchor bearing ligands to acetylated anchor was varied from top to bottom. The substances were applied at mixing ratios of 1:0, 1:1, 1:10, 1:100, 1:1000 and 1:10000. Afterwards the carrier plate was treated according to the one in example 1. It can be seen that the signal intensity increases as the proportion of phosphotyrosine anchor conjugate increases. The high sensitivity is evident from the fact that even a ratio of phosphotyrosine anchor conjugate to acetylated anchor of 1:10000 will still give a signal which differs from that of a acetyl anchor surface. Resist 1 of the carrier plate has the advantageous property that under the test conditions neither anchor molecules will be present nor unspecific protein binding will occur and, thus, the detection fields are clearly separate from each other.

### Example 19

Under the conditions described above a carrier plate as used in Example 17 comprising 24×32=768 fields was coated with 487 different ligand-anchor conjugates.

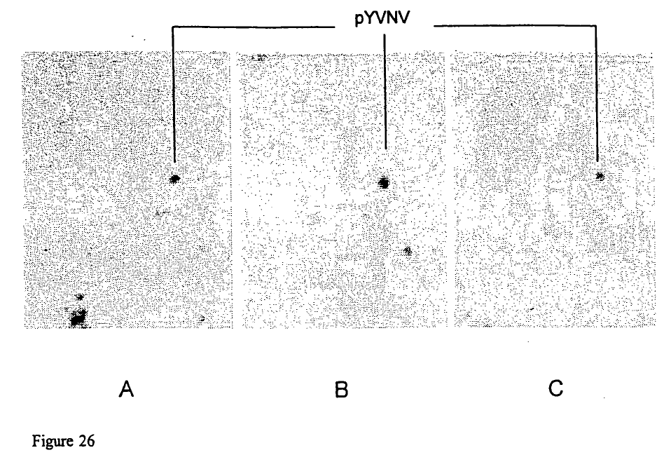

FIG. 26A-C illustrates the parallel luminescence measurement by means of a CCD camera at varying concentrations after the plate had been treated as follows: After saturating the carrier plate as described in Example 17 the carrier plate is incubated for 4 hours in a 10 nM solution of a Grb2-SH2-protein A fusion protein (Sigma) in 150 mM NaCl, 5 mg/ml BSA, 0.5% (w/v) Tween-20 and 50 mM Tris/HCl pH 7.3. After a short washing step in 150 mM NaCl, 5 mg/ml BSA, 0.5% (w/v) Tween-20 and 50 mM Tris/HCl pH 7.3 the carrier plate is incubated for 90 min in a 1:5000 diluted anti goat-AP conjugated antibody solution (Sigma) with 150 mM NaCl, 5 mg/ml BSA, 0.5% (w/v) Tween-20 and 50 mM Tris/HCl pH 7.3. After washing it twice in TBS binding reactions occurring on the carrier plate are detected by means of a chemical luminescence reaction in BM Chemiluminescence Elisa Substrate AP observed in the Lumi-Imager (Boehringer Mannheim). The concentration of the anchor molecules was kept at 1 mM and the ratio of anchor bearing ligands to acetylated anchor was 1:1 in FIG. 26A, 1:5 in FIG. 26B and 1:10 in FIG. 26C. The marked field in FIG. 26A-C illustrates the strong ligand receptor interaction between the ligand pYVNV and the enzyme. Moreover, some other ligands which are also specifically immobilized to the protein can be recognizedm the interaction of which, however, is by far less strong.

In accordance with Example 18 the intensity of the blackening is to be attributed to the concentration of ligands and receptors as well as to the strength of the ligand receptor interaction. The following ligands (amino carboxylic acids, carboxylic aicdsor amines) were presented on the fields of the carrier plate:
propargylamine, cyclopropylamine, propylamine, ethylenediamine, ethanolamine, imidazole, 3-aminopropionitrile, pyrrolidine, glyoxylic acid monohydrate, acetic hydrazide, 1-glycine, glycolic acid, pyridine, 1-methylimidazol, cyanoacetic acid, cyclopropanecarboxylic acid, (s)-(+)-3-methyl-2-butylamine, pyruvic acid, n,n-dimethylethylenediamine, n,n′-dimethylethylenediamine, 1-alanine, beta-1-alanine, d-alanine, beta-alanine, sarcosine, (r)-2-amino-1-butanol, 2-amino-1,3-propanediol, aniline, 3-aminopyridine, 4-pentynoic acid, 4-pentenoic acid, alpha-beta-dehyro-2-aminobutyric acid, aminocyclopropylcarboxylic acid, 3-amino-1-propanol vinyl ether, (r)-(−)-tetrahydrofurfurylamine, (s)-(+)-prolinol, (r)-3,3-dimethyl-2-butylamine, 1,5-diaminopentane, gamma-aminobutyric acid, 2-aminobutyric acid, 2-aminoisobutyric acid, 3-amino-2,2-dimethyl-1-propanol, thiomorpholine, 1-2,3-diaminopropionic acid, d-serine, 1-serine, 2-(2-aminoethoxy)ethanol, (methylthio)acetic acid, benzylamine, 3-chloropropionic acid, 4-aminophenol, histamine, quinuclidine, exo-2-aminonorbornane, cyclopentanecarboxylic acid, trans-1,4-diaminocyclohexane, 1-proline, d-proline, 1-allylglycine, 1-amino-1-cyclopentanemethanol, tetrahydro-2-furoic acid, 3,3-dimethylbutyric acid, succinamic acid, 1-valine, 1-leucinol, hydantoic acid, 1-threonine, d-threonine, (s)-(−)-alpha-methylbenzylamine, 2-(2-aminoethyl)pyridine, 5-amino-o-cresol, p-anisidine, pyrazinecarboxylic acid, 1-(3-aminopropyl)imidazole, tropane, cyclooctylamine, 1-alpha-aminocaprolactam, 5-oxo-1-proline, isonipecotic acid, 1-pipecolic acid, 1,4,7-triazacyclononane, octylamine, dibutylamine, 4-methyl-2-oxovaleric acid, 1-aspartic acid, 1-asparagine, 1-leucine, 6-aminohexanoic acid, 1-isoleucine, 1-alpha-t-butylglycine, d-leucine, z-beta-alanine, 1-asparagine, 1-ornithine, 5-aminoindole, 1-aspartic acid, d-aspartic acid, 1-thiazolidine-4-carboxylic acid, 4-aminobenzoic acid, 3-(2-furyl)acrylic acid, 3-thiopheneacetic acid, cycloheptanecarboxylic acid, 3,5-difluorobenzylamine, 1,4-dioxa-8-azaspiro[4,5]-decane, n-cyclohexylethanolamine, caprylic acid, 1-glutamine, d-glutamine, 1-lysine, d-glutamic acid, 1-glutamic acid, 4-cyanobenzoic acid, (s)-1,2,3,4-tetrahydro-1-naphthylamine, 2,2,3,3,3-pentafluoropropylamine, (1s,2r)-(−)-cis-1-amino-2-indanol, 1-methionine, d-methionine, 4-carboxybenzaldehyde, 3-phenylpropionic acid, 4′-aminoacetanilide, piperonylamine, 1-phenylglycine, d-phenylglycine, 4-(aminomethyl)benzoic acid, 1-adamantanamine, 4-(hydroxymethyl)benzoic acid, (−)-cis-myrtanylamine, (1r,2r,3r,5s)-(−)-isopinocampheylamine, (r)-(+)-bomylamine, 1,3,3-trimethyl-6-azabicyclo[3,2,1]octane, 3,5-dihydroxybenzoic acid, 2-norbornaneacetic acid, 1-2-furylalanine, 1-histidine, d-histidine, 1-cyclohexylglycine, ethyl pipecolinate, 5-amino-1-naphthol, tryptamine, 4-aminobutyraldehyde diethyl acetal, 2-benzofurancarboxylic acid, 1-indoline-2-carboxylic acid, d-phenylalanine, 1-phenylalanine, 4-dimethylaminobenzoic acid, 1-methionine-sulfoxide, 3-(4-hydroxyphenyl)-propionic acid, dl-atrolactic acid hemihydrate, 4-sulfamoylbutyric acid, vanillic acid, 4-aminobiphenyl, (r)-(+)-citronellic acid, 4-chlorophenylacetic acid, 1-3-thienylalanine, 1-cyclohexylalanine, d-cyclohexylalanine, (s)-(−)-1-(1-naphthyl)-ethylamine, 2-chloro-6-methylnicotinic acid, 1-arginine, d-arginine, 1-4-thiazolylalanine, 3-pyridylacetic acid hydrochloride, 3-indolylacetic acid, 7-amino-4-methylcoumarin, 1-citrulline, 4-benzylpiperidine, 2,4-dichlorobenzylamine, 4-amino-n-methylphthalimide, (−)-cotinine, 1-tetrahydroisoquinolinecarboxylic acid, 4-acetamidobenzoic acid, (r)-(−)-2-benzylamino-1-butanol, 4-pentyloxyaniline, o-acetylsalicylic acid, 4-nitrophenylacetic acid, 2-nitrophenylacetic acid, 2-methyl-6-nitrobenzoic acid, 1-tyrosine, d-tyrosine, 1-methionine(o2), 3-(diethylamino)propionic acid hydrochloride, 4-nitroanthranilic acid, 2,6-dimethoxybenzoic acid, 3,5-dimethoxybenzoic acid, 3,4-dihydroxyhydrocinnamic acid, 2-(4-hydroxyphenoxy)propionic acid, 2-methoxyphenoxyacetic acid, 4-hydroxy-3-methoxyphenylacetic acid, 4-(ethylthio)benzoic acid, s-benzylthioglycolic acid, 4-(methylthio)phenylacetic acid, 2-chlorocinnamic acid, 3-chlorocinnamic acid, gamma-maleimidobutyric acid, 2,6-dimethoxynicotinic acid, 1-4-fluorophenylalanine, 1-2-fluorophenylalanine, (r)-(−)-epinephrine, cyclododecylamine, trans-2,5-difluorocinnamic acid, dl-3,4-dihydroxymandelic acid, thymine-1-acetic acid, cis-pinonic acid, 1,2-bis(4-pyridyl)ethane, 4-tert-butylcyclohexanecarboxylic acid, n,n-diethylnipecotamide, 3,4-difluorohydrocinnamic acid, 2-naphthylacetic acid, 3-carboxy-proxyl, 4-chloro-o-anisic acid, 4-chlorophenoxyacetic acid, 3-chloro-4-hydroxyphenylacetic acid, 5-chloro-2-methoxybenzoic acid, 4-chloro-dl-mandelic acid, 4-(pyrrol-1-yl)benzoic acid, 4-(difluoromethoxy)benzoic acid, gallic acid monohydrate, 2,4,6-trihydroxybenzoic acid monohydrate, 6-hydroxy-2-naphthoic acid, suberic acid monomethyl ester, 2-hydroxydecanoic acid, 2-chloro-6-fluorophenylacetic acid, alpha-cyano-3-hydroxycinnamic acid, indole-3-glyoxylic acid, 8-hydroxyquinoline-2-carboxylic acid, 2-methyl-3-indoleacetic acid, 4-(trifluoromethyl)benzoic acid, coumarin-3-carboxylic acid, 3-hydroxy-2-quinoxalinecarboxylic acid, 4-fluoro-1-naphthoic acid, 1-phenyl-1-cyclopentanecarboxylic acid, p-toluenesulonyl chloride, 5-bromo-2-furoic acid, 2,5-dichlorobenzoic acid, 3,4-dichlorobenzoic acid, 5-methoxyindole-2-carboxylic acid, isoquinoline-3-carboxylic acid hydrate, 1-styrylalanine, 4-(dimethylamino)cinnamic acid, 4-oxo-2-thioxo-3-thiazolidinylacetic acid, 1-ethyl-3-(3-dimethylaminopropyl)carbodiimide hydrochloride, 5,6-dichloronicotinic acid, 2,6-dichloronicotinic acid, 2,6-dichloropyridine-4-carboxylic acid, trimellitic anhydride, d-(−)-quinic acid, trans-3,4-methylenedioxycinnamic acid, 7-methoxybenzofuran-2-carboxylic acid, trans-5-acetoxy-1,3-oxathiolane-2-carboxylic acid, 4-benzoylbutyric acid, 4-pentylbenzoic acid, 6-phenylhexanoic acid, 2-chloro-4,5-difluorobenzoic acid, 4-chloro-2,5-difluorobenzoic acid, 5-fluoroindole-3-acetic acid, n-formyl-dl-phenylalanine, 4-diethylaminobenzoic acid, 2-aminoanthracene, d-glucuronic acid, trans-ferulic acid, (s)-(+)-o-acetylmandelic acid, 4-aminohippuric acid, 1-adamantaneacetic acid, 6-bromohexanoic acid, alpha-hydroxyhippuric acid, n-[3-(2-furylacryloyl)]-glycine, 1-methyl 2-aminoterephthalate, 1-serine(bzl), 3,3,3-trifluoro-2-(trifluoromethyl)propionic acid, diethylphosphonoacetic acid, d-gluconic acid, 3-(4-fluorobenzoyl)propionic acid, 2,5-dimethoxyphenylacetic acid, mono-methyl cis-5-norbornene-endo-2,3-dicarboxylate, 4-hydroxy-3-nitrophenylacetic acid, 3-methoxy-4-nitrobenzoic acid, 5-methoxy-2-nitrobenzoic acid, 3,4,5-trimethoxybenzylamine, dl-4-hydroxy-3-methoxymandelic acid, (−)-camphanic acid, (1r)-(+)-camphanic acid, 2-methoxy-4-(methylthio)benzoic acid, cis-5-dodecenoic acid, 4-amino-5-carboxy-2-ethyl-mercaptopyrimidine, 4-aminocinnamic acid hydrochloride, dl-3-(4-hydroxyphenyl)lactic acid hydrate, 4-(methylsulfonyl)benzoic acid, 4-carboxy-2,2,6,6-tetramethylpiperidine 1-oxyl, 2-butyloctanoic acid, trans-2-chloro-6-fluorocinnamic acid, 4-chloro-o-tolyloxyacetic acid, 2-bromobenzoic acid, 4-carboxybenzenesulfonamide, 2-(2-aminothiazole-4-yl)-2-methoxyiminoacetic acid, I-(n-t-amino)-cyclopropanecarboxylic acid, 2-chloro-3-nitrobenzoic acid, 4-chloro-3-nitrobenzoic acid, 2-chloro-4-nitrobenzoic acid, 4-chloro-2-nitrobenzoic acid, 4-amino-5-chloro-2-methoxybenzoic acid, 5-bromonicotinic acid, 6-bromopicolinic acid, 2-methyl-5-phenylfuran-3-carboxylic acid, tributyl phosphine, 2-chloro-5-(methylthio)benzoic acid, 4,5-difluoro-2-nitrobenzoic acid, 2-hydroxy-5-(pyrrol-1-yl)benzoic acid, indole-3-butyric acid, 2-(trifluoromethyl)phenylacetic acid, 3-(trifluoromethyl)phenylacetic acid, 4-(trifluoromethyl)phenylacetic acid, 3,7-dihydroxy-2-naphthoic acid, 6-methylchromone-2-carboxylic acid, 1-tryptophan, d-tryptophan, 2,6-dichlorophenylacetic acid, 3,4-dichlorophenylacetic acid, 3-(trifluoromethyl)anthranilic acid, alpha-acetamidocinnamic acid, 5-methoxyindole-3-acetic acid, dl-indole-3-lactic acid, (1s,2s)-(−)-2-benzyloxycyclohexylamine, 3,5-dichloroanthranilic acid, chloramben, s-(+)-ibuprofen, dl-thioctic acid, 3,5-dichloro 4-hydroxybenzoic acid, 5-bromothiophene-2-carboxylic acid, 2,3,5,6-tetrafluoro-p-toluic acid, 2-fluoro-3-(trifluoromethyl)benzoic acid, 3-fluoro-4-(trifluoromethyl)benzoic acid, 5-azido-2-nitrobenzoic acid, trans-2,3-dimethoxycinnamic acid, n-(4-aminobenzoyl)-beta-alanine, 4-butoxyphenylacetic acid, 2-(2-aminophenyl)indole, 2-amino-3,4,5,6-tetrafluorobenzoic acid, 2-nitrophenylpyruvic acid, z-glycine, 4-(4-nitrophenyl)butyric acid, s-(−)-2-[(phenylamino)carbonyloxy]propionic acid, 1-threonine(bzl), 2,6-dichloro-5-fluoro-3-pyridinecarboxylic acid, trimesic acid, (4-formyl-3-methoxy-phenoxy)acetic acid, (e)-5-(2-carboxyvinyl)-2,4-dimethoxypyrimidine, 1-phenylalanine(4-no2), 2-oxo-6-pentyl-2h-pyran-3-carboxylic acid, n,n-bis(2-hydroxyethyl)-isonicotinamide, (+/−)-jasmonic acid, epsilon-maleimidocaproic acid, (s)-(−)-n-benzyl-1-phenylethylamine, 2,4-dinitrobenzoic acid, 2,4,5-trimethoxybenzoic acid, 3,4,5-trimethoxybenzoic acid, s-(thiobenzoyl)thioglycolic acid, 4-iodobutyric acid, 3-phenoxybenzoic acid, 4-(4-hydroxyphenyl)benzoic acid, d-desthiobiotin, (−)-menthoxyacetic acid, 2-(o-chlorophenoxy)-2-methyl-propionic acid, 4-bromophenylacetic acid, 3-bromo-4-methylbenzoic acid, 3-bromophenylacetic acid, [1 r-(1alpha,2beta,3alpha)]-(+)-3-methyl-2-(nitromethyl)-5-oxocyclopentaneacetic acid, 1-aspartic acid(ochx), 1-1-naphthylalanine, 2-(trifluoromethyl)cinnamic acid, monomethyl sebacate, 5-aminovaleric acid, o-carboxyphenyl phosphate, 4-(trifluoromethyl)hydrocinnamic acid, mono-ethyl(r)-3-acetoxyglutarate, beta-(naphthylmercapto)acetic acid, 3-bromo-4-fluorobenzoic acid, 3-phthalimido-propionic acid, 1-arginine(no2), cis-(1s,2r)-(−)-2-benzylaminocyclohexanemethanol, 7-hydroxycoumarin-4-acetic acid, 2-sulfobenzoic acid hydrate, 5-methoxy-1-indanone-3-acetic acid, 4,7,10-trioxa-1,13-tridecanediamine, 2,4-dichlorophenoxyacetic acid, (s)-(+)-2-oxo-4-phenyl-3-oxazolidineacetic acid, (s)-(−)-n-(1-phenylethyl)succinamic acid, 3-(trifluoromethylthio)benzoic acid, 5-(4-chlorophenyl)-2-furoic acid, 8-bromooctanoic acid, 1-aspartic acid(obzl), n-acetyl-1-tyrosine, 2-nitro-5-thiocyanatobenzoic acid, 9-fluorenone-4-carboxylic acid, fluorene-9-acetic acid, 2-chloro-5-(trifluoromethyl)benzoic acid, 1-(4-chlorophenyl)-1-cyclopentanecarboxylic acid, 3,5-diaminobenzoic acid dihydrochloride, n-acetyl-4-fluoro-dl-phenylalanine, 2,4,6-trichlorobenzoic acid, 2,3,4,5,6-pentafluorophenylacetic acid, 2,4-dinitrophenylacetic acid, 3,4,5-trimethoxyphenylacetic acid, xanthene-9-carboxylic acid, (r)-(+)-3-hydroxy-5-oxo-1-cyclopentene-1-heptanoic acid, 2-bibenzylcarboxylic acid, 2,2-diphenylpropionic acid, 4-bromocinnamic acid, 4-carboxybenzenesulfonazide, 3-benzoyl-2-pyridinecarboxylic acid, trans-4-chloro-3-nitrocinnamic acid, 2,3,5,6-tetrafluoro-4-hydroxybenzoic acid hydrate, 3,5-dinitrosalicylic acid, (z)-(2-(formamido)thiazol-4-yl)(methoxyimino)acetic acid, 1-glutamic acid gamma-cyclohexyl ester, mono-2-(methacryloyloxy)ethyl succinate, naproxen, 1-lysine(alloc)-oh, 4-bromomandelic acid, 2-bromo-5-methoxybenzoic acid, 1-hydroxyproline, 6-(amino)-hexanoic acid, n-tert-butoxycarbonyl-1-leucine, 4-bromo-3,5-dihydroxybenzoic acid, n-(4-carboxy-3-hydroxyphenyl)maleimide, 5-(2-nitrophenyl)-2-furoic acid, 5-(3-nitrophenyl)-2-furoic acid, n-phthaloyl-dl-alpha-aminobutyric acid, 1-thiazolidine-4-carboxylic acid, (s)-(−)-alpha-methoxy-alpha-(trifluoromethyl)phenylacetic acid, 7-carboxymethoxy-4-methylcoumarin, 3,5-di-tert-butylbenzoic acid, 2-(2-chloroacetamido)4-thiazoleacetic acid, 5-bromoorotic acid, 2-nitro-alpha,alpha,alpha-trifluoro-p-toluic acid, benzoyl-dl-leucine, 1-glutamic acid(obzl), n,n′-dibenzylethylenediamine, 1-biphenylalanine, diphenic acid, 1-4-bromophenylalanine, pindolol, 1-leucine-4-nitroanilide, alpha, alpha-diphenyl-1-prolinol, 1-pentafluorophenylalanine, 1-phosphotyrosine, 4-iodophenylacetic acid, 1-benzoylphenylalanine, methyl red, 1-tyrosine(bzl), pentafluorophenyl trifluoroacetate, 1-lysine(z), r-(+)-1,1′-binaphtyl-2,2′-diamine, (+)-dehydroabietylamine, n-(4-amino-2-methylphenyl)-4-chlorophthalimide, 1-pyrenebutyric acid, atropin, 1-phenylalanine(4-i), 4-(2,4-di-tert-amylphenoxy)butylamine, 1-diaminopropionic acid(ivdde), 1-lysine(dde), 1-lysine(2-cl-z)-oh, 1-tyrosine(2,6-cl2-bzl), 4,4′-(9-fluorenylidene)-dianiline, 1-hydroxyproline, 4′-carboxy-benzo-18-crown-6, cholic acid as well as compounds having the following structure:

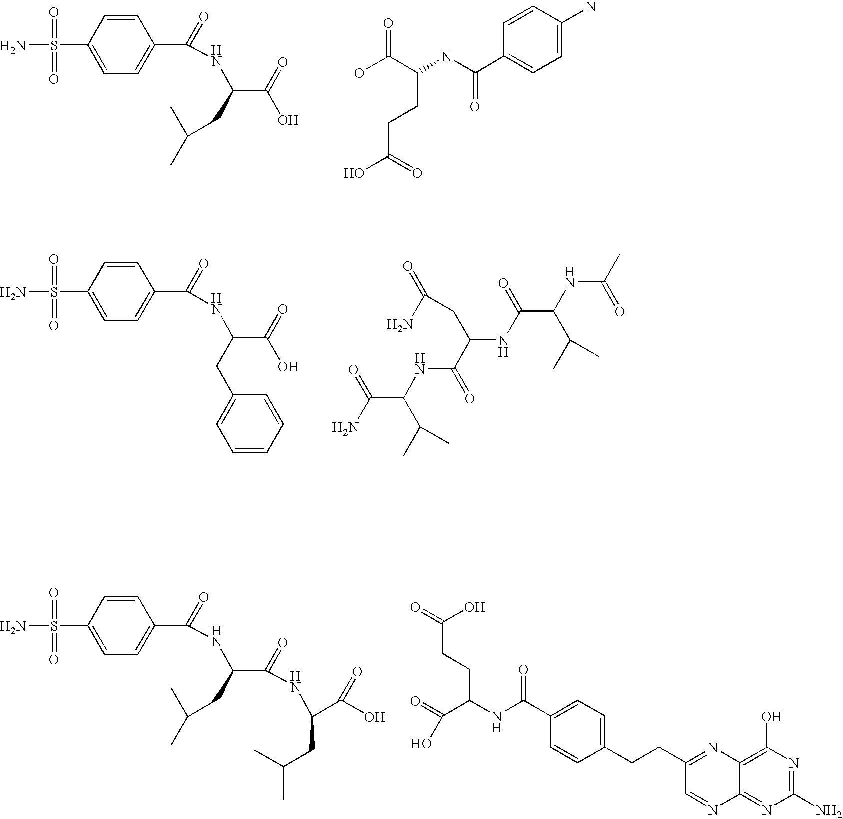

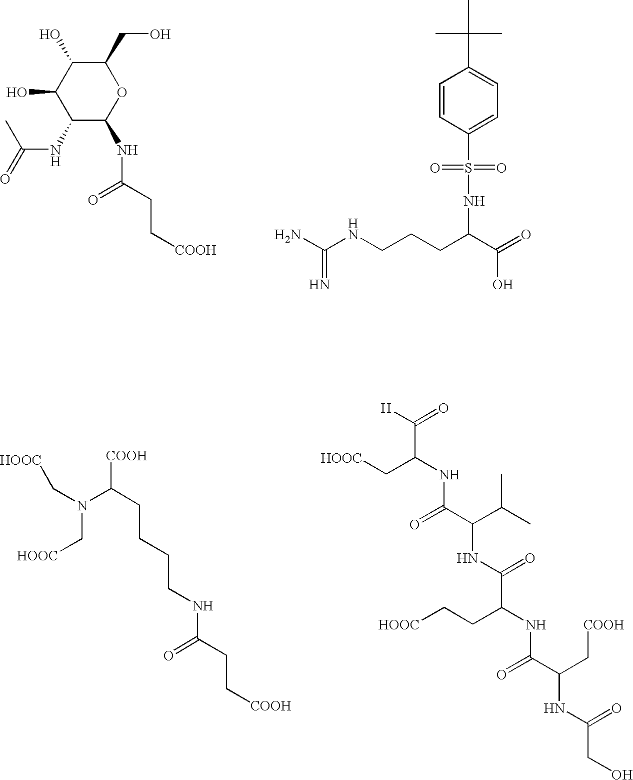

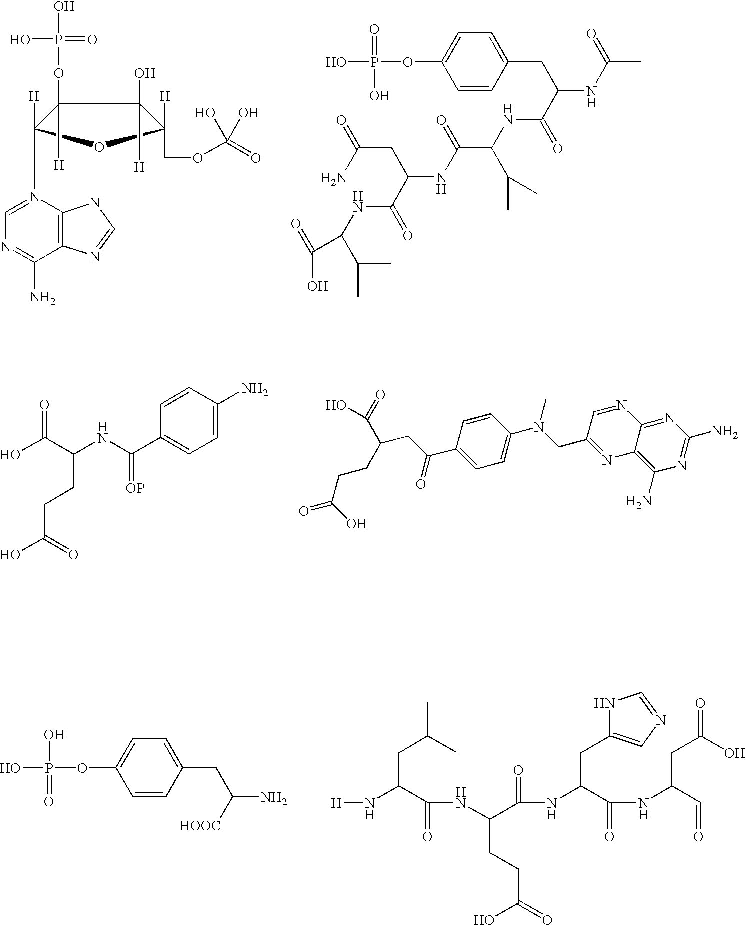

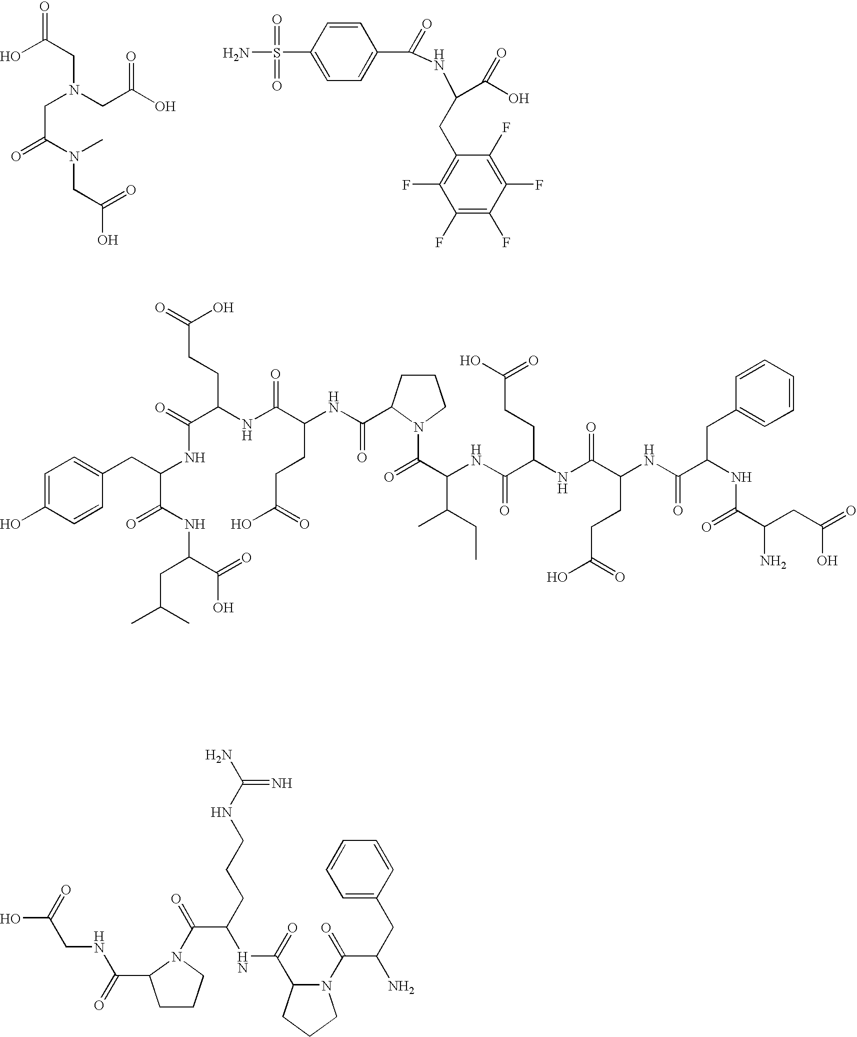

1. A method for the production of a ligand-anchor conjugate, comprising:
a) immobilisation or synthesis of an anchor molecule on a solid phase that is suitable for chemical synthesis;
b) synthesis of a ligand on an anchor molecule or binding of a ligand to the anchor molecule to form a ligand-anchor conjugate; and
c) cleavage of the formed ligand-anchor conjugate from the solid phase,
wherein the anchor molecule comprises at least one structural unit that is capable of immobilizing the ligand-anchor conjugate on a sensor surface, as well as at least one structural unit that enables the formation of a self-assembled monolayer on the sensor surface, and wherein the anchor molecule is terminally functionalized for binding with a ligand or a non-ligand.

2. The method according to claim 1, wherein a multitude of different ligand-anchor conjugates is generated using combinatorial methods for ligand synthesis.

3. The method according to claim 1, wherein the solid phase used for synthesis is a synthesis resin, a synthesis polymer film or a silicon or silicate surface.

4. The method according to claim 1, wherein the solid phase is a synthesis resin selected from a hydroxy resin, an amino resin, a trityl resin, a dihydropyrane resin, a carboxy resin or an arylsiloxy resin.

5. The method according to claim 1, wherein the structural unit that enables the formation of a self-assembled monolayer is a branched or unbranched, optionally substituted, saturated or partially unsaturated hydrocarbon chain which may be interrupted by heteroatoms, aromatic or heterocyclic units and comprises 2-2000 atoms.

6. The method according to claim 1, wherein the structural unit that enables the formation of a self-assembled monolayer is a hydrophobic structural unit R¹ which is formed by a branched or unbranched hydrocarbon chain of 1 to 50 carbon atoms which may be saturated or partially unsaturated.

7. The method according to claim 1, wherein the structural unit that enables the formation of a self-assembled monolayer comprises a branched or unbranched hydrophilic spacer R² which is formed by a hydrocarbon chain, which is interrupted by heteroatoms and comprises 2 to 1000 carbon atoms.

8. The method according to claim 1, wherein the structural unit that is capable of immobilizing the ligand-anchor conjugate on a sensor surface is a disulfide, thiol or sulfide group.

9. The method according to claim 1, wherein the terminal functionalization of the anchor molecule for binding with a ligand is a hydroxyl, amino or carboxyl group.

10. The method according to claim 1, wherein the wherein the ligand is selected from the group consisting of a protein, peptide, oligonucleotide, carbohydrate, isoprenoide, enzyme, lipid structure, saccharide, antibody, peptide hormone, cytokine, antibiotic, or an organic molecule having a molecular weight ≧50 g/mol.

11. A biosensor, comprising a multitude of identical or different ligand-anchor-conjugates immobilized on a sensor surface to form a biospecific boundary layer, wherein the ligand-anchor-conjugates are made by the method of claim 1.

12. The biosensor according to claim 11, additionally comprising anchor molecules exclusively combined with non-ligands.

13. The biosensor according to claim 11, wherein the sensor surface is fully or partially formed by gold, silver, palladium or platinum.

14. The biosensor according to claim 11, wherein the sensor surface comprises an array of positionally addressable fields on which the ligand-anchor-conjugates are immobilized.

15. The biosensor according to claim 14, wherein the fields are localized in cavities on the sensor surface.

16. The biosensor according to claim 14, wherein the ligand-anchor conjugates immobilized on the fields form a molecular library in which the ligands used differ between the respective fields.

17. A method for providing a biospecific boundary layer on a sensor surface, comprising the production of ligand-anchor conjugates by the method of claim 1, and additionally comprising the step of contacting the obtained ligand-anchor conjugates with a sensor surface.

18. A method of claim 17, wherein a solution of the ligand is applied in a defined manner on spatially separate sections of the sensor surface.

19. A method for detecting an interaction between ligands and receptors, comprising the step of contacting the receptors with a biosensor of claim 11.

20. The method according to claim 19, wherein the biosensor interacts with one or more receptors selected from proteins, DNA, RNA, oligonucleotides, prosthetic groups, vitamins, lipids, mono-, oligo- or polysaccharides or fusion proteins or synthesized primers.
